(19) United States
(12) Patent Application Publication
(10) Pub. No.: US 2026/0078132 A1
(43) Pub. Date: Mar. 19, 2026

(54) SOLID FORMS OF AN INHIBITOR OF HIF AND METHODS OF USING SAME

(71) Applicant: ONCOSPHERIX INC., Atlanta, GA (US)

(72) Inventors: Daniel LEVY, San Mateo, CA (US); Margaret OFFERMANN, Atlanta, GA (US); Ronald MUELLER, Northbrook, IL (US)

(73) Assignee: ONCOSPHERIX INC., Atlanta, GA (US)

(21) Appl. No.: 19/110,083

(22) PCT Filed: Sep. 11, 2023
(86) PCT No.: PCT/US2023/073833

§ 371 Date: Mar. 9, 2025

Related U.S. Application Data
(60) Provisional application No. 63/375,270, filed on Sep. 12, 2022.

(51) Int. Cl. C07D 491/052

(52) U.S. Cl. CPC C07D 491/052

(57) ABSTRACT

The disclosure is directed to novel solid-state forms of Compound 9 and pharmaceutically acceptable salts of Compound 9, compositions comprising the same, and methods of using the same, including use in treating cancer, hypoxia related pathologies, disorders leading to ischemia, and non-cancerous angiogenic diseases.

This application claims the benefit under 35 U.S.C. § 119(e) to U.S. Provisional Application No. 63/375,270 filed Sep. 12, 2022, which application is incorporated by reference herein in its entirety.

This disclosure provides solid-state forms of a compound that inhibits the function of the hypoxia inducible factor 1 (HIF-1) transcription factor and methods of using those solid-state forms including to treat cancer, hypoxia related pathologies, disorders leading to ischemia, for example stroke and ischemic heart disease, and non-cancerous angiogenic diseases.

HIFs are heterodimers consisting of an O₂-sensitive alpha subunit (HIF-1α, 2α, or 3α) and the constitutively expressed HIF-1β subunit (also called ARNT) [Huang et al. 2003]. Under normoxic conditions, a subunits are hydroxylated by a family of prolylhydroxylases, ubiquitylated in a Von Hippel-Lindau (VHL) protein-dependent manner (an E3 ligase) and degraded in the proteasome [Kaelin et al. 2008]. Under hypoxic conditions, the prolylhydroxylases cannot bind HIF-α units, and HIF-α is no longer degraded. This allows formation of HIF heterodimers that translocate to the nucleus, bind to hypoxia-response elements (HREs) in the regulatory regions of many genes, recruit co-activators p300 or CBP (paralogs often referred to as p300/CBP) and the polymerase II transcription machinery to transcribe over 100 target genes [Wenger et al. 2005]. The alpha subunits are also stabilized when the VHL protein is mutated and unable to target them for degradation.

N-Cyclobutyl-N-((2,2-dimethyl-2H-pyrano[3,2-b]pyridin-6-yl)methyl)-3,4-dimethoxybenzenesulfonamide (referred to herein as “Compound 9”) is an inhibitor of the function of the HIF-1 transcription factor. The chemical structure of Compound 9 is shown below:

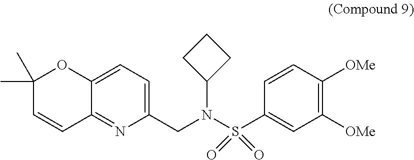 [SMILES: COc1ccc(S(=O)(=O)N(Cc2ccc3c(n2)C=CC(C)(C)O3)C2CCC2)cc1OC]

Without wishing to be bound by theory, Compound 9 is not believed to inhibit HIF-1a levels or HIF-1α-HIF-1β heterodimer formation during hypoxia, but rather binds to the CH1 domain of the transcription co-activator paralogs p300/CBP, thereby preventing the assembly of critical co-activator proteins on the nascent HIF transcription complex on the hypoxia response elements during hypoxia [Dong et al. 2019; Razafinjatovo 2016; Yin et al. 2012; Ferguson et al. 2017-A; Kim et al. 2017](FIG. 1). Data supporting the described mechanism of action include co-TP experiments between HIF-1a and p300/CBP, bead affinity pull-down of p300 by Compound 9, and cellular thermal shift assays showing the binding of Compound 9 to p300 [Dong et al. 2019; Shi et al. 2012; Mooring et al. 2011; Tan et al. 2011; Yin et al. 2012; Ferguson et al. 2017-B; Mun et al. 2012].

Further disclosure regarding Compound 9, including its method of preparation and methods of use, may be found in U.S. Pat. No. 9,062,072; Dong et al. 2019; and Mooring et al., 2011; each of which are incorporated herein by reference in its entirety.

### SUMMARY OF THE DISCLOSURE

The present disclosure relates to a novel solid-state form of Compound 9 and novel solid-state forms of pharmaceutically acceptable salts of Compound 9.

Another aspect of the disclosure provides methods of treatment or prevention of a hypoxia-related pathology comprising administering to a subject in need thereof an effective amount of at least one entity chosen from solid-state forms of Compound 9 and pharmaceutically acceptable salts of Compound 9. In some embodiments, the subject is suffering from a cancer. In some embodiments, the cancer is a solid tumor cancer.

In some embodiments, the methods disclosed herein comprise administering to the subject a pharmaceutical composition comprising an effective amount of at least one entity chosen from solid-state forms of Compound 9 and pharmaceutically acceptable salts of Compound 9.

In some embodiments, the methods of treating include administration of at least one additional active agent to the subject in need thereof, either in the same pharmaceutical composition as the at least one entity, or as separate compositions.

### BRIEF DESCRIPTION OF THE DRAWINGS

FIG. 1 depicts a possible mechanism of action for inhibition of HIF-1 transcription factor with Compound 9 and compares said mechanism to drugs that have undergone some clinical testing and whose antitumor effects are reportedly primarily through blocking HIF.

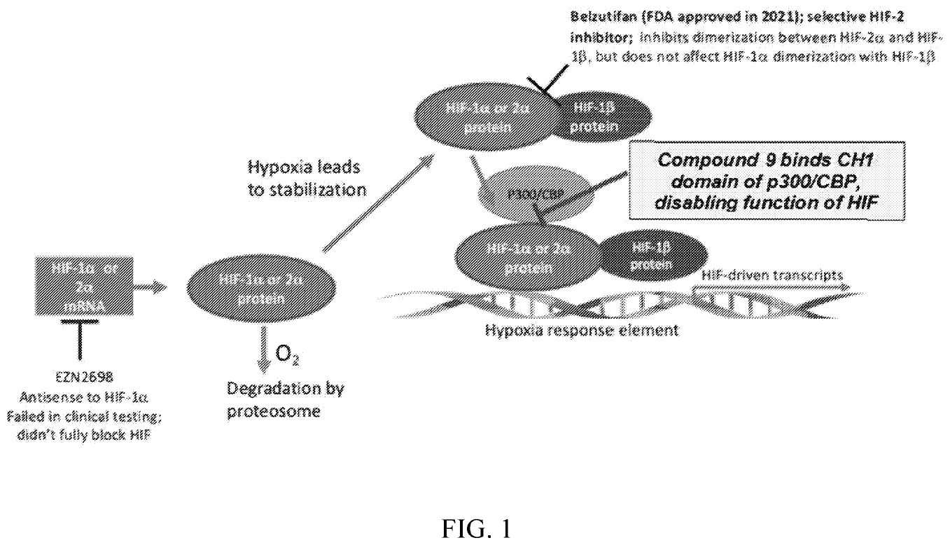

FIG. 2 depicts an XRPD diffractogram of Form A of Compound 9.

FIG. 3 depicts a TGA thermogram and DSC curve of Form A of Compound 9.

FIG. 4 depicts an XRPD diffractogram of Form A of Compound 9 mesylate salt.

FIG. 5 depicts a DSC curve of Form A of Compound 9 mesylate salt.

FIG. 6 depicts a TGA thermogram of Form A of Compound 9 mesylate salt.

FIG. 7 depicts an XRPD diffractogram of Form A of Compound 9 hydrochloride salt.

FIG. 8 depicts a TGA thermogram and DSC curve of Form A of Compound 9 hydrochloride salt.

FIG. 9 depicts an XRPD diffractogram of Form B of Compound 9 hydrochloride salt.

FIG. 10 depicts a TGA thermogram and DSC curve of Form B of Compound 9 hydrochloride salt.

FIG. 11 depicts an XRPD diffractogram of Form C of Compound 9 hydrochloride salt.

FIG. 12 depicts a TGA thermogram and DSC curve of Form C of Compound 9 hydrochloride salt.

FIG. 13 depicts an XRPD diffractogram of Form A of Compound 9 sulfate salt (pre-vacuum and pre-DVS treatments).

FIG. 14 depicts a TGA thermogram and DSC curve of Form A of Compound 9 sulfate salt.

FIG. 15 depicts a DVS curve of Form A of Compound 9 sulfate salt.

FIG. 16 depicts an XRPD diffractogram of Form B of Compound 9 sulfate salt (after performing DVS on Form A).

FIG. 17 depicts an XRPD diffractogram of Form A of Compound 9 tosylate salt.

FIG. 18 depicts a TGA thermogram and DSC curve of Form A of Compound 9 tosylate salt.

FIG. 19 depicts a DVS curve of Form A of Compound 9 tosylate salt.

FIG. 20 depicts an XRPD diffractogram of Form A of Compound 9 naphthalene-1,5-disulfonic acid hemi salt.

FIG. 21 depicts a TGA thermogram and DSC curve of Form A of Compound 9 naphthalene-1,5-disulfonic acid hemi salt.

FIG. 22 depicts a DVS curve of Form A of Compound 9 naphthalene-1,5-disulfonic acid hemi salt.

FIG. 23 illustrates the synthesis of Compound 9.

FIG. 24 depicts a ¹H NMR spectrum for Compound 9 using DMSO-D₆ as the solvent.

FIG. 25 depicts a ¹H NMR spectrum for Compound 9 using CDCl₃ as the solvent.

FIG. 26 depicts a ¹H NMR spectrum for Compound 9 mesylate salt using CDCl₃ as the solvent.

FIG. 27 depicts a ¹H NMR spectrum for Compound 9 hydrochloride salt using CDCl₃ as the solvent.

FIG. 28 depicts a ¹H NMR spectrum for Compound 9 sulfate salt using CDCl₃ as the solvent.

FIG. 29 depicts an XRPD diffractogram of Form A/B of Compound 9 sulfate salt (after placing Form A under vacuum).

FIG. 30 depicts a ¹H NMR spectrum for Compound 9 tosylate salt using CDCl₃ as the solvent.

FIG. 31 depicts a ¹H NMR spectrum for Compound 9 naphthalene-1,5-disulfonic acid hemi salt using CDCl₃ as the solvent.

### DETAILED DESCRIPTION OF THE DISCLOSURE

Disclosed herein are novel solid-state forms of Compound 9 and pharmaceutically acceptable salts of Compound 9.

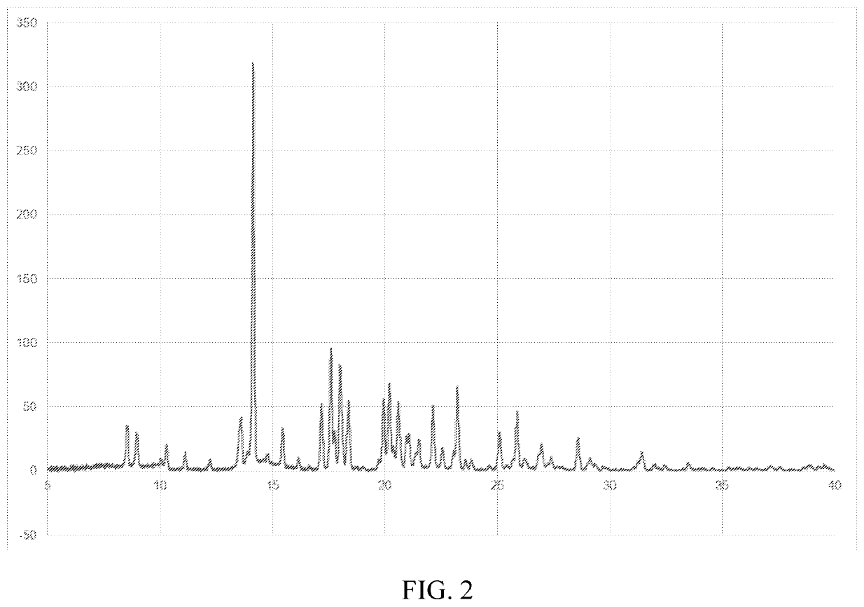

In some embodiments, at least one entity is Form A of Compound 9. In some embodiments, Form A of Compound 9 is characterized by an X-ray powder diffractogram substantially similar to that in FIG. 2. In some embodiments, Form A of Compound 9 is characterized by an X-ray powder diffractogram having a signal at at least two two-theta values chosen from 14.1±0.2, 17.6±0.2, 18.0±0.2, 20.2±0.2, and 23.3±0.2. In some embodiments, Form A of Compound 9 is characterized by an X-ray powder diffractogram having a signal at at least three two-theta values chosen from 14.1±0.2, 17.6±0.2, 18.0±0.2, 20.2±0.2, and 23.3±0.2. In some embodiments, Form A of Compound 9 is characterized by an X-ray powder diffractogram having a signal at at least four two-theta values chosen from 14.1±0.2, 17.6±0.2, 18.0±0.2, 20.2±0.2, and 23.3±0.2. In some embodiments, Form A of Compound 9 is characterized by an X-ray powder diffractogram having a signal at the following two-theta values 14.1±0.2, 17.6±0.2, 18.0±0.2, 20.2±0.2, and 23.3±0.2.

In some embodiments, Form A of Compound 9 is characterized by an X-ray powder diffractogram having a signal at at least two two-theta values chosen from 14.1±0.2, 17.2±0.2, 17.6±0.2, 18.0±0.2, 18.4±0.2, 20.0±0.2, 20.2±0.2, 20.6±0.2, 22.2±0.2, 23.3±0.2, and 25.9±0.2. In some embodiments, Form A of Compound 9 is characterized by an X-ray powder diffractogram having a signal at at least three two-theta values chosen from 14.1±0.2, 17.2±0.2, 17.6±0.2, 18.0±0.2, 18.4±0.2, 20.0±0.2, 20.2±0.2, 20.6±0.2, 22.2±0.2, 23.3±0.2, and 25.9±0.2. In some embodiments, Form A of Compound 9 is characterized by an X-ray powder diffractogram having a signal at at least four two-theta values chosen from 14.1±0.2, 17.2±0.2, 17.6±0.2, 18.0±0.2, 18.4±0.2, 20.0±0.2, 20.2±0.2, 20.6±0.2, 22.2±0.2, 23.3±0.2, and 25.9±0.2. In some embodiments, Form A of Compound 9 is characterized by an X-ray powder diffractogram having a signal at at least five two-theta values chosen from 14.1±0.2, 17.2±0.2, 17.6±0.2, 18.0±0.2, 18.4±0.2, 20.0±0.2, 20.2±0.2, 20.6±0.2, 22.2±0.2, 23.3±0.2, and 25.9±0.2. In some embodiments, Form A of Compound 9 is characterized by an X-ray powder diffractogram having a signal at at least six two-theta values chosen from 14.1±0.2, 17.2±0.2, 17.6±0.2, 18.0±0.2, 18.4±0.2, 20.0±0.2, 20.2±0.2, 20.6±0.2, 22.2±0.2, 23.3±0.2, and 25.9±0.2. In some embodiments, Form A of Compound 9 is characterized by an X-ray powder diffractogram having a signal at the following two-theta values 14.1±0.2, 17.2±0.2, 17.6±0.2, 18.0±0.2, 18.4±0.2, 20.0±0.2, 20.2±0.2, 20.6±0.2, 22.2±0.2, 23.3±0.2, and 25.9±0.2.

In some embodiments, Compound 9 is a crystalline solid consisting of 1% to 99% Form A relative to the total weight of the crystalline solid Compound 9. In some embodiments, the crystalline solid consists of 2% to 99% Form A relative to the total weight of the crystalline solid Compound 9. In some embodiments, the crystalline solid consists of 5% to 99% Form A relative to the total weight of the crystalline solid Compound 9. In some embodiments, the crystalline solid consists of 10% to 99% Form A relative to the total weight of the crystalline solid Compound 9. In some embodiments, the crystalline solid consists of 15% to 99% Form A relative to the total weight of the crystalline solid Compound 9. In some embodiments, the crystalline solid consists of 20% to 99% Form A relative to the total weight of the crystalline solid Compound 9. In some embodiments, the crystalline solid consists of 25% to 99% Form A relative to the total weight of the crystalline solid Compound 9. In some embodiments, the crystalline solid consists of 30% to 99% Form A relative to the total weight of the crystalline solid Compound 9. In some embodiments, the crystalline solid consists of 35% to 99% Form A relative to the total weight of the crystalline solid Compound 9. In some embodiments, the crystalline solid consists of 45% to 99% Form A relative to the total weight of the crystalline solid Compound 9. In some embodiments, the crystalline solid consists of 50% to 99% Form A relative to the total weight of the crystalline solid Compound 9. In some embodiments, the crystalline solid consists of 55% to 99% Form A relative to the total weight of the crystalline solid Compound 9. In some embodiments, the crystalline solid consists of 60% to 99% Form A relative to the total weight of the crystalline solid Compound 9. In some embodiments, the crystalline solid consists of 65% to 99% Form A relative to the total weight of the crystalline solid Compound 9. In some embodiments, the crystalline solid consists of 70% to 99% Form A relative to the total weight of the crystalline solid Compound 9. In some embodiments, the crystalline solid consists of 75% to 99% Form A relative to the total weight of the crystalline solid Compound 9. In some embodiments, the crystalline solid consists of 80% to 99% Form A relative to the total weight of the crystalline solid Compound 9. In some embodiments, the crystalline solid consists of 85% to 99% Form A relative to the total weight of the crystalline solid Compound 9. In some embodiments, the crystalline solid consists of 90% to 99% Form A relative to the total weight of the crystalline solid Compound 9. In some embodiments, the crystalline solid consists of 95% to 99% Form A relative to the total weight of the crystalline solid Compound 9.

In some embodiments, disclosed herein is a composition comprising Form A of Compound 9. In some embodiments, disclosed herein is a composition comprising Compound 9 in substantially pure Form A. In some embodiments, disclosed herein is a composition comprising at least one active compound consisting essentially of Compound 9 in Form A.

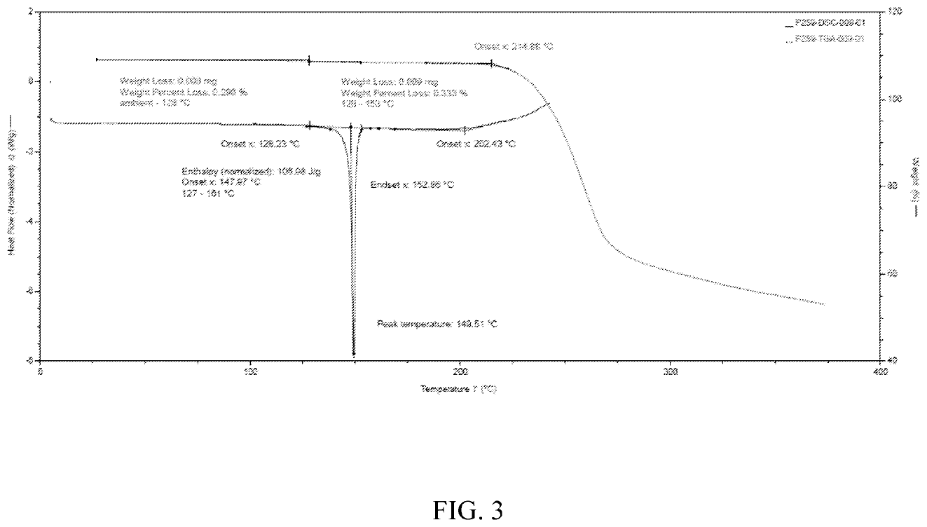

In some embodiments, Form A of Compound 9 is characterized by a DSC substantially similar to that in FIG. 3. In some embodiments, Form A of Compound 9 is characterized by a DSC having a melting onset at about 148° C. with a peak at about 150° C. In some embodiments, Form A of Compound 9 is characterized by a DSC having a peak in a range of 147° C. to 151° C.

In some embodiments, Form A of Compound 9 is characterized by a TGA substantially similar to that in FIG. 3. In some embodiments, Form A of Compound 9 is characterized by a TGA showing a weight loss of about 0.6% w/w from ambient temperature up to 153° C.

In some embodiments, Compound 9 is a crystalline solid consisting of 1% to 99% Form A relative to the total weight of the crystalline solid Compound 9. In some embodiments, the crystalline solid consists of 2% to 99% Form A relative to the total weight of the crystalline solid Compound 9. In some embodiments, the crystalline solid consists of 5% to 99% Form A relative to the total weight of the crystalline solid Compound 9. In some embodiments, the crystalline solid consists of 10% to 99% Form A relative to the total weight of the crystalline solid Compound 9. In some embodiments, the crystalline solid consists of 15% to 99% Form A relative to the total weight of the crystalline solid Compound 9. In some embodiments, the crystalline solid consists of 20% to 99% Form A relative to the total weight of the crystalline solid Compound 9. In some embodiments, the crystalline solid consists of 25% to 99% Form A relative to the total weight of the crystalline solid Compound 9. In some embodiments, the crystalline solid consists of 30% to 99% Form A relative to the total weight of the crystalline solid Compound 9. In some embodiments, the crystalline solid consists of 35% to 99% Form A relative to the total weight of the crystalline solid Compound 9. In some embodiments, the crystalline solid consists of 45% to 99% Form A relative to the total weight of the crystalline solid Compound 9. In some embodiments, the crystalline solid consists of 50% to 99% Form A relative to the total weight of the crystalline solid Compound 9. In some embodiments, the crystalline solid consists of 55% to 99% Form A relative to the total weight of the crystalline solid Compound 9. In some embodiments, the crystalline solid consists of 60% to 99% Form A relative to the total weight of the crystalline solid Compound 9. In some embodiments, the crystalline solid consists of 65% to 99% Form A relative to the total weight of the crystalline solid Compound 9. In some embodiments, the crystalline solid consists of 70% to 99% Form A relative to the total weight of the crystalline solid Compound 9. In some embodiments, the crystalline solid consists of 75% to 99% Form A relative to the total weight of the crystalline solid Compound 9. In some embodiments, the crystalline solid consists of 80% to 99% Form A relative to the total weight of the crystalline solid Compound 9. In some embodiments, the crystalline solid consists of 85% to 99% Form A relative to the total weight of the crystalline solid Compound 9. In some embodiments, the crystalline solid consists of 90% to 99% Form A relative to the total weight of the crystalline solid Compound 9. In some embodiments, the crystalline solid consists of 95% to 99% Form A relative to the total weight of the crystalline solid Compound 9.

In some embodiments, disclosed herein is a composition comprising Form A of Compound 9. In some embodiments, disclosed herein is a composition comprising Compound 9 in substantially pure Form A. In some embodiments, disclosed herein is a composition comprising at least one active compound consisting essentially of Compound 9 in Form A.

Another aspect of the disclosure provides pharmaceutical compositions comprising Form A of Compound 9. In some embodiments, the pharmaceutical composition comprising the at least one entity is administered to a subject in need thereof.

In some embodiments, at least one entity is chosen from solid-state forms of pharmaceutically acceptable salts of Compound 9. In some embodiments, the pharmaceutically acceptable salt is chosen from Form A of Compound 9 mesylate salt, Form A of Compound 9 hydrochloride salt, Form B of Compound 9 hydrochloride salt, Form C of Compound 9 hydrochloride salt, Form A of Compound 9 sulfate salt, Form B of Compound 9 sulfate salt, Form A of Compound 9 tosylate salt, and Form A of Compound 9 naphthalene-1,5-disulfonic acid hemi salt.

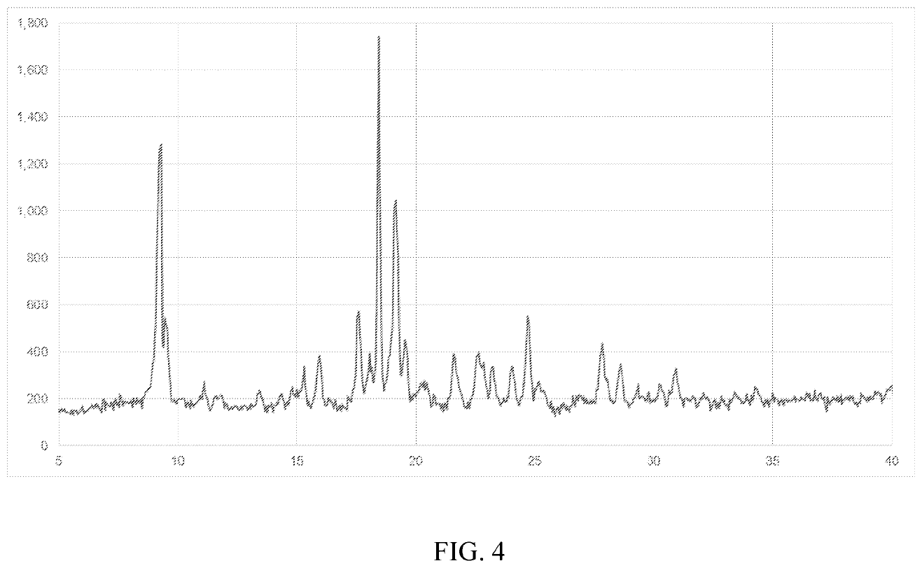

In some embodiments, Form A of Compound 9 mesylate salt is characterized by an X-ray powder diffractogram substantially similar to that in FIG. 4. In some embodiments, Form A of Compound 9 mesylate salt is characterized by an X-ray powder diffractogram having a signal at at least two two-theta values chosen from 9.3±0.2, 18.5±0.2, and 19.2±0.2. In some embodiments, Form A of Compound 9 mesylate salt is characterized by an X-ray powder diffractogram having a signal at the following two-theta values 9.3±0.2, 18.5±0.2, and 19.2±0.2.

In some embodiments, Form A of Compound 9 mesylate salt is characterized by an X-ray powder diffractogram having a signal at at least two two-theta values chosen from 9.3±0.2, 17.6±0.2, 18.5±0.2, 19.2±0.2, and 24.7±0.2. In some embodiments, Form A of Compound 9 mesylate salt is characterized by an X-ray powder diffractogram having a signal at at least three two-theta values chosen from 9.3±0.2, 17.6±0.2, 18.5±0.2, 19.2±0.2, and 24.7±0.2. In some embodiments, Form A of Compound 9 mesylate salt is characterized by an X-ray powder diffractogram having a signal at at least four two-theta values chosen from 9.3±0.2, 17.6±0.2, 18.5±0.2, 19.2±0.2, and 24.7±0.2. In some embodiments, Form A of Compound 9 mesylate salt is characterized by an X-ray powder diffractogram having a signal at the following two-theta values 9.3±0.2, 17.6±0.2, 18.5±0.2, 19.2±0.2, and 24.7±0.2.

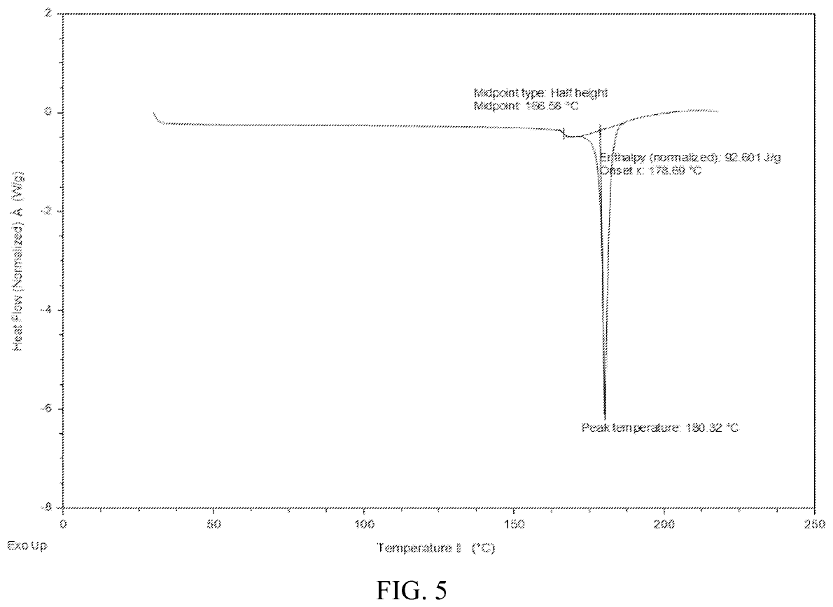

In some embodiments, Form A of Compound 9 mesylate salt is characterized by a DSC substantially similar to that in FIG. 5. In some embodiments, Form A of Compound 9 mesylate salt is characterized by a DSC having a melting onset at about 179° C. with a peak at about 180° C. In some embodiments, Form A of Compound 9 mesylate salt is characterized by a DSC having a peak in a range of 178° C. to 181° C.

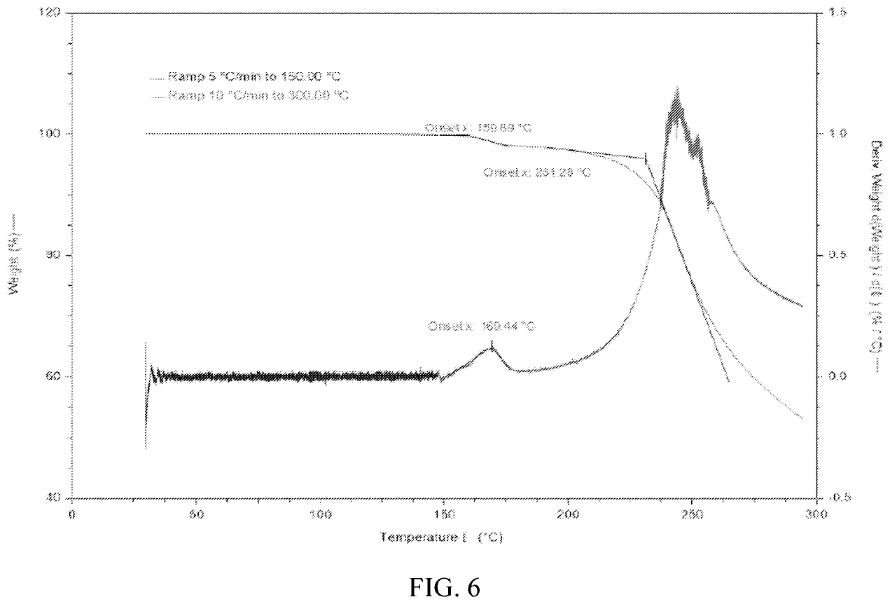

In some embodiments, Form A of Compound 9 mesylate salt is characterized by a TGA substantially similar to that in FIG. 6. In some embodiments, Form A of Compound 9 mesylate salt is characterized by a TGA showing a weight loss of about 2% w/w from ambient temperature up to 200° C.

In some embodiments, the pharmaceutically acceptable salt of Compound 9 is a crystalline solid consisting of 1% to 99% Form A of Compound 9 mesylate salt relative to the total weight of the crystalline solid Compound 9 mesylate salt. In some embodiments, the crystalline solid consists of 2% to 99% Form A of Compound 9 mesylate salt relative to the total weight of the crystalline solid Compound 9 mesylate salt. In some embodiments, the crystalline solid consists of 5% to 99% Form A of Compound 9 mesylate salt relative to the total weight of the crystalline solid Compound 9 mesylate salt. In some embodiments, the crystalline solid consists of 10% to 99% Form A of Compound 9 mesylate salt relative to the total weight of the crystalline solid Compound 9 mesylate salt. In some embodiments, the crystalline solid consists of 15% to 99% Form A of Compound 9 mesylate salt relative to the total weight of the crystalline solid Compound 9 mesylate salt. In some embodiments, the crystalline solid consists of 20% to 99% Form A of Compound 9 mesylate salt relative to the total weight of the crystalline solid Compound 9 mesylate salt. In some embodiments, the crystalline solid consists of 25% to 99% Form A of Compound 9 mesylate salt relative to the total weight of the crystalline solid Compound 9 mesylate salt. In some embodiments, the crystalline solid consists of 30% to 99% Form A of Compound 9 mesylate salt relative to the total weight of the crystalline solid Compound 9 mesylate salt. In some embodiments, the crystalline solid consists of 35% to 99% Form A of Compound 9 mesylate salt relative to the total weight of the crystalline solid Compound 9 mesylate salt. In some embodiments, the crystalline solid consists of 45% to 99% Form A of Compound 9 mesylate salt relative to the total weight of the crystalline solid Compound 9 mesylate salt. In some embodiments, the crystalline solid consists of 50% to 99% Form A of Compound 9 mesylate salt relative to the total weight of the crystalline solid Compound 9 mesylate salt. In some embodiments, the crystalline solid consists of 55% to 99% Form A of Compound 9 mesylate salt relative to the total weight of the crystalline solid Compound 9 mesylate salt. In some embodiments, the crystalline solid consists of 60% to 99% Form A of Compound 9 mesylate salt relative to the total weight of the crystalline solid Compound 9 mesylate salt. In some embodiments, the crystalline solid consists of 65% to 99% Form A of Compound 9 mesylate salt relative to the total weight of the crystalline solid Compound 9 mesylate salt. In some embodiments, the crystalline solid consists of 70% to 99% Form A of Compound 9 mesylate salt relative to the total weight of the crystalline solid Compound 9 mesylate salt. In some embodiments, the crystalline solid consists of 75% to 99% Form A of Compound 9 mesylate salt relative to the total weight of the crystalline solid Compound 9 mesylate salt. In some embodiments, the crystalline solid consists of 80% to 99% Form A of Compound 9 mesylate salt relative to the total weight of the crystalline solid Compound 9 mesylate salt. In some embodiments, the crystalline solid consists of 85% to 99% Form A of Compound 9 mesylate salt relative to the total weight of the crystalline solid Compound 9 mesylate salt. In some embodiments, the crystalline solid consists of 90% to 99% Form A of Compound 9 mesylate salt relative to the total weight of the crystalline solid Compound 9 mesylate salt. In some embodiments, the crystalline solid consists of 95% to 99% Form A of Compound 9 mesylate salt relative to the total weight of the crystalline solid Compound 9 mesylate salt.

In some embodiments, disclosed herein is a composition comprising Form A of Compound 9 mesylate salt. In some embodiments, disclosed herein is a composition comprising Compound 9 mesylate salt in substantially pure Form A. In some embodiments, disclosed herein is a composition comprising at least one active compound consisting essentially of Compound 9 mesylate salt in Form A.

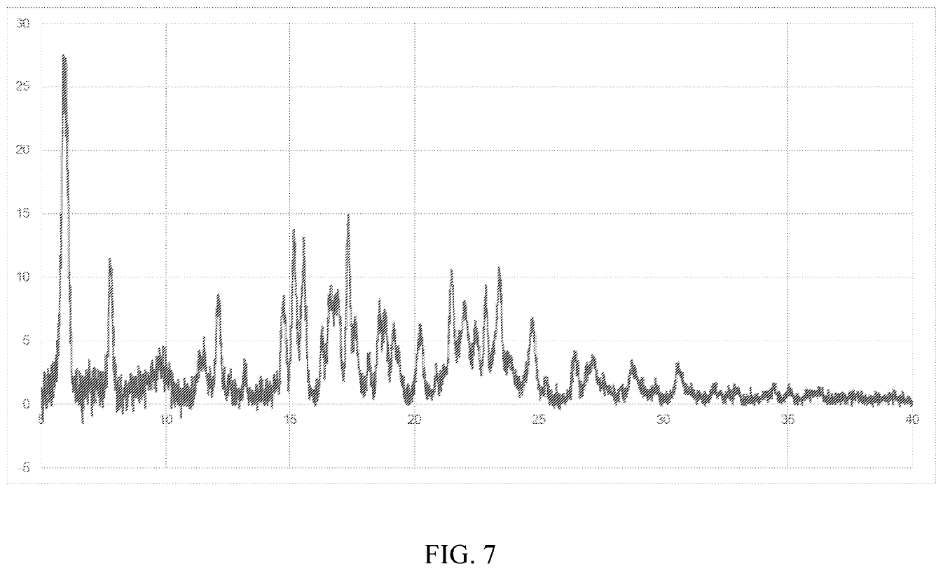

In some embodiments, Form A of Compound 9 hydrochloride salt is characterized by an X-ray powder diffractogram substantially similar to that in FIG. 7. In some embodiments, Form A of Compound 9 hydrochloride salt is characterized by an X-ray powder diffractogram having a signal at at least two two-theta values chosen from 6.0±0.2, 15.1±0.2, 15.5±0.2, and 17.3±0.2. In some embodiments, Form A of Compound 9 hydrochloride salt is characterized by an X-ray powder diffractogram having a signal at at least three two-theta values chosen from 6.0±0.2, 15.1±0.2, 15.5±0.2, and 17.3±0.2. In some embodiments, Form A of Compound 9 hydrochloride salt is characterized by an X-ray powder diffractogram having a signal at the following two-theta values 6.0±0.2, 15.1±0.2, 15.5±0.2, and 17.3±0.2.

In some embodiments, Form A of Compound 9 hydrochloride salt is characterized by an X-ray powder diffractogram having a signal at at least two two-theta values chosen from 6.0±0.2, 7.8±0.2, 15.1±0.2, 15.5±0.2, 17.3±0.2, and 21.5±0.2. In some embodiments, Form A of Compound 9 hydrochloride salt is characterized by an X-ray powder diffractogram having a signal at at least three two-theta values chosen from 6.0±0.2, 7.8±0.2, 15.1±0.2, 15.5±0.2, 17.3±0.2, and 21.5±0.2. In some embodiments, Form A of Compound 9 hydrochloride salt is characterized by an X-ray powder diffractogram having a signal at at least four two-theta values chosen from 6.0±0.2, 7.8±0.2, 15.1±0.2, 15.5±0.2, 17.3±0.2, and 21.5±0.2. In some embodiments, Form A of Compound 9 hydrochloride salt is characterized by an X-ray powder diffractogram having a signal at at least five two-theta values chosen from 6.0±0.2, 7.8±0.2, 15.1±0.2, 15.5±0.2, 17.3±0.2, and 21.5±0.2. In some embodiments, Form A of Compound 9 hydrochloride salt is characterized by an X-ray powder diffractogram having a signal at the following two-theta values 6.0±0.2, 7.8±0.2, 15.1±0.2, 15.5±0.2, 17.3±0.2, and 21.5±0.2.

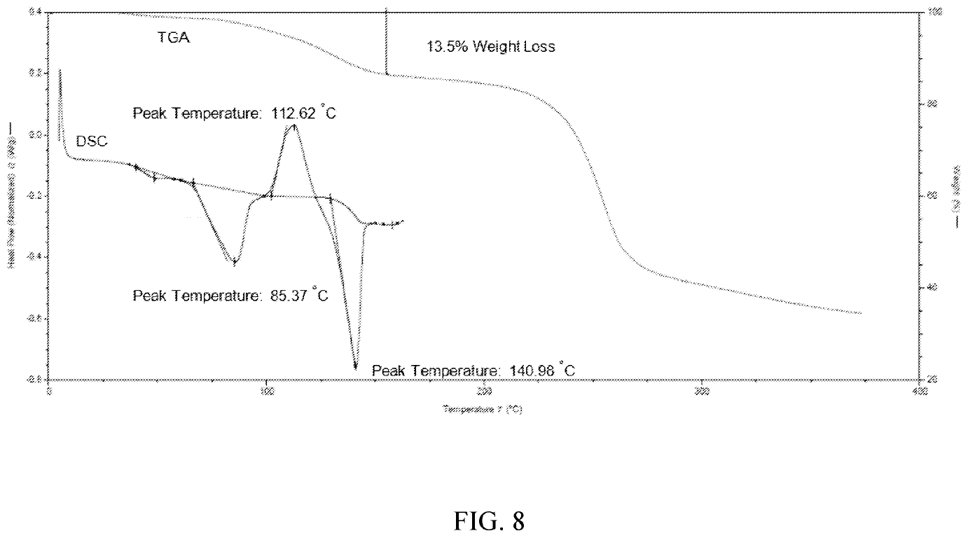

In some embodiments, Form A of Compound 9 hydrochloride salt is characterized by a DSC substantially similar to that in FIG. 8. In some embodiments, Form A of Compound 9 hydrochloride salt is characterized by a DSC having a transitionary melting onset at about 66° C. with a peak at about 85° C. In some embodiments, Form A of Compound 9 hydrochloride salt is characterized by a DSC having a terminal melting onset at about 130° C. with a peak at about 141° C. In some embodiments, Form A of Compound 9 hydrochloride salt is characterized by a DSC having a peak in a range of 129° C. to 142° C.

In some embodiments, Form A of Compound 9 hydrochloride salt is characterized by a TGA substantially similar to that in FIG. 8. In some embodiments, Form A of Compound 9 hydrochloride salt is characterized by a TGA showing a weight loss of about 13.5% w/w from ambient temperature up to 155° C.

In some embodiments, the pharmaceutically acceptable salt of Compound 9 is a crystalline solid consisting of 1% to 99% Form A of Compound 9 hydrochloride salt relative to the total weight of the crystalline solid Compound 9 hydrochloride salt. In some embodiments, the crystalline solid consists of 2% to 99% Form A of Compound 9 hydrochloride salt relative to the total weight of the crystalline solid Compound 9 hydrochloride salt. In some embodiments, the crystalline solid consists of 5% to 99% Form A of Compound 9 hydrochloride salt relative to the total weight of the crystalline solid Compound 9 hydrochloride salt. In some embodiments, the crystalline solid consists of 10% to 99% Form A of Compound 9 hydrochloride salt relative to the total weight of the crystalline solid Compound 9 hydrochloride salt. In some embodiments, the crystalline solid consists of 15% to 99% Form A of Compound 9 hydrochloride salt relative to the total weight of the crystalline solid Compound 9 hydrochloride salt. In some embodiments, the crystalline solid consists of 20% to 99% Form A of Compound 9 hydrochloride salt relative to the total weight of the crystalline solid Compound 9 hydrochloride salt. In some embodiments, the crystalline solid consists of 25% to 99% Form A of Compound 9 hydrochloride salt relative to the total weight of the crystalline solid Compound 9 hydrochloride salt. In some embodiments, the crystalline solid consists of 30% to 99% Form A of Compound 9 hydrochloride salt relative to the total weight of the crystalline solid Compound 9 hydrochloride salt. In some embodiments, the crystalline solid consists of 35% to 99% Form A of Compound 9 hydrochloride salt relative to the total weight of the crystalline solid Compound 9 hydrochloride salt. In some embodiments, the crystalline solid consists of 45% to 99% Form A of Compound 9 hydrochloride salt relative to the total weight of the crystalline solid Compound 9 hydrochloride salt. In some embodiments, the crystalline solid consists of 50% to 99% Form A of Compound 9 hydrochloride salt relative to the total weight of the crystalline solid Compound 9 hydrochloride salt. In some embodiments, the crystalline solid consists of 55% to 99% Form A of Compound 9 hydrochloride salt relative to the total weight of the crystalline solid Compound 9 hydrochloride salt. In some embodiments, the crystalline solid consists of 60% to 99% Form A of Compound 9 hydrochloride salt relative to the total weight of the crystalline solid Compound 9 hydrochloride salt. In some embodiments, the crystalline solid consists of 65% to 99% Form A of Compound 9 hydrochloride salt relative to the total weight of the crystalline solid Compound 9 hydrochloride salt. In some embodiments, the crystalline solid consists of 70% to 99% Form A of Compound 9 hydrochloride salt relative to the total weight of the crystalline solid Compound 9 hydrochloride salt. In some embodiments, the crystalline solid consists of 75% to 99% Form A of Compound 9 hydrochloride salt relative to the total weight of the crystalline solid Compound 9 hydrochloride salt. In some embodiments, the crystalline solid consists of 80% to 99% Form A of Compound 9 hydrochloride salt relative to the total weight of the crystalline solid Compound 9 hydrochloride salt. In some embodiments, the crystalline solid consists of 85% to 99% Form A of Compound 9 hydrochloride salt relative to the total weight of the crystalline solid Compound 9 hydrochloride salt. In some embodiments, the crystalline solid consists of 90% to 99% Form A of Compound 9 hydrochloride salt relative to the total weight of the crystalline solid Compound 9 hydrochloride salt. In some embodiments, the crystalline solid consists of 95% to 99% Form A of Compound 9 hydrochloride salt relative to the total weight of the crystalline solid Compound 9 hydrochloride salt.

In some embodiments, disclosed herein is a composition comprising Form A of Compound 9 hydrochloride salt. In some embodiments, disclosed herein is a composition comprising Compound 9 hydrochloride salt in substantially pure Form A. In some embodiments, disclosed herein is a composition comprising at least one active compound consisting essentially of Compound 9 hydrochloride salt in Form A.

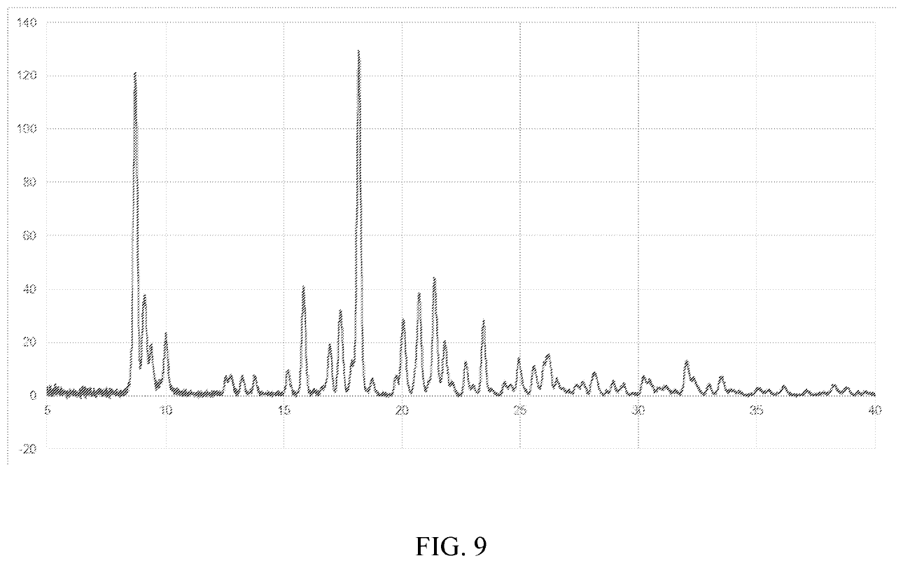

In some embodiments, Form B of Compound 9 hydrochloride salt is characterized by an X-ray powder diffractogram substantially similar to that in FIG. 9. In some embodiments, Form B of Compound 9 hydrochloride salt is characterized by an X-ray powder diffractogram having a signal at at least two two-theta values chosen from 8.7±0.2, 15.8±0.2, 18.2±0.2, and 21.3±0.2. In some embodiments, Form B of Compound 9 hydrochloride salt is characterized by an X-ray powder diffractogram having a signal at at least three two-theta values chosen from 8.7±0.2, 15.8±0.2, 18.2±0.2, and 21.3±0.2. In some embodiments, Form B of Compound 9 hydrochloride salt is characterized by an X-ray powder diffractogram having a signal at the following two-theta values 8.7±0.2, 15.8±0.2, 18.2±0.2, and 21.3±0.2.

In some embodiments, Form B of Compound 9 hydrochloride salt is characterized by an X-ray powder diffractogram having a signal at at least two two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2. In some embodiments, Form B of Compound 9 hydrochloride salt is characterized by an X-ray powder diffractogram having a signal at at least three two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2. In some embodiments, Form B of Compound 9 hydrochloride salt is characterized by an X-ray powder diffractogram having a signal at at least four two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2. In some embodiments, Form B of Compound 9 hydrochloride salt is characterized by an X-ray powder diffractogram having a signal at at least five two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2. In some embodiments, Form B of Compound 9 hydrochloride salt is characterized by an X-ray powder diffractogram having a signal at at least six two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2. In some embodiments, Form B of Compound 9 hydrochloride salt is characterized by an X-ray powder diffractogram having a signal at the following two-theta values 8.7±0.2, 9.1±0.2, 10.0±0.2, 15.8±0.2, 17.5±0.2, 18.2±0.2, 20.1±0.2, 20.7±0.2, and 21.3±0.2.

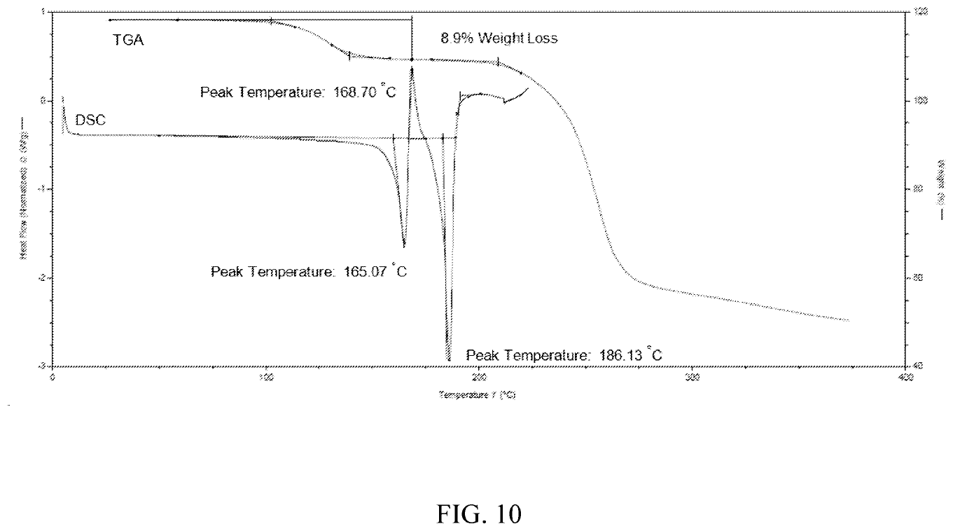

In some embodiments, Form B of Compound 9 hydrochloride salt is characterized by a DSC substantially similar to that in FIG. 10. In some embodiments, Form B of Compound 9 hydrochloride salt is characterized by a DSC having a transitionary melting onset at about 160° C. with a peak at about 165° C. In some embodiments, Form B of Compound 9 hydrochloride salt is characterized by a DSC having a terminal melting onset at about 183° C. with a peak at about 186° C. In some embodiments, Form B of Compound 9 hydrochloride salt is characterized by a DSC having a peak in a range of 184° C. to 187° C.

In some embodiments, Form B of Compound 9 hydrochloride salt is characterized by a TGA substantially similar to that in FIG. 10. In some embodiments, Form B of Compound 9 hydrochloride salt is characterized by a TGA showing a weight loss of about 8.9% w/w from ambient temperature up to 169° C.

In some embodiments, the pharmaceutically acceptable salt of Compound 9 is a crystalline solid consisting of 1% to 99% Form B of Compound 9 hydrochloride salt relative to the total weight of the crystalline solid Compound 9 hydrochloride salt. In some embodiments, the crystalline solid consists of 2% to 99% Form B of Compound 9 hydrochloride salt relative to the total weight of the crystalline solid Compound 9 hydrochloride salt. In some embodiments, the crystalline solid consists of 5% to 99% Form B of Compound 9 hydrochloride salt relative to the total weight of the crystalline solid Compound 9 hydrochloride salt. In some embodiments, the crystalline solid consists of 10% to 99% Form B of Compound 9 hydrochloride salt relative to the total weight of the crystalline solid Compound 9 hydrochloride salt. In some embodiments, the crystalline solid consists of 15% to 99% Form B of Compound 9 hydrochloride salt relative to the total weight of the crystalline solid Compound 9 hydrochloride salt. In some embodiments, the crystalline solid consists of 20% to 99% Form B of Compound 9 hydrochloride salt relative to the total weight of the crystalline solid Compound 9 hydrochloride salt. In some embodiments, the crystalline solid consists of 25% to 99% Form B of Compound 9 hydrochloride salt relative to the total weight of the crystalline solid Compound 9 hydrochloride salt. In some embodiments, the crystalline solid consists of 30% to 99% Form B of Compound 9 hydrochloride salt relative to the total weight of the crystalline solid Compound 9 hydrochloride salt. In some embodiments, the crystalline solid consists of 35% to 99% Form B of Compound 9 hydrochloride salt relative to the total weight of the crystalline solid Compound 9 hydrochloride salt. In some embodiments, the crystalline solid consists of 45% to 99% Form B of Compound 9 hydrochloride salt relative to the total weight of the crystalline solid Compound 9 hydrochloride salt. In some embodiments, the crystalline solid consists of 50% to 99% Form B of Compound 9 hydrochloride salt relative to the total weight of the crystalline solid Compound 9 hydrochloride salt. In some embodiments, the crystalline solid consists of 55% to 99% Form B of Compound 9 hydrochloride salt relative to the total weight of the crystalline solid Compound 9 hydrochloride salt. In some embodiments, the crystalline solid consists of 60% to 99% Form B of Compound 9 hydrochloride salt relative to the total weight of the crystalline solid Compound 9 hydrochloride salt. In some embodiments, the crystalline solid consists of 65% to 99% Form B of Compound 9 hydrochloride salt relative to the total weight of the crystalline solid Compound 9 hydrochloride salt. In some embodiments, the crystalline solid consists of 70% to 99% Form B of Compound 9 hydrochloride salt relative to the total weight of the crystalline solid Compound 9 hydrochloride salt. In some embodiments, the crystalline solid consists of 75% to 99% Form B of Compound 9 hydrochloride salt relative to the total weight of the crystalline solid Compound 9 hydrochloride salt. In some embodiments, the crystalline solid consists of 80% to 99% Form B of Compound 9 hydrochloride salt relative to the total weight of the crystalline solid Compound 9 hydrochloride salt. In some embodiments, the crystalline solid consists of 85% to 99% Form B of Compound 9 hydrochloride salt relative to the total weight of the crystalline solid Compound 9 hydrochloride salt. In some embodiments, the crystalline solid consists of 90% to 99% Form B of Compound 9 hydrochloride salt relative to the total weight of the crystalline solid Compound 9 hydrochloride salt. In some embodiments, the crystalline solid consists of 95% to 99% Form B of Compound 9 hydrochloride salt relative to the total weight of the crystalline solid Compound 9 hydrochloride salt.

In some embodiments, disclosed herein is a composition comprising Form B of Compound 9 hydrochloride salt. In some embodiments, disclosed herein is a composition comprising Compound 9 hydrochloride salt in substantially pure Form B. In some embodiments, disclosed herein is a composition comprising at least one active compound consisting essentially of Compound 9 hydrochloride salt in Form B.

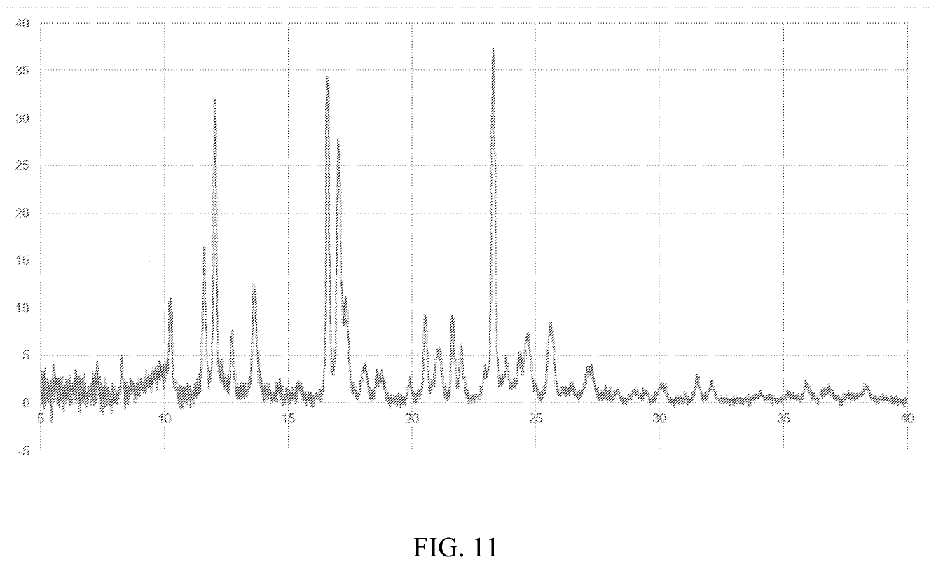

In some embodiments, Form C of Compound 9 hydrochloride salt is characterized by an X-ray powder diffractogram substantially similar to that in FIG. 11. In some embodiments, Form C of Compound 9 hydrochloride salt is characterized by an X-ray powder diffractogram having a signal at at least two two-theta values chosen from 11.6±0.2, 12.0±0.2, 16.6±0.2, 17.1±0.2, and 23.3±0.2. In some embodiments, Form C of Compound 9 hydrochloride salt is characterized by an X-ray powder diffractogram having a signal at at least three two-theta values chosen from 11.6±0.2, 12.0±0.2, 16.6±0.2, 17.1±0.2, and 23.3±0.2. In some embodiments, Form C of Compound 9 hydrochloride salt is characterized by an X-ray powder diffractogram having a signal at at least four two-theta values chosen from 11.6±0.2, 12.0±0.2, 16.6±0.2, 17.1±0.2, and 23.3±0.2. In some embodiments, Form C of Compound 9 hydrochloride salt is characterized by an X-ray powder diffractogram having a signal at the following two-theta values 11.6±0.2, 12.0±0.2, 16.6±0.2, 17.1±0.2, and 23.3±0.2.

In some embodiments, Form C of Compound 9 hydrochloride salt is characterized by an X-ray powder diffractogram having a signal at at least two two-theta values chosen from 10.2±0.2, 11.6±0.2, 12.0±0.2, 13.7±0.2, 16.6±0.2, 17.1±0.2, and 23.3±0.2. In some embodiments, Form C of Compound 9 hydrochloride salt is characterized by an X-ray powder diffractogram having a signal at at least three two-theta values chosen from 10.2±0.2, 11.6±0.2, 12.0±0.2, 13.7±0.2, 16.6±0.2, 17.1±0.2, and 23.3±0.2. In some embodiments, Form C of Compound 9 hydrochloride salt is characterized by an X-ray powder diffractogram having a signal at at least four two-theta values chosen from 10.2±0.2, 11.6±0.2, 12.0±0.2, 13.7±0.2, 16.6±0.2, 17.1±0.2, and 23.3±0.2. In some embodiments, Form C of Compound 9 hydrochloride salt is characterized by an X-ray powder diffractogram having a signal at at least five two-theta values chosen from 10.2±0.2, 11.6±0.2, 12.0±0.2, 13.7±0.2, 16.6±0.2, 17.1±0.2, and 23.3±0.2. In some embodiments, Form C of Compound 9 hydrochloride salt is characterized by an X-ray powder diffractogram having a signal at at least six two-theta values chosen from 10.2±0.2, 11.6±0.2, 12.0±0.2, 13.7±0.2, 16.6±0.2, 17.1±0.2, and 23.3±0.2. In some embodiments, Form C of Compound 9 hydrochloride salt is characterized by an X-ray powder diffractogram having a signal at the following two-theta values 10.2±0.2, 11.6±0.2, 12.0±0.2, 13.7±0.2, 16.6±0.2, 17.1±0.2, and 23.3±0.2.

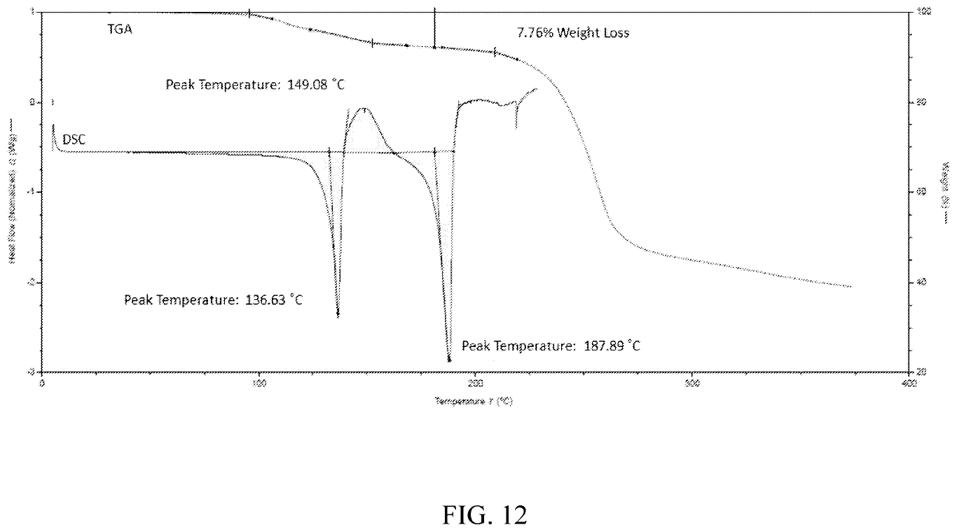

In some embodiments, Form C of Compound 9 hydrochloride salt is characterized by a DSC substantially similar to that in FIG. 12. In some embodiments, Form C of Compound 9 hydrochloride salt is characterized by a DSC having a transitionary melting onset at about 132° C. with a peak at about 137° C. In some embodiments, Form C of Compound 9 hydrochloride salt is characterized by a DSC having a terminal melting onset at about 181° C. with a peak at about 188° C. In some embodiments, Form C of Compound 9 hydrochloride salt is characterized by a DSC having a peak in a range of 180° C. to 189° C.

In some embodiments, Form C of Compound 9 hydrochloride salt is characterized by a TGA substantially similar to that in FIG. 12. In some embodiments, Form C of Compound 9 hydrochloride salt is characterized by a TGA showing a weight loss of about 7.8% w/w from ambient temperature up to 181° C.

In some embodiments, the pharmaceutically acceptable salt of Compound 9 is a crystalline solid consisting of 1% to 99% Form C of Compound 9 hydrochloride salt relative to the total weight of the crystalline solid Compound 9 hydrochloride salt. In some embodiments, the crystalline solid consists of 2% to 99% Form C of Compound 9 hydrochloride salt relative to the total weight of the crystalline solid Compound 9 hydrochloride salt. In some embodiments, the crystalline solid consists of 5% to 99% Form C of Compound 9 hydrochloride salt relative to the total weight of the crystalline solid Compound 9 hydrochloride salt. In some embodiments, the crystalline solid consists of 10% to 99% Form C of Compound 9 hydrochloride salt relative to the total weight of the crystalline solid Compound 9 hydrochloride salt. In some embodiments, the crystalline solid consists of 15% to 99% Form C of Compound 9 hydrochloride salt relative to the total weight of the crystalline solid Compound 9 hydrochloride salt. In some embodiments, the crystalline solid consists of 20% to 99% Form C of Compound 9 hydrochloride salt relative to the total weight of the crystalline solid Compound 9 hydrochloride salt. In some embodiments, the crystalline solid consists of 25% to 99% Form C of Compound 9 hydrochloride salt relative to the total weight of the crystalline solid Compound 9 hydrochloride salt. In some embodiments, the crystalline solid consists of 30% to 99% Form C of Compound 9 hydrochloride salt relative to the total weight of the crystalline solid Compound 9 hydrochloride salt. In some embodiments, the crystalline solid consists of 35% to 99% Form C of Compound 9 hydrochloride salt relative to the total weight of the crystalline solid Compound 9 hydrochloride salt. In some embodiments, the crystalline solid consists of 45% to 99% Form C of Compound 9 hydrochloride salt relative to the total weight of the crystalline solid Compound 9 hydrochloride salt. In some embodiments, the crystalline solid consists of 50% to 99% Form C of Compound 9 hydrochloride salt relative to the total weight of the crystalline solid Compound 9 hydrochloride salt. In some embodiments, the crystalline solid consists of 55% to 99% Form C of Compound 9 hydrochloride salt relative to the total weight of the crystalline solid Compound 9 hydrochloride salt. In some embodiments, the crystalline solid consists of 60% to 99% Form C of Compound 9 hydrochloride salt relative to the total weight of the crystalline solid Compound 9 hydrochloride salt. In some embodiments, the crystalline solid consists of 65% to 99% Form C of Compound 9 hydrochloride salt relative to the total weight of the crystalline solid Compound 9 hydrochloride salt. In some embodiments, the crystalline solid consists of 70% to 99% Form C of Compound 9 hydrochloride salt relative to the total weight of the crystalline solid Compound 9 hydrochloride salt. In some embodiments, the crystalline solid consists of 75% to 99% Form C of Compound 9 hydrochloride salt relative to the total weight of the crystalline solid Compound 9 hydrochloride salt. In some embodiments, the crystalline solid consists of 80% to 99% Form C of Compound 9 hydrochloride salt relative to the total weight of the crystalline solid Compound 9 hydrochloride salt. In some embodiments, the crystalline solid consists of 85% to 99% Form C of Compound 9 hydrochloride salt relative to the total weight of the crystalline solid Compound 9 hydrochloride salt. In some embodiments, the crystalline solid consists of 90% to 99% Form C of Compound 9 hydrochloride salt relative to the total weight of the crystalline solid Compound 9 hydrochloride salt. In some embodiments, the crystalline solid consists of 95% to 99% Form C of Compound 9 hydrochloride salt relative to the total weight of the crystalline solid Compound 9 hydrochloride salt.

In some embodiments, disclosed herein is a composition comprising Form C of Compound 9 hydrochloride salt. In some embodiments, disclosed herein is a composition comprising Compound 9 hydrochloride salt in substantially pure Form C. In some embodiments, disclosed herein is a composition comprising at least one active compound consisting essentially of Compound 9 hydrochloride salt in Form C.

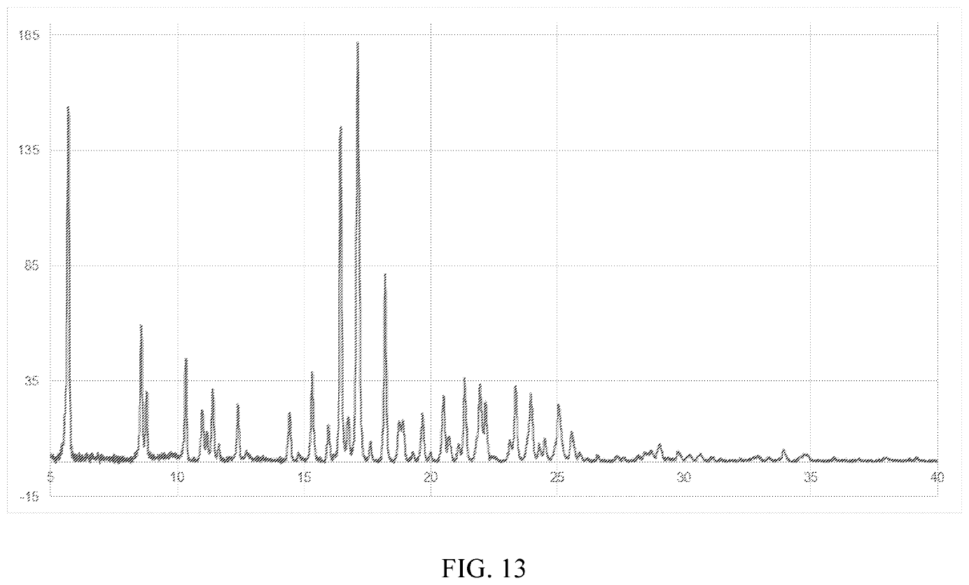

In some embodiments, Form A of Compound 9 sulfate salt is characterized by an X-ray powder diffractogram substantially similar to that in FIG. 13. In some embodiments, Form A of Compound 9 sulfate salt is characterized by an X-ray powder diffractogram having a signal at at least two two-theta values chosen from 5.7±0.2, 8.6±0.2, 16.4±0.2, 17.1±0.2, and 18.2±0.2. In some embodiments, Form A of Compound 9 sulfate salt is characterized by an X-ray powder diffractogram having a signal at at least three two-theta values chosen from 5.7±0.2, 8.6±0.2, 16.4±0.2, 17.1±0.2, and 18.2±0.2. In some embodiments, Form A of Compound 9 sulfate salt is characterized by an X-ray powder diffractogram having a signal at at least four two-theta values chosen from 5.7±0.2, 8.6±0.2, 16.4±0.2, 17.1±0.2, and 18.2±0.2. In some embodiments, Form A of Compound 9 sulfate salt is characterized by an X-ray powder diffractogram having a signal at the following two-theta values 5.7±0.2, 8.6±0.2, 16.4±0.2, 17.1±0.2, and 18.2±0.2.

In some embodiments, Form A of Compound 9 sulfate salt is characterized by an X-ray powder diffractogram having a signal at at least two two-theta values chosen from 5.7±0.2, 8.6±0.2, 8.8±0.2, 10.3±0.2, 15.3±0.2, 16.4±0.2, 17.1±0.2, and 18.2±0.2. In some embodiments, Form A of Compound 9 sulfate salt is characterized by an X-ray powder diffractogram having a signal at at least three two-theta values chosen from 5.7±0.2, 8.6±0.2, 8.8±0.2, 10.3±0.2, 15.3±0.2, 16.4±0.2, 17.1±0.2, and 18.2±0.2. In some embodiments, Form A of Compound 9 sulfate salt is characterized by an X-ray powder diffractogram having a signal at at least four two-theta values chosen from 5.7±0.2, 8.6±0.2, 8.8±0.2, 10.3±0.2, 15.3±0.2, 16.4±0.2, 17.1±0.2, and 18.2±0.2. In some embodiments, Form A of Compound 9 sulfate salt is characterized by an X-ray powder diffractogram having a signal at at least five two-theta values chosen from 5.7±0.2, 8.6±0.2, 8.8±0.2, 10.3±0.2, 15.3±0.2, 16.4±0.2, 17.1±0.2, and 18.2±0.2. In some embodiments, Form A of Compound 9 sulfate salt is characterized by an X-ray powder diffractogram having a signal at at least six two-theta values chosen from 5.7±0.2, 8.6±0.2, 8.8±0.2, 10.3±0.2, 15.3±0.2, 16.4±0.2, 17.1±0.2, and 18.2±0.2. In some embodiments, Form A of Compound 9 sulfate salt is characterized by an X-ray powder diffractogram having a signal at the following two-theta values 5.7±0.2, 8.6±0.2, 8.8±0.2, 10.3±0.2, 15.3±0.2, 16.4±0.2, 17.1±0.2, and 18.2±0.2.

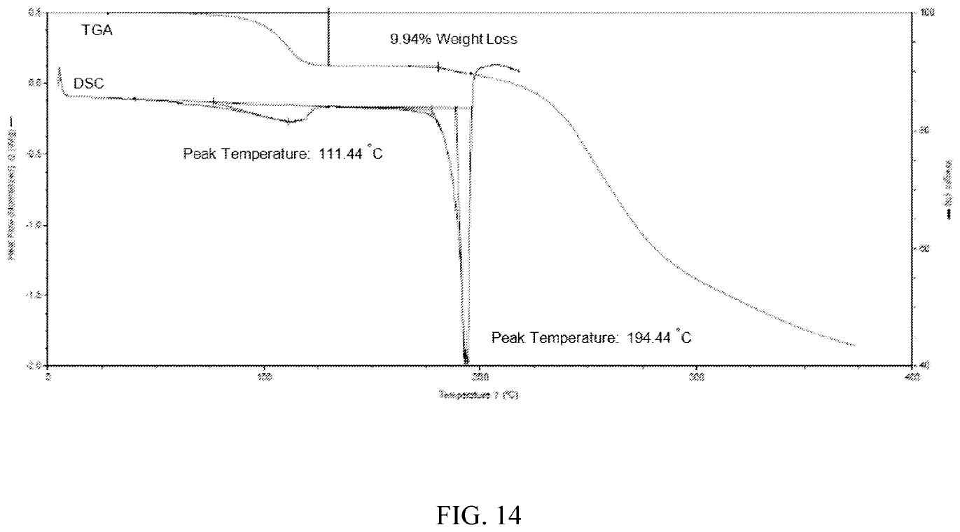

In some embodiments, Form A of Compound 9 sulfate salt is characterized by a DSC substantially similar to that in FIG. 14. In some embodiments, Form A of Compound 9 sulfate salt is characterized by a DSC having a terminal melting onset at about 177° C. with a peak at about 194° C. In some embodiments, Form A of Compound 9 sulfate salt is characterized by a DSC having a peak in a range of 170° C. to 195° C.

In some embodiments, Form A of Compound 9 sulfate salt is characterized by a TGA substantially similar to that in FIG. 14. In some embodiments, Form A of Compound 9 sulfate salt is characterized by a TGA showing a weight loss of about 9.9% w/w from ambient temperature up to 130° C.

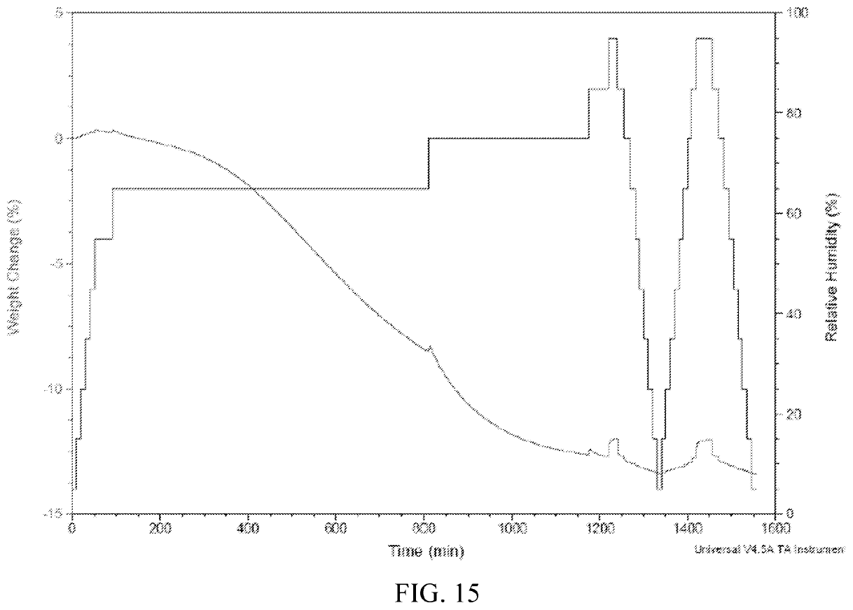

In some embodiments, Form A of Compound 9 sulfate salt is characterized by a DVS curve substantially similar to that in FIG. 15. In some embodiments, Form A of Compound 9 sulfate salt is characterized by a DVS showing adsorption of about 0.2% at 55% RH. In some embodiments, Form A of Compound 9 sulfate salt is characterized by a DVS showing desorption of about 12.9% at 85% RH. In some embodiments, Form A of Compound 9 sulfate salt is characterized by a DVS showing adsorption of about 0.7% at 95% RH.

In some embodiments, Form A of Compound 9 sulfate salt is characterized by a DVS showing desorption of from 10% to 15% at 95% RH. In some embodiments, Form A of Compound 9 sulfate salt is characterized by a DVS showing desorption of from 11% to 15% at 95% RH. In some embodiments, Form A of Compound 9 sulfate salt is characterized by a DVS showing desorption of from 12% to 15% at 95% RH. In some embodiments, Form A of Compound 9 sulfate salt is characterized by a DVS showing desorption of from 13% to 15% at 95% RH. In some embodiments, Form A of Compound 9 sulfate salt is characterized by a DVS showing desorption of from 12% to 14% at 95% RH. In some embodiments, Form A of Compound 9 sulfate salt is characterized by a DVS showing desorption of from 13% to 14% at 95% RH. In some embodiments, Form A of Compound 9 sulfate salt is characterized by a DVS showing desorption of from 13% to 15% at 95% RH.

In some embodiments, the pharmaceutically acceptable salt of Compound 9 is a crystalline solid consisting of 1% to 99% Form A of Compound 9 sulfate salt relative to the total weight of the crystalline solid Compound 9 sulfate salt. In some embodiments, the crystalline solid consists of 2% to 99% Form A of Compound 9 sulfate salt relative to the total weight of the crystalline solid Compound 9 sulfate salt. In some embodiments, the crystalline solid consists of 5% to 99% Form A of Compound 9 sulfate salt relative to the total weight of the crystalline solid Compound 9 sulfate salt. In some embodiments, the crystalline solid consists of 10% to 99% Form A of Compound 9 sulfate salt relative to the total weight of the crystalline solid Compound 9 sulfate salt. In some embodiments, the crystalline solid consists of 15% to 99% Form A of Compound 9 sulfate salt relative to the total weight of the crystalline solid Compound 9 sulfate salt. In some embodiments, the crystalline solid consists of 20% to 99% Form A of Compound 9 sulfate salt relative to the total weight of the crystalline solid Compound 9 sulfate salt. In some embodiments, the crystalline solid consists of 25% to 99% Form A of Compound 9 sulfate salt relative to the total weight of the crystalline solid Compound 9 sulfate salt. In some embodiments, the crystalline solid consists of 30% to 99% Form A of Compound 9 sulfate salt relative to the total weight of the crystalline solid Compound 9 sulfate salt. In some embodiments, the crystalline solid consists of 35% to 99% Form A of Compound 9 sulfate salt relative to the total weight of the crystalline solid Compound 9 sulfate salt. In some embodiments, the crystalline solid consists of 45% to 99% Form A of Compound 9 sulfate salt relative to the total weight of the crystalline solid Compound 9 sulfate salt. In some embodiments, the crystalline solid consists of 50% to 99% Form A of Compound 9 sulfate salt relative to the total weight of the crystalline solid Compound 9 sulfate salt. In some embodiments, the crystalline solid consists of 55% to 99% Form A of Compound 9 sulfate salt relative to the total weight of the crystalline solid Compound 9 sulfate salt. In some embodiments, the crystalline solid consists of 60% to 99% Form A of Compound 9 sulfate salt relative to the total weight of the crystalline solid Compound 9 sulfate salt. In some embodiments, the crystalline solid consists of 65% to 99% Form A of Compound 9 sulfate salt relative to the total weight of the crystalline solid Compound 9 sulfate salt. In some embodiments, the crystalline solid consists of 70% to 99% Form A of Compound 9 sulfate salt relative to the total weight of the crystalline solid Compound 9 sulfate salt. In some embodiments, the crystalline solid consists of 75% to 99% Form A of Compound 9 sulfate salt relative to the total weight of the crystalline solid Compound 9 sulfate salt. In some embodiments, the crystalline solid consists of 80% to 99% Form A of Compound 9 sulfate salt relative to the total weight of the crystalline solid Compound 9 sulfate salt. In some embodiments, the crystalline solid consists of 85% to 99% Form A of Compound 9 sulfate salt relative to the total weight of the crystalline solid Compound 9 sulfate salt. In some embodiments, the crystalline solid consists of 90% to 99% Form A of Compound 9 sulfate salt relative to the total weight of the crystalline solid Compound 9 sulfate salt. In some embodiments, the crystalline solid consists of 95% to 99% Form A of Compound 9 sulfate salt relative to the total weight of the crystalline solid Compound 9 sulfate salt.

In some embodiments, disclosed herein is a composition comprising Form A of Compound 9 sulfate salt. In some embodiments, disclosed herein is a composition comprising Compound 9 sulfate salt in substantially pure Form A. In some embodiments, disclosed herein is a composition comprising at least one active compound consisting essentially of Compound 9 sulfate salt in Form A.

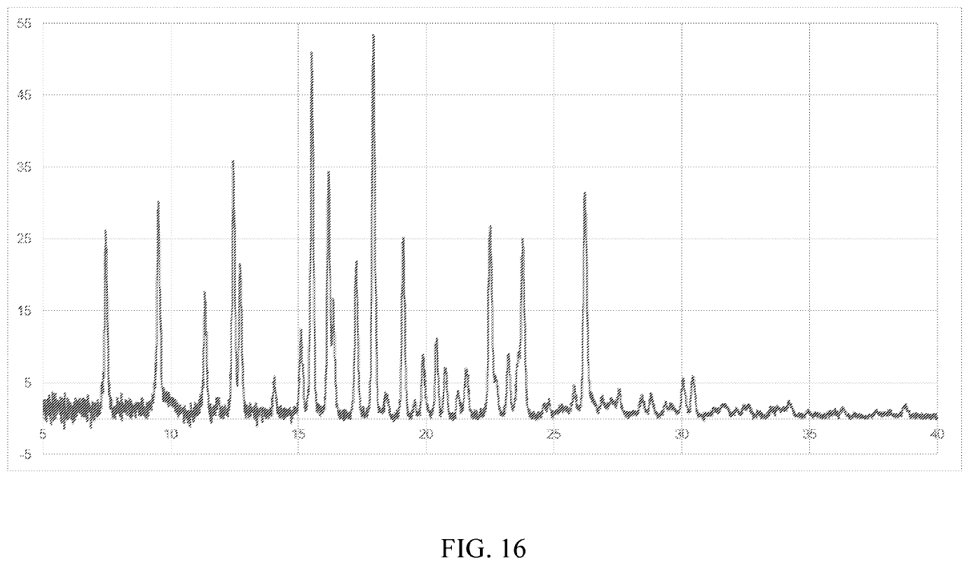

In some embodiments, Form B of Compound 9 sulfate salt is characterized by an X-ray powder diffractogram substantially similar to that in FIG. 16. In some embodiments, Form B of Compound 9 sulfate salt is characterized by an X-ray powder diffractogram having a signal at at least two two-theta values chosen from 9.5±0.2, 12.4±0.2, 15.5±0.2, 16.2±0.2, 18.0±0.2, and 26.2±0.2. In some embodiments, Form B of Compound 9 sulfate salt is characterized by an X-ray powder diffractogram having a signal at at least three two-theta values chosen from 9.5±0.2, 12.4±0.2, 15.5±0.2, 16.2±0.2, 18.0±0.2, and 26.2±0.2. In some embodiments, Form B of Compound 9 sulfate salt is characterized by an X-ray powder diffractogram having a signal at at least four two-theta values chosen from 9.5±0.2, 12.4±0.2, 15.5±0.2, 16.2±0.2, 18.0±0.2, and 26.2±0.2. In some embodiments, Form B of Compound 9 sulfate salt is characterized by an X-ray powder diffractogram having a signal at the following two-theta values 9.5±0.2, 12.4±0.2, 15.5±0.2, 16.2±0.2, 18.0±0.2, and 26.2±0.2.

In some embodiments, Form B of Compound 9 sulfate salt is characterized by an X-ray powder diffractogram having a signal at at least two two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2. In some embodiments, Form B of Compound 9 sulfate salt is characterized by an X-ray powder diffractogram having a signal at at least three two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2. In some embodiments, Form B of Compound 9 sulfate salt is characterized by an X-ray powder diffractogram having a signal at at least four two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2. In some embodiments, Form B of Compound 9 sulfate salt is characterized by an X-ray powder diffractogram having a signal at at least five two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2. In some embodiments, Form B of Compound 9 sulfate salt is characterized by an X-ray powder diffractogram having a signal at at least six two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2. In some embodiments, Form B of Compound 9 sulfate salt is characterized by an X-ray powder diffractogram having a signal at the following two-theta values 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2.

In some embodiments, the pharmaceutically acceptable salt of Compound 9 is a crystalline solid consisting of 1% to 99% Form B of Compound 9 sulfate salt relative to the total weight of the crystalline solid Compound 9 sulfate salt. In some embodiments, the crystalline solid consists of 2% to 99% Form B of Compound 9 sulfate salt relative to the total weight of the crystalline solid Compound 9 sulfate salt. In some embodiments, the crystalline solid consists of 5% to 99% Form B of Compound 9 sulfate salt relative to the total weight of the crystalline solid Compound 9 sulfate salt. In some embodiments, the crystalline solid consists of 10% to 99% Form B of Compound 9 sulfate salt relative to the total weight of the crystalline solid Compound 9 sulfate salt. In some embodiments, the crystalline solid consists of 15% to 99% Form B of Compound 9 sulfate salt relative to the total weight of the crystalline solid Compound 9 sulfate salt. In some embodiments, the crystalline solid consists of 20% to 99% Form B of Compound 9 sulfate salt relative to the total weight of the crystalline solid Compound 9 sulfate salt. In some embodiments, the crystalline solid consists of 25% to 99% Form B of Compound 9 sulfate salt relative to the total weight of the crystalline solid Compound 9 sulfate salt. In some embodiments, the crystalline solid consists of 30% to 99% Form B of Compound 9 sulfate salt relative to the total weight of the crystalline solid Compound 9 sulfate salt. In some embodiments, the crystalline solid consists of 35% to 99% Form B of Compound 9 sulfate salt relative to the total weight of the crystalline solid Compound 9 sulfate salt. In some embodiments, the crystalline solid consists of 45% to 99% Form B of Compound 9 sulfate salt relative to the total weight of the crystalline solid Compound 9 sulfate salt. In some embodiments, the crystalline solid consists of 50% to 99% Form B of Compound 9 sulfate salt relative to the total weight of the crystalline solid Compound 9 sulfate salt. In some embodiments, the crystalline solid consists of 55% to 99% Form B of Compound 9 sulfate salt relative to the total weight of the crystalline solid Compound 9 sulfate salt. In some embodiments, the crystalline solid consists of 60% to 99% Form B of Compound 9 sulfate salt relative to the total weight of the crystalline solid Compound 9 sulfate salt. In some embodiments, the crystalline solid consists of 65% to 99% Form B of Compound 9 sulfate salt relative to the total weight of the crystalline solid Compound 9 sulfate salt. In some embodiments, the crystalline solid consists of 70% to 99% Form B of Compound 9 sulfate salt relative to the total weight of the crystalline solid Compound 9 sulfate salt. In some embodiments, the crystalline solid consists of 75% to 99% Form B of Compound 9 sulfate salt relative to the total weight of the crystalline solid Compound 9 sulfate salt. In some embodiments, the crystalline solid consists of 80% to 99% Form B of Compound 9 sulfate salt relative to the total weight of the crystalline solid Compound 9 sulfate salt. In some embodiments, the crystalline solid consists of 85% to 99% Form B of Compound 9 sulfate salt relative to the total weight of the crystalline solid Compound 9 sulfate salt. In some embodiments, the crystalline solid consists of 90% to 99% Form B of Compound 9 sulfate salt relative to the total weight of the crystalline solid Compound 9 sulfate salt. In some embodiments, the crystalline solid consists of 95% to 99% Form B of Compound 9 sulfate salt relative to the total weight of the crystalline solid Compound 9 sulfate salt.

In some embodiments, disclosed herein is a composition comprising Form B of Compound 9 sulfate salt. In some embodiments, disclosed herein is a composition comprising Compound 9 sulfate salt in substantially pure Form B. In some embodiments, disclosed herein is a composition comprising at least one active compound consisting essentially of Compound 9 sulfate salt in Form B.

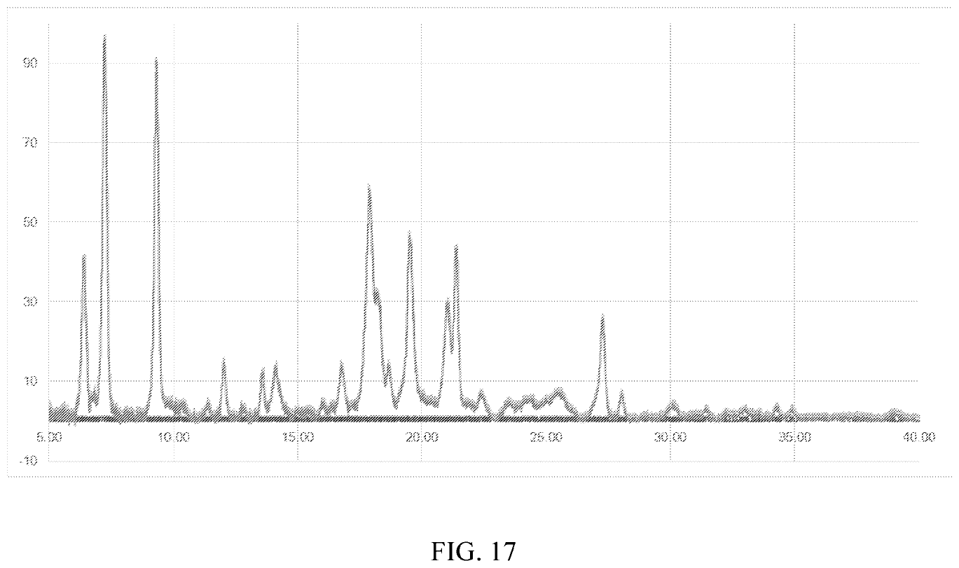

In some embodiments, Form A of Compound 9 tosylate salt is characterized by an X-ray powder diffractogram substantially similar to that in FIG. 17. In some embodiments, Form A of Compound 9 tosylate salt is characterized by an X-ray powder diffractogram having a signal at at least two two-theta values chosen from 7.3±0.2, 9.3±0.2, 17.9±0.2, and 19.5±0.2. In some embodiments, Form A of Compound 9 tosylate salt is characterized by an X-ray powder diffractogram having a signal at at least three two-theta values chosen from 7.3±0.2, 9.3±0.2, 17.9±0.2, and 19.5±0.2. In some embodiments, Form A of Compound 9 tosylate salt is characterized by an X-ray powder diffractogram having a signal at the following two-theta values 7.3±0.2, 9.3±0.2, 17.9±0.2, and 19.5±0.2.

In some embodiments, Form A of Compound 9 tosylate salt is characterized by an X-ray powder diffractogram having a signal at at least two two-theta values chosen from 6.4±0.2, 7.3±0.2, 9.3±0.2, 17.9±0.2, 19.5±0.2, 21.0±0.2, 21.4±0.2, and 27.3±0.2. In some embodiments, Form A of Compound 9 tosylate salt is characterized by an X-ray powder diffractogram having a signal at at least three two-theta values chosen from 6.4±0.2, 7.3±0.2, 9.3±0.2, 17.9±0.2, 19.5±0.2, 21.0±0.2, 21.4±0.2, and 27.3±0.2. In some embodiments, Form A of Compound 9 tosylate salt is characterized by an X-ray powder diffractogram having a signal at at least four two-theta values chosen from 6.4±0.2, 7.3±0.2, 9.3±0.2, 17.9±0.2, 19.5±0.2, 21.0±0.2, 21.4±0.2, and 27.3±0.2. In some embodiments, Form A of Compound 9 tosylate salt is characterized by an X-ray powder diffractogram having a signal at at least five two-theta values chosen from 6.4±0.2, 7.3±0.2, 9.3±0.2, 17.9±0.2, 19.5±0.2, 21.0±0.2, 21.4±0.2, and 27.3±0.2. In some embodiments, Form A of Compound 9 tosylate salt is characterized by an X-ray powder diffractogram having a signal at at least six two-theta values chosen from 6.4±0.2, 7.3±0.2, 9.3±0.2, 17.9±0.2, 19.5±0.2, 21.0±0.2, 21.4±0.2, and 27.3±0.2. In some embodiments, Form A of Compound 9 tosylate salt is characterized by an X-ray powder diffractogram having a signal at the following two-theta values 6.4±0.2, 7.3±0.2, 9.3±0.2, 17.9±0.2, 19.5±0.2, 21.0±0.2, 21.4±0.2, and 27.3±0.2.

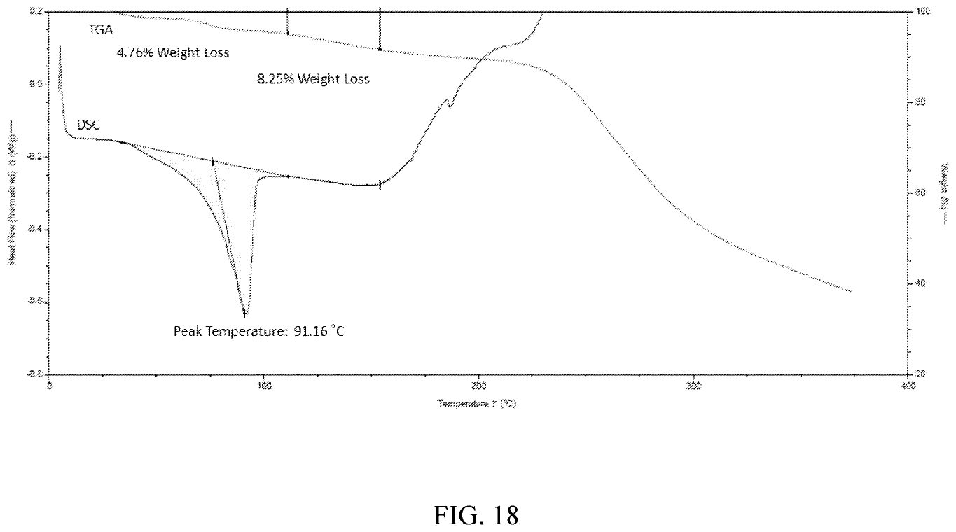

In some embodiments, Form A of Compound 9 tosylate salt is characterized by a DSC substantially similar to that in FIG. 18. In some embodiments, Form A of Compound 9 tosylate salt is characterized by a DSC having a melting onset at about 76° C. with a peak at about 91° C. In some embodiments, Form A of Compound 9 tosylate salt is characterized by a DSC having a peak in a range of 75° C. to 92° C.

In some embodiments, Form A of Compound 9 tosylate salt is characterized by a TGA substantially similar to that in FIG. 18. In some embodiments, Form A of Compound 9 tosylate salt is characterized by a TGA showing a weight loss of about 4.8% w/w from ambient temperature up to 111° C. In some embodiments, Form A of Compound 9 tosylate salt is characterized by a TGA showing a weight loss of about 8.3% w/w from ambient temperature up to 154° C.

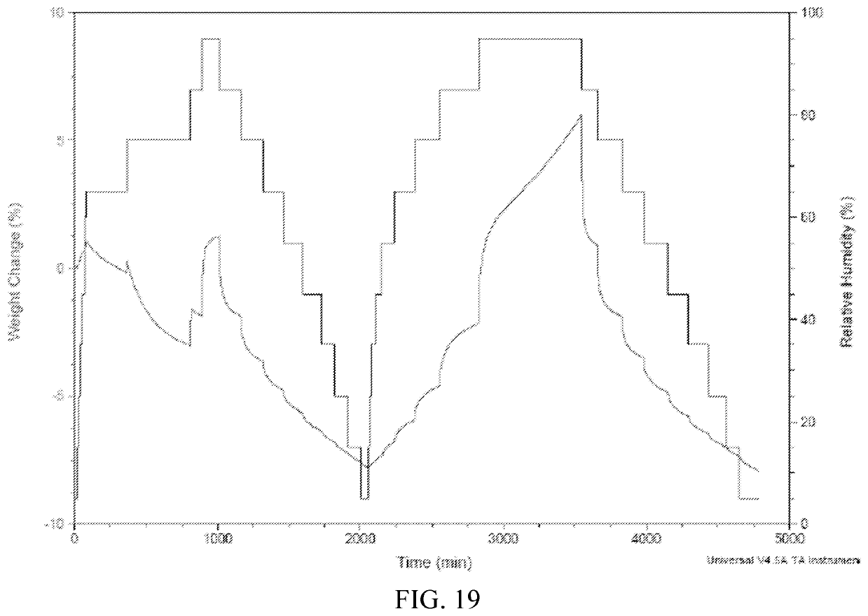

In some embodiments, Form A of Compound 9 tosylate salt is characterized by a DVS curve substantially similar to that in FIG. 19. In some embodiments, Form A of Compound 9 tosylate salt is characterized by a DVS showing adsorption of about 0.8% at 55% RH. In some embodiments, Form A of Compound 9 tosylate salt is characterized by a DVS showing desorption of about 3.9% as the RH was increased to 75%. In some embodiments, Form A of Compound 9 tosylate salt is characterized by a DVS showing adsorption of about 4.3% when the RH was increased from 75% to 95%. In some embodiments, Form A of Compound 9 tosylate salt is characterized by a DVS showing a total net mass increase of about 1.3% at 95% RH. In some embodiments, Form A of Compound 9 tosylate salt is characterized by a DVS showing a net mass decrease of about 9.0% when the RH was reduced from 95% to 5% in the first cycle.

In some embodiments, the pharmaceutically acceptable salt of Compound 9 is a crystalline solid consisting of 1% to 99% Form A of Compound 9 tosylate salt relative to the total weight of the crystalline solid Compound 9 tosylate salt. In some embodiments, the crystalline solid consists of 2% to 99% Form A of Compound 9 tosylate salt relative to the total weight of the crystalline solid Compound 9 tosylate salt. In some embodiments, the crystalline solid consists of 5% to 99% Form A of Compound 9 tosylate salt relative to the total weight of the crystalline solid Compound 9 tosylate salt. In some embodiments, the crystalline solid consists of 10% to 99% Form A of Compound 9 tosylate salt relative to the total weight of the crystalline solid Compound 9 tosylate salt. In some embodiments, the crystalline solid consists of 15% to 99% Form A of Compound 9 tosylate salt relative to the total weight of the crystalline solid Compound 9 tosylate salt. In some embodiments, the crystalline solid consists of 20% to 99% Form A of Compound 9 tosylate salt relative to the total weight of the crystalline solid Compound 9 tosylate salt. In some embodiments, the crystalline solid consists of 25% to 99% Form A of Compound 9 tosylate salt relative to the total weight of the crystalline solid Compound 9 tosylate salt. In some embodiments, the crystalline solid consists of 30% to 99% Form A of Compound 9 tosylate salt relative to the total weight of the crystalline solid Compound 9 tosylate salt. In some embodiments, the crystalline solid consists of 35% to 99% Form A of Compound 9 tosylate salt relative to the total weight of the crystalline solid Compound 9 tosylate salt. In some embodiments, the crystalline solid consists of 45% to 99% Form A of Compound 9 tosylate salt relative to the total weight of the crystalline solid Compound 9 tosylate salt. In some embodiments, the crystalline solid consists of 50% to 99% Form A of Compound 9 tosylate salt relative to the total weight of the crystalline solid Compound 9 tosylate salt. In some embodiments, the crystalline solid consists of 55% to 99% Form A of Compound 9 tosylate salt relative to the total weight of the crystalline solid Compound 9 tosylate salt. In some embodiments, the crystalline solid consists of 60% to 99% Form A of Compound 9 tosylate salt relative to the total weight of the crystalline solid Compound 9 tosylate salt. In some embodiments, the crystalline solid consists of 65% to 99% Form A of Compound 9 tosylate salt relative to the total weight of the crystalline solid Compound 9 tosylate salt. In some embodiments, the crystalline solid consists of 70% to 99% Form A of Compound 9 tosylate salt relative to the total weight of the crystalline solid Compound 9 tosylate salt. In some embodiments, the crystalline solid consists of 75% to 99% Form A of Compound 9 tosylate salt relative to the total weight of the crystalline solid Compound 9 tosylate salt. In some embodiments, the crystalline solid consists of 80% to 99% Form A of Compound 9 tosylate salt relative to the total weight of the crystalline solid Compound 9 tosylate salt. In some embodiments, the crystalline solid consists of 85% to 99% Form A of Compound 9 tosylate salt relative to the total weight of the crystalline solid Compound 9 tosylate salt. In some embodiments, the crystalline solid consists of 90% to 99% Form A of Compound 9 tosylate salt relative to the total weight of the crystalline solid Compound 9 tosylate salt. In some embodiments, the crystalline solid consists of 95% to 99% Form A of Compound 9 tosylate salt relative to the total weight of the crystalline solid Compound 9 tosylate salt.

In some embodiments, disclosed herein is a composition comprising Form A of Compound 9 tosylate salt. In some embodiments, disclosed herein is a composition comprising Compound 9 tosylate salt in substantially pure Form A. In some embodiments, disclosed herein is a composition comprising at least one active compound consisting essentially of Compound 9 tosylate salt in Form A.

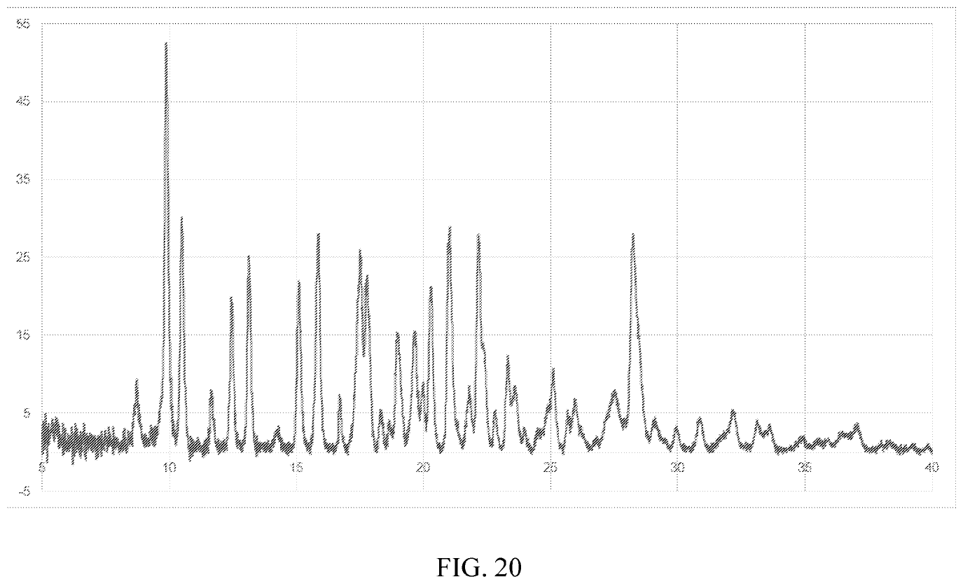

In some embodiments, Form A of Compound 9 naphthalene-1,5-disulfonic acid hemi salt is characterized by an X-ray powder diffractogram substantially similar to that in FIG. 20. In some embodiments, Form A of Compound 9 naphthalene-1,5-disulfonic acid hemi salt is characterized by an X-ray powder diffractogram having a signal at at least two two-theta values chosen from 9.9±0.2, 10.5±0.2, 15.8±0.2, 21.0±0.2, 22.2±0.2, and 28.2±0.2. In some embodiments, Form A of Compound 9 naphthalene-1,5-disulfonic acid hemi salt is characterized by an X-ray powder diffractogram having a signal at at least three two-theta values chosen from 9.9±0.2, 10.5±0.2, 15.8±0.2, 21.0±0.2, 22.2±0.2, and 28.2±0.2. In some embodiments, Form A of Compound 9 naphthalene-1,5-disulfonic acid hemi salt is characterized by an X-ray powder diffractogram having a signal at at least four two-theta values chosen from 9.9±0.2, 10.5±0.2, 15.8±0.2, 21.0±0.2, 22.2±0.2, and 28.2±0.2. In some embodiments, Form A of Compound 9 naphthalene-1,5-disulfonic acid hemi salt is characterized by an X-ray powder diffractogram having a signal at the following two-theta values 9.9±0.2, 10.5±0.2, 15.8±0.2, 21.0±0.2, 22.2±0.2, and 28.2±0.2.

In some embodiments, Form A of Compound 9 naphthalene-1,5-disulfonic acid hemi salt is characterized by an X-ray powder diffractogram having a signal at at least two two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2. In some embodiments, Form A of Compound 9 naphthalene-1,5-disulfonic acid hemi salt is characterized by an X-ray powder diffractogram having a signal at at least three two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2. In some embodiments, Form A of Compound 9 naphthalene-1,5-disulfonic acid hemi salt is characterized by an X-ray powder diffractogram having a signal at at least four two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2. In some embodiments, Form A of Compound 9 naphthalene-1,5-disulfonic acid hemi salt is characterized by an X-ray powder diffractogram having a signal at at least five two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2. In some embodiments, Form A of Compound 9 naphthalene-1,5-disulfonic acid hemi salt is characterized by an X-ray powder diffractogram having a signal at at least six two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2. In some embodiments, Form A of Compound 9 naphthalene-1,5-disulfonic acid hemi salt is characterized by an X-ray powder diffractogram having a signal at the following two-theta values 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2.

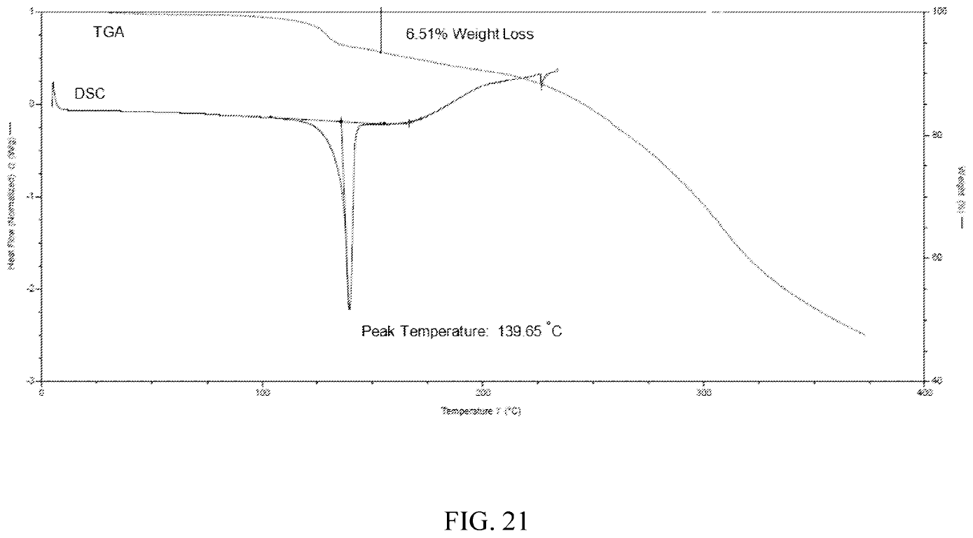

In some embodiments, Form A of Compound 9 naphthalene-1,5-disulfonic acid hemi salt is characterized by a DSC substantially similar to that in FIG. 21. In some embodiments, Form A of Compound 9 naphthalene-1,5-disulfonic acid hemi salt is characterized by a DSC having a melting onset at about 136° C. with a peak at about 140° C. In some embodiments, Form A of Compound 9 naphthalene-1,5-disulfonic acid hemi salt is characterized by a DSC having a peak in a range of 135° C. to 141° C.

In some embodiments, Form A of Compound 9 naphthalene-1,5-disulfonic acid hemi salt is characterized by a TGA substantially similar to that in FIG. 21. In some embodiments, Form A of Compound 9 naphthalene-1,5-disulfonic acid hemi salt is characterized by a TGA showing a weight loss of about 6.5% w/w from ambient temperature up to 154° C.

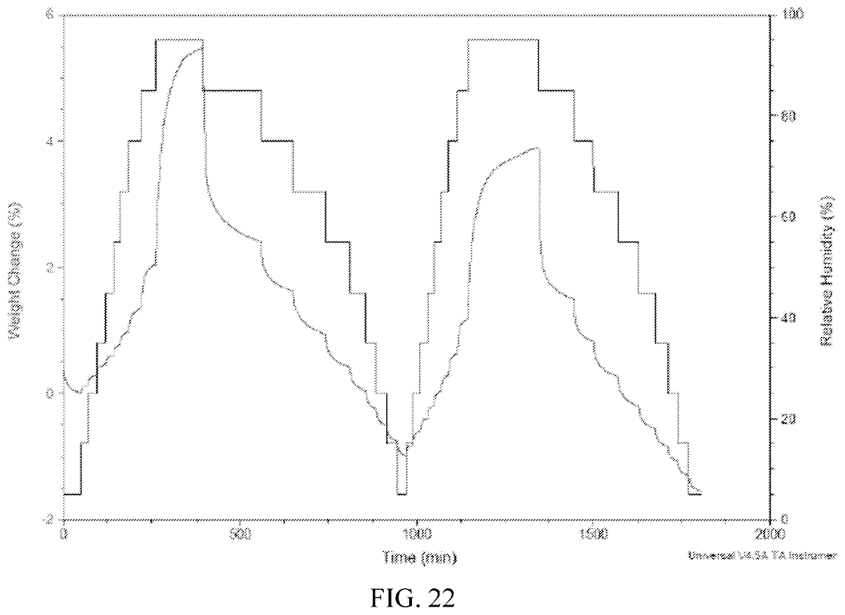

In some embodiments, Form A of Compound 9 naphthalene-1,5-disulfonic acid hemi salt is characterized by a DVS curve substantially similar to that in FIG. 22. In some embodiments, Form A of Compound 9 naphthalene-1,5-disulfonic acid hemi salt is characterized by a DVS showing adsorption of about 5.6% at 95% RH. In some embodiments, Form A of Compound 9 naphthalene-1,5-disulfonic acid hemi salt is characterized by a DVS showing adsorption of from 4% to 6% at 95% RH. In some embodiments, Form A of Compound 9 naphthalene-1,5-disulfonic acid hemi salt is characterized by a DVS showing adsorption of from 5% to 6% at 95% RH.

In some embodiments, the pharmaceutically acceptable salt of Compound 9 is a crystalline solid consisting of 1% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 2% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 5% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 10% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 15% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 20% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 25% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 30% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 35% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 45% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 50% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 55% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 60% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 65% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 70% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 75% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 80% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 85% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 90% to 99% Form A of Compound 9 naphthalene-1,5-disulfonic acid hemi salt relative to the total weight of the crystalline solid Compound 9 naphthalene-1,5-disulfonic acid hemi salt. In some embodiments, the crystalline solid consists of 95% to 99% Form A of Compound 9 naphthalene-1,5-disulfonic acid hemi salt relative to the total weight of the crystalline solid Compound 9 naphthalene-1,5-disulfonic acid hemi salt.

In some embodiments, disclosed herein is a composition comprising Form A of Compound 9 naphthalene-1,5-disulfonic acid hemi salt. In some embodiments, disclosed herein is a composition comprising Compound 9 naphthalene-1,5-disulfonic acid hemi salt in substantially pure Form A. In some embodiments, disclosed herein is a composition comprising at least one active compound consisting essentially of Compound 9 naphthalene-1,5-disulfonic acid hemi salt in Form A.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable carrier is chosen from pharmaceutically acceptable fillers, disintegrants, surfactants, binders, and lubricants.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

In some embodiments, the pharmaceutical composition is in a form suitable for oral administration to a patient. Example dosage forms include, but are not limited to, tablets, caplets, capsules (such as hard gelatin capsules and soft elastic gelatin capsules), cachets, troches, and lozenges.

The composition, shape, and type of dosage forms of the compositions of the disclosure will typically vary depending on their use. For example, a dosage form used in the acute treatment of a disease or disorder may contain larger amounts of the active ingredient, for example the disclosed compounds or combinations thereof, than a dosage form used in the chronic treatment of the same disease or disorder.

In some embodiments, methods of treatment or prophylaxis of a hypoxia-related pathology are provided comprising administering to a subject in need thereof an effective amount of at least one entity chosen from solid-state forms of Compound 9 and solid-state forms of pharmaceutically acceptable salts of Compound 9.

In some embodiments, methods of modulating HIF activity are provided comprising administering to a subject in need thereof an effective amount of at least one entity chosen from solid-state forms of Compound 9 and solid-state forms of pharmaceutically acceptable salts of Compound 9.

In some embodiments, methods for treating cancer are provided comprising administering to a subject in need thereof an effective amount of at least one entity chosen from solid-state forms of Compound 9 and solid-state forms of pharmaceutically acceptable salts of Compound 9.

In some embodiments, the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

More particularly, cancers that may be treated by the compounds, compositions and methods described herein include, but are not limited to, the following: cardiac cancers, including, for example sarcoma, e.g., angiosarcoma, fibrosarcoma, rhabdomyosarcoma, and liposarcoma; myxoma; rhabdomyoma; fibroma; lipoma and teratoma; lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma; gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma; genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma; liver cancers, including, for example, hepatoma, e.g., hepatocellular carcinoma; cholangiocarcinoma; hepatoblastoma; angiosarcoma; hepatocellular adenoma; and hemangioma; bone cancers, including, for example, osteogenic sarcoma (osteosarcoma), fibrosarcoma, malignant fibrous histiocytoma, chondrosarcoma, Ewing's sarcoma family of tumors, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiforme, oligodendroglioma, PNET, schwannoma, retinoblastoma, neuroma and congenital tumors; neural crest derived cancers, e.g. neuroblastoma and cancers of the spinal cord, e.g., neurofibroma, meningioma, glioma, and sarcoma; gynecological cancers, including, for example, cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, mucinous cystadenocarcinoma, unclassified carcinoma, granulosa thecal cell tumors, Sertoli Ley dig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma; hematologic cancers, including, for example cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non Hodgkin's lymphoma (malignant lymphoma) and Waldenstrom's macroglobulinemia; skin cancers, including, for example, malignant melanoma, basal cell carcinoma, squamous cell carcinoma, Kaposi's sarcoma, moles dysplastic nevi, lipoma, angioma, dermatofibroma, keloids, psoriasis; and adrenal gland cancers, including, for example, neuroblastoma, cancers of the eye, including uveal melanoma and retinoblastoma.

Cancers may be solid tumors that may or may not be metastatic. Cancers may also occur, as in leukemia, as a diffuse tissue.

The compounds described herein can also be administered in combination with existing methods of treating cancers, for example by chemotherapy, targeted therapy, immunotherapy, irradiation, or surgery.

Thus, there is further provided a method of treating cancer comprising administering to a subject in need thereof an effective amount of at least one entity chosen from solid-state forms of compound 9 or a solid-state form of pharmaceutically acceptable salts of Compound 9, wherein an effective amount of at least one further cancer chemotherapeutic agent is administered to the subject.

In some embodiments, the further cancer chemotherapeutic agent is chosen from abarelix, aldesleukin, alemtuzumab, alitretinoin, allopurinol, altretamine, anastrozole, arsenic trioxide, asparaginase, axitinib, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, geldanamycin derivatives (e.g., 17-AAG or 17-DMAG), gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, radicicol (Hsp90 inhibitor), rasburicase, rituximab, selumetinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Combinations with immunotherapies could include but not be limited to PD-1 inhibitors (Nivolumab, Pembrolizumab, and Cemiplimab), PDL-1 inhibitors (Atezolimumab, Durvalumab and Avelumab), CTLA-4 inhibitors (Ipilimumab), and/or LAG-3 checkpoint inhibitors (relatlimab).

Further provided herein is a method of treating a hypoxia-related pathology in a subject, comprising administering to the subject an effective amount of at least one entity chosen from solid-state forms of Compound 9 and solid-state forms of pharmaceutically acceptable salts of Compound 9.

In some embodiments, the hypoxia-related pathology is chosen from hypoxemic hypoxia, such as the hypoxia caused by sleep apnea or hypopnea, chronic obstructive pulmonary disease or respiratory arrest, and shunts; anemic hypoxia; hypemic hypoxia, for example, as the result of carbon monoxide poisoning and methaemoglobinaemia; histotoxic hypoxia; and ischemic, or stagnant hypoxia (e.g., cerebral ischemia, ischemic heart disease and intrauterine hypoxia). The term “hypoxia-related pathology” can include a pathology that is caused in part, either directly or indirectly, by conditions of below typical physiological amounts of oxygen. The term “hypoxia-related pathology” also means a pathology caused by a non-hypoxic stimuli. The term includes cancer, cancer metastasis, ischemia, stroke and related conditions, diseases, or syndromes.

In some embodiments, methods for treating noncancerous angiogenic disease are provided comprising administering to a subject in need thereof an effective amount of at least one entity chosen from solid-state forms of Compound 9 and solid-state forms of pharmaceutically acceptable salts of Compound 9.

In some embodiments, the noncancerous angiogenic disease is chosen from atherosclerotic plaque growth and hemorrhage, chronic cystitis, Crohn's disease, diabetic retinopathy, dystrophic epidermolysis bullosa, infantile hemangiomas, intraperitoneal bleeding in endometriosis, macular degeneration, prostate growth in benign prostatic hypertrophy, psoriasis, rheumatoid arthritis, verruca vulgaris, surgical adhesions, keloids, non-cancerous lesions, aneurysms and vascular malformations in the brain, varicose veins, hemorrhoids, and rosacea.

In some embodiments, methods for treating macular degeneration are provided comprising administering to a subject in need thereof an effective amount of at least one entity chosen from solid-state forms of Compound 9 and solid-state forms of pharmaceutically acceptable salts of Compound 9.

In some embodiments, the macular degeneration is chosen from age-related macular degeneration (AMD), dry macular degeneration, wet macular degeneration (e.g., classic choroidal neovascularization and occult choroidal neovascularization), and juvenile macular degeneration or macular dystrophy (e.g., Best's disease, Doyne's honeycomb retinal dystrophy, Sorsby's disease, and Stargardt's disease).

In some embodiments, methods for treating psoriasis are provided comprising administering to a subject in need thereof an effective amount of at least one entity chosen from solid-state forms of Compound 9 and solid-state forms of pharmaceutically acceptable salts of Compound 9.

In some embodiments, methods for treating excessive vascularization are provided comprising administering to a subject in need thereof an effective amount of at least one entity chosen from solid-state forms of Compound 9 and solid-state forms of pharmaceutically acceptable salts of Compound 9.

Tumors are thought to form their own vasculature by different mechanism such as angiogenesis, e.g., the local remodeling of existing vessels; vasculogenesis, the recruitment of endotheial progenitors from the bone-marrow; and transdifferentiation of cancer stem cells into vascular cells.

In some embodiments, the excessive vascularization is chosen from vascularization of the eye such as age-related macular degeneration (AMD) and Diabetic retinopathy.

In some embodiments, the subject is a human. In some embodiments, the subject is a non-human. In some embodiments, the subject is chosen from animals. In some embodiments, the subject is chosen from mammals. In some embodiments, the mammal is a dog or cat.

The at least one entity useful in the practice of the methods described herein may be administered simultaneously, by the same or different routes, or at different times during treatment. The at least one entity may be administered before, along with, or after other medications, including other entities.

The treatment using methods described herein may be carried out for as long a period as necessary, either in a single, uninterrupted session, or in discrete sessions. The treating physician will know how to increase, decrease, or interrupt treatment based on patient response. According to one embodiment, treatment is carried out from about four to about sixteen weeks. The treatment schedule may be repeated as required.

The at least one entity may be administered once daily, twice daily, or three times daily. In some embodiments, the at least one entity is administered once daily. In some embodiments, the at least one entity is administered twice daily. In some embodiments, the at least one entity is administered three times daily.

In some embodiments, 2 mg to 1500 mg of the at least one entity is administered once daily, twice daily, or three times daily. In some embodiments, 2 mg to 1000 mg of the at least one entity is administered once daily, twice daily, or three times daily. In some embodiments, 2 mg to 750 mg of the at least one entity is administered once daily, twice daily, or three times daily. In some embodiments, 2 mg to 500 mg of the at least one entity is administered once daily, twice daily, or three times daily. In some embodiments, 2 mg to 250 mg of the at least one entity is administered once daily, twice daily, or three times daily. In some embodiments, 2 mg to 100 mg of the at least one entity is administered once daily, twice daily, or three times daily. In some embodiments, 50 mg to 1500 mg of the at least one entity is administered once daily, twice daily, or three times daily. In some embodiments, 50 mg to 1000 mg of the at least one entity is administered once daily, twice daily, or three times daily. In some embodiments, 50 mg to 750 mg of the at least one entity is administered once daily, twice daily, or three times daily. In some embodiments, 50 mg to 500 mg of the at least one entity is administered once daily, twice daily, or three times daily. In some embodiments, 50 mg to 250 mg of the at least one entity is administered once daily, twice daily, or three times daily. In some embodiments, 50 mg to 100 mg of the at least one entity is administered once daily, twice daily, or three times daily. In some embodiments, 150 mg to 1500 mg of the at least one entity is administered once daily, twice daily, or three times daily. In some embodiments, 150 mg to 1000 mg of the at least one entity is administered once daily, twice daily, or three times daily. In some embodiments, 150 mg to 750 mg of the at least one entity is administered once daily, twice daily, or three times daily. In some embodiments, 150 mg to 500 mg of the at least one entity is administered once daily, twice daily, or three times daily. In some embodiments, 150 mg to 250 mg of the at least one entity is administered once daily, twice daily, or three times daily.

### Definitions

As used herein, the singular forms of a word also include the plural form, unless the context clearly dictates otherwise; as examples, the terms “a,” “an,” and “the” are understood to be singular or plural. By way of example, “an element” means one or more element.

The terms “at least one” and “one or more” are intended to be synonymous and to refer to no less than one but possibly more, such as one, two, three, etc. For example, the term “at least one entity” refers to one or more entities, such as one entity, two entities, etc.

The term “or” is used herein to mean, and is used interchangeably with, the term “and/or,” unless context clearly indicates otherwise. The term “and/or” as used in a phrase such as “A and/or B” herein is intended to include the following embodiments: “A and B,” “A or B,” “A,” and “B.”.

Likewise, the term “and/or” as used in a phrase such as “A, B, and/or C” is intended to encompass each of the following embodiments: “A, B, and C;” “A, B, or C;” “A or C;” “A or B;” “B or C;” “A and C;” “A and B;” “B and C;” “A” (alone); “B” (alone); and “C” (alone).

The term “DSC” refers to the analytical method of Differential Scanning Calorimetry.

The term “HIF inhibitor” means a compound or pharmaceutically acceptable salt thereof that inhibits the biological activity of any of the HIF factors, e.g., HIF-1, HIF-2, and HIF-3, interferes with the HIF signal transduction pathway, or down regulates expression or availability of HIF in a cell or organism.

The term “hypoxia-related pathology” means a pathology that is caused in part, either directly or indirectly, by conditions of below typical physiological amounts of oxygen. The term includes cancer, cancer metastasis, ischemia, stroke and related conditions, diseases, or syndromes. In some embodiments a hypoxia related pathology is a disorder characterized by abnormal cell proliferation.

The term “hypoxia-related pathology” also means a pathology caused by non-hypoxic stimuli.

The term “non-cancerous angiogenic diseases” refers to non-cancerous diseases or conditions wherein inappropriate angiogenesis is observed as a symptom of the disease.

The term “organism”, “host” or “subject” (as in the subject of the treatment) refers to any living entity comprised of at least one cell. A living organism can be as simple as, for example, a single eukaryotic cell or as complex as a mammal, including a human being. Mammals include, for example, humans; non-human primates, e.g., apes and monkeys; cattle; horses; sheep; rats; mice; pigs; and goats. Non-mammals include, for example, fish and birds.

As used herein, the terms “crystalline form” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD), differential scanning calorimetry (DSC), and/or thermogravimetric analysis (TGA).

As used herein, the term “XRPD” refers to the analytical characterization method of X-ray powder diffraction. XRPD patterns can be recorded at ambient conditions in transmission or reflection geometry using a diffractometer.

As used herein, the terms “X-ray powder diffractogram,” “X-ray powder diffraction pattern,” “XRPD pattern” interchangeably refer to an experimentally obtained pattern plotting signal positions (on the abscissa) versus signal intensities on the ordinate). For an amorphous material, an X-ray powder diffractogram may include one or more broad signals; and for a crystalline material, an X-ray powder diffractogram may include one or more signals, each identified by its angular value as measured in degrees 2θ (° 20), depicted on the abscissa of an X-ray powder diffractogram, which may be expressed as “a signal at . . . degrees two-theta,” “a signal at [a] two-theta value(s) of . . . ” and/or “a signal at at least . . . two-theta value(s) chosen from . . . .”

A “signal” or “peak” as used herein refers to a point in the XRPD pattern where the intensity as measured in counts is at a local maximum. One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

As used herein, “a signal at . . . degrees two-theta,” “a signal at [a] two-theta value[ ] of . . . ” and/or “a signal at at least . . . two-theta value(s) chosen from . . . .” refer to X-ray reflection positions as measured and observed in X-ray powder diffraction experiments (° 20).

The repeatability of the angular values is in the range of±0.2° 2°, i.e., the angular value can be at the recited angular value +0.2 degrees two-theta, the angular value −0.2 degrees two-theta, or any value between those two end points (angular value +0.2 degrees two-theta and angular value −0.2 degrees two-theta).

The terms “signal intensities” and “peak intensities” interchangeably refer to relative signal intensities within a given X-ray powder diffractogram. Factors that can affect the relative signal or peak intensities include sample thickness and preferred orientation (e.g., the crystalline particles are not distributed randomly).

The term “X-ray powder diffractogram having a signal at . . . two-theta values” as used herein refers to an XRPD pattern that contains X-ray reflection positions as measured and observed in X-ray powder diffraction experiments (° 2θ).

As used herein, an X-ray powder diffractogram is “substantially similar to that in [a particular] Figure” when at least 90%, such as at least 95%, at least 98%, or at least 99%, of the signals in the two diffractograms overlap. In determining “substantial similarity,” one of ordinary skill in the art will understand that there may be variation in the intensities and/or signal positions in XRPD diffractograms even for the same crystalline form. Thus, those of ordinary skill in the art will understand that the signal positions in XRPD diffractograms (in degrees two-theta (° 2θ) referred to herein) generally mean that value reported is±0.2 degrees 2θ of the reported value, an art-recognized variance.

As used herein, a crystalline form is “substantially pure” when it accounts for an amount by weight equal to or greater than 90% of the sum of all solid form(s) in a sample as determined by a method in accordance with the art, such as quantitative XRPD. In some embodiments, the solid form is “substantially pure” when it accounts for an amount by weight equal to or greater than 95% of the sum of all solid form(s) in a sample. In some embodiments, the solid form is “substantially pure” when it accounts for an amount by weight equal to or greater than 99% of the sum of all solid form(s) in a sample.

The term “TGA” refers to the analytical method of Thermo Gravimetric (or thermogravimetric) Analysis.

The terms “treating” or “treatment” or “to treat” refer to therapeutic measures (e.g., administration of a medicament(s) to a patient) that cure, slow down, lessen symptoms of, and/or halt progression of the condition. Treatment need not result in a complete cure of the condition; partial inhibition or reduction of the condition being treated is encompassed by this term.

The terms “patient” and “subject” are used synonymously to refer to an adult human individual.

The term “effective amount” as used herein refers to that amount of the compound being administered which will relieve to some extent one or more of the symptoms of the disorder being treated. In reference to cancer or pathologies related to unregulated cell division and/or lack of programmed cell death, a therapeutically effective amount refers to that amount which has the effect of (1) reducing the size of a tumor, (2) inhibiting (that is, slowing to some extent or stopping) aberrant cell division, for example cancer cell division, (3) preventing or reducing the local invasion and distant metastasis of cancer cells, (4) relieving to some extent or eliminating one or more symptoms associated with a pathology related to or caused in part by unregulated or aberrant cellular division, including for example, cancer, (5) prevention of the formation of cancer by application of the compound (like sun screen to protect against cancer), and/or (6) to prevent the chain of events downstream of an initial ischemic condition which leads to the pathology.

As used herein, the terms “prevent” and “preventing” include the prevention of the recurrence, spread or onset. It is not intended that the present disclosure be limited to complete prevention. In some embodiments, the onset is delayed, or the severity is reduced

### EXAMPLES

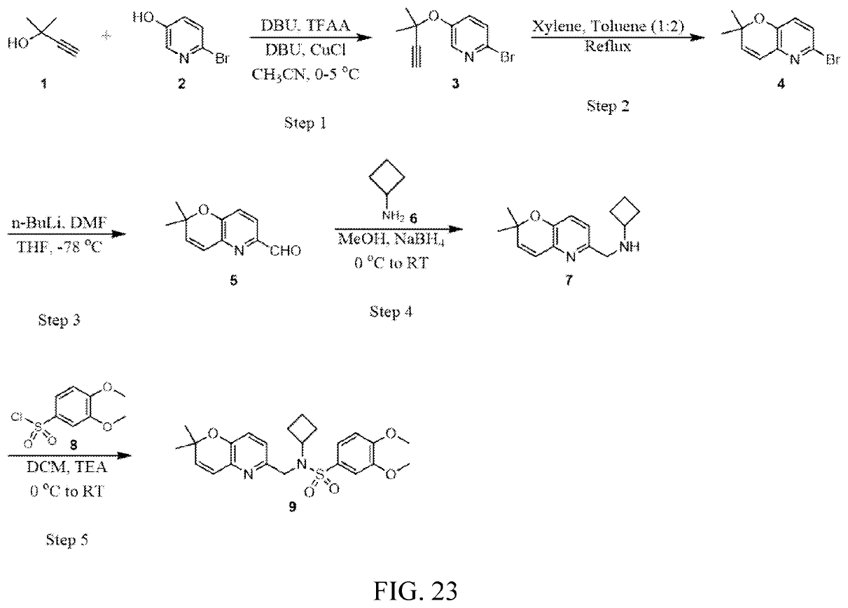

### Example 1: Synthesis of Compound 9 (FIG. 23)

Step-1, 2-Bromo-5-((2-methylbut-3-yn-2-yl)oxy)pyridine, Compound 3: To a stirred solution of 2-methylbut-3-yn-2-ol (compound 1, 116 g, 1.379 mol, 1.2 equiv) in acetonitrile (1500 mL, 7.5 vol) was added DBU (245 g, 1.609 mol, 1.4 equiv) at −5 to 0° C. and the reaction mixture was stirred at room temperature for 30 minutes. Trifluoroacetic anhydride (TFAA) (195 mL, 1.379 mol, 1.2 equiv) was added at −5° C. to 1° C. over a period of 30 minutes. The mixture was stirred at −5° C. to 0° C. for 20 minutes. 6-bromopyridin-3-ol (compound 2, 200 g, 1.149 mol) was taken in a separate flask in acetonitrile (1500 mL) and DBU (297 g, 1.954 mol, 1.7 equiv) was added drop wise at −5° C. to 0° C. over a period of 30 min. Copper chloride (CuCl, 114 mg, 1.149 mmol, 0.001 equiv) was added and the mixture was stirred at 0-5° C. for 30 minutes. The reaction mixture from pot one (which was stored at 0-5° C.) was added slowly over a period of 1 h at −5 to 0° C. and further stirred at 0 to 5° C. for 3 h. The progress of the reaction was monitored by HPLC and TLC. After completion of the reaction, the mixture was poured slowly into cold water (2.0 L) and the product was extracted with ethyl acetate (2 L×3). The combined organic layer was washed with water (2.0 L×3), 1M NaOH solution (500 mL×1) and brine (2.0 L×1). The organic layer was dried over sodium sulphate, filtered, and concentrated under reduced pressure below 50° C. to get crude compound 3 (234.1 g, 85% yield) as a brown liquid.

¹H NMR (CDCl₃): δ 8.28 (d, J=2.8 Hz, 1H), 7.47 (dd, J=2.4 Hz, J=8.8 Hz, 1H), 7.40 (d, J=8.4 Hz, 1H), 2.64 (s, 1H), 1.67 ppm (s, 6H).

Step-2, 6-bromo-2,2-dimethyl-2H-pyrano[3,2-b]pyridine, Compound 4: Compound 3 (234 g, 0.974 mol) was added to toluene (468 mL, 2 vol) and o-xylene (234 mL, 1 vol), and the mixture was refluxed (122° C. internal temperature) for 24-26 h. The progress of the reaction was monitored by HPLC. Note: Further heating is required if the starting material is more than 1% by HPLC. The heating was stopped (3 is <1% by HPLC) and the reaction mixture was cooled to 40-50° C. and dilute with toluene (2 vol). The organic layer was washed with 5% NaOH solution (2.5 vol×1) and water (5 vol×2). Organic layer was dried over sodium sulfate and concentrated under reduced pressure below 60° C. to get 235.0 g of crude product. Purification: n-Heptane (587.5 mL) was added to crude product (235 g) and the mixture was heated to 90° C. for 30 minutes. The mixture was cooled to −50° C. and the supernatant solution containing the product was decanted. n-Heptane (294 mL) was added to the residue, heated to 90° C. for 30 minutes and the clear solution was decanted. The heptane layers were combined, cooled to −10 to −15° C. and stirred for 2 h. The solid precipitated was filtered and dried giving compound 4 (187.7 g, 80.21% yield) as a brownish solid.

¹H NMR (CDCl₃): δ 7.18 (d, J=8.4 Hz, 1H), 6.93 (d, J=8.4 Hz, 1H), 6.48 (d, J=10 Hz, 1H), 5.90 (d, J=10 Hz, 1H), 1.49 ppm (s, 6H).

Step 3, 2,2-Dimethyl-2H-pyrano[3,2-b]pyridine-6-carbaldehyde, Compound 5: Compound 4 (45.5 g, 0.189 mol) was added to a round bottom flask containing anhydrous THE (450 mL) under N₂ atmosphere. The mixture was cooled −78° C. and n-BuLi (98.50 mL, 0.246 mol, 1.3 equiv, 2.5M in n-hexane) was added slowly through cannula at −78° C. to −72° C. The reaction mixture was stirred at −78° C. for 1.5 h until consumption of starting compound 4 to de-brominated intermediate was confirmed. DMF (19.37 g, 0.265 mol, 1.4 equiv) was added slowly with maintenance of the reaction mass temperature between −78° C. and −72° C. and the mixture was stirred −78° C. to −75° C. for 1.5 h until consumption of the de-brominated intermediate was confirmed by HPLC. The reaction was quenched with saturated aqueous ammonium chloride solution (196 mL) at below −65° C. and the reaction mixture was then allowed to warm to room temperature. The product was extracted with ethyl acetate (10 vol×2) and the combined organic layers were washed with brine (5 vol×2). The organic layer was dried over sodium sulphate, filtered, and concentrated giving crude compound 5 (34.8) as brownish semi solid. Crude compound 5 (34.8 g) was purified by silica gel column chromatography eluting with 0-7% ethyl acetate in petroleum ether. Pure fractions were combined and concentrated giving compound 5 (26.2 g, 73.1% yield) as a yellowish solid.

¹H NMR (CDCl₃): δ 9.93 (d, J=0.8 Hz, 1H), 7.76 (d, J=8.4 Hz, 1H), 7.15 (dd, J=0.8 Hz, J=7.6 Hz, 1H), 6.60 (dd, J=0.6 Hz, J=10.2 Hz, 1H), 6.01 (d, J=10 Hz, 1H), 1.53 ppm (s, 6H).

Step-4, N-((2,2-Dimethyl-2H-pyrano[3,2-b]pyridin-6-yl)methyl)cyclobutanamine, Compound 7: Cyclobutylamine (compound 6, 10.33 g, 0.145 mol, 1.1 equiv) was added to a solution of compound 5 (25 g, 0.12 mol) in methanol (250 mL) at below 10° C. over a period of 15 minutes under a N₂ atmosphere. The reaction mixture was allowed to warm to room temperature and was stirred for 5 h. Once the imine formation was complete (as confirmed by ¹H NMR), the reaction mixture was cooled to 10° C. and sodium borohydride (3.99 g, 0.105 mol, 0.8 equiv) was added portion-wise over a period of 20 minutes at 10-15° C. (Note: Mild exotherm was observed during NaBH₄ addition). The mixture was slowly allowed to room temperature and stirred over 2 h and reaction completion was monitored by HPLC. The reaction mixture was quenched with slow addition of cold water (250 ml) at below 10° C. and stirred at room temperature for 30 minutes. The product was extracted with ethyl acetate (250 mL×2). The combined organic layer was washed with water (250 mL×2) and brine (250 mL×1). The organic layer was dried over sodium sulphate, filtered, and concentrated giving crude compound 7 (31.4 g) as a thick brownish liquid. Crude compound 7 (31.4 g) was purified by column chromatography over neutral alumina eluted with 0-3% methanol in DCM. Fractions containing pure material were combined and concentrated giving pure compound 7 (29.5 g, 91.38% yield) as a yellow liquid.

¹H NMR (CDCl₃): δ 7.01 (m, 2H), 6.51 (d, J=10 Hz, 1H), 5.88 (d, J=10 Hz, 1H), 3.73 (s, 2H), 3.38-3.32 (m, 1H), 2.24-2.18 (m, 2H), 1.85-1.63 (m, 5H), 1.49 ppm (s, 6H).

Step-5, N-Cyclobutyl-N-((2,2-dimethyl-2H-pyrano[3,2-b]pyridin-6-yl)methyl)-3,4-di-methoxy benzenesulfonamide, Compound 9: To a solution of compound 7 (28 g, 0.114 mol) in DCM (168 mL, 6 vol) was added triethylamine (26.06 g, 0.257 mol, 2.25 equiv) under nitrogen. The mixture was cooled to 0-5° C. and 3,4-dimethoxybenzenesulfonyl chloride (compound 8, 33.89 g, 0.143 mol, 1.25 equiv) was added in portions at 0-5° C. maintaining the temperature below 10° C. The reaction mixture was allowed to warm to room temperature and was stirred for 16 h. After reaction completion was confirmed by HPLC, the mixture was diluted with DCM (560 mL), washed with cold water (280 mL×2), 10% sodium bicarbonate solution (280 mL×2) and water (280 mL×1). The organic layer was dried over sodium sulphate, filtered, and concentrated under vacuum to get crude product (53 g). Crude compound 9 (53 g) was purified by column chromatography over silica gel by eluting with 0-30% ethyl acetate in petroleum ether. Pure fractions combined and concentrated under vacuum giving pure compound 9 (33.2 g) as an off-white solid. To remove residual DCM in the isolated product, purified compound 9 (33.2 g) was suspended in ethanol (20 vol), heated to 70° C. and stirred for 30 minutes. The resulting clear solution was concentrated under reduced pressure and further dried under high vacuum (1-2 mbar) at 100° C. giving compound 9 (31.6 g, 62.0% yield).

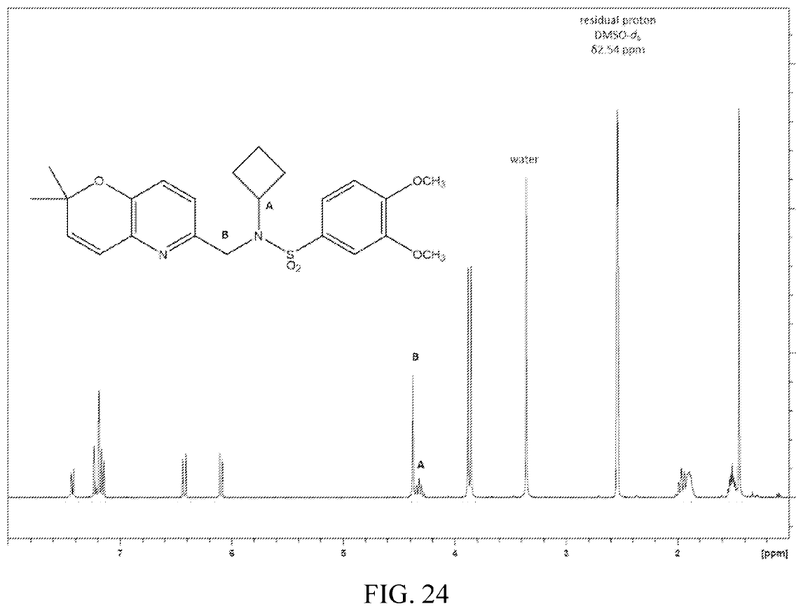

¹H NMR (DMSO-d6): δ 7.42 (d, J=8.4 Hz, 1H), 7.23 (s, 1H), 7.19 (s, 2H), 7.16 (d, J=8.4, 1H), 6.42 (d, J=10.1 Hz, 1H), 6.09 (d, J=10.1 Hz, 1H), 4.37 (s, 2H), 4.32 (m, J=9.6 Hz, 1H), 3.88 (s, 3H), 3.85 (s, 3H), 2.02-1.85 (m, 4H), 1.58-1.45 (m, 2H), 1.45 ppm (s, 6H). See FIG. 24.

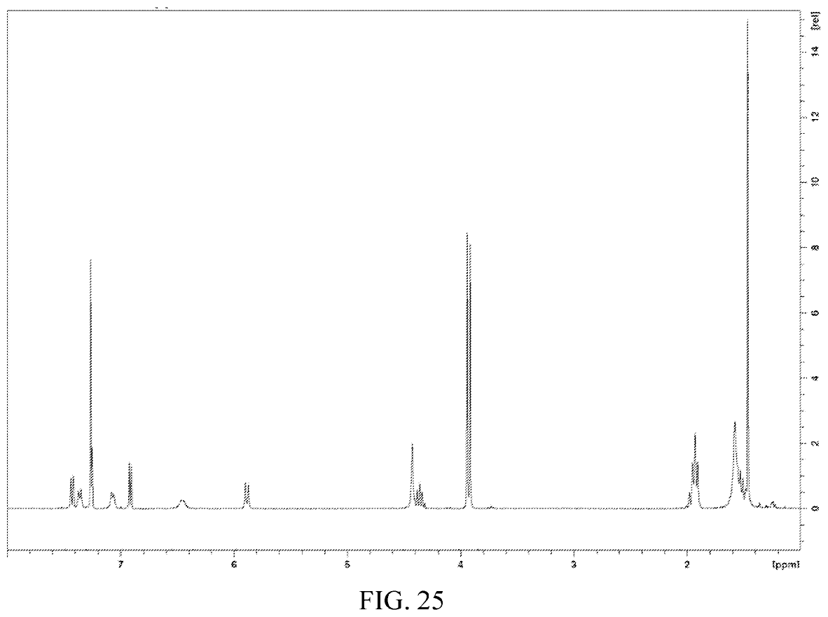

¹H NMR (CDCl₃): δ 7.44 (dd, J=8.4 Hz, 2.2 Hz, 1H), 7.36 (d, J=8.4 Hz, 1H), 7.31-7.21 (m, 2H), 7.07 (d, J=8.4 Hz, 1H), 6.92 (d, J=8.4 Hz, 1H), 6.45 (m, 1H), 4.43 (s, 2H), 4.43-4.28 (m, 1H), 3.94 (s, 3H), 3.92 (s, 3H), 2.00-1.90 (m, 4H), 1.62-1.47 (m, 2H), 1.47 ppm (s, 6H). See FIG. 25.

### Example 2: General Method for XRPD Analysis

X-ray powder diffraction (XRPD) analyses of all but the mesylate salt of compound 9 were obtained using a BRUKER D8 ADVANCE equipped with a Cu radiation source operated at 40 kV and 40 mA (λ=1.54 after Ni filtering) configured in a Bragg-Brentano geometry, equipped with a 90-position AUTO-CHANGER and a silicon strip detector (SSD160-2). A coupled theta-two theta continuous PSD (Position Sensitive Device) fast scan from 4 to 40 (or 80) degrees two theta were collected over approximately 32 minutes (0.5 sec/0.01 step). A sample was placed onto a polished zero-background silicon wafer by gently distributing onto the flat surface and analyzed as a flat plate specimen.

XRPD of the mesylate salt of compound 9 was obtained using a RIGAKU MINIFLEX X-RAY DIFFRACTOMETER equipped with a Cu radiation source operated at 30 kV and 15 mA (λ=1.5401 after Ni filtering). A scan from 5 to 60 degrees two theta were collected over approximately 55 minutes (0.05 degrees/step; 3 sec/step). Samples were evaluated in a manner substantially similar to those used with the BRUKER D8 ADVANCE system above.

### Example 3: General Method for DSC Analysis

Differential scanning calorimetry (DSC) runs were generated on a TA Instruments DSC 2500 equipped with an auto-sampler and RCS90. Typically, 1-3 mg of sample in a Tzero hermetically sealed pan was heated at 10° C. per minute ramp rate from −80° C. or ambient temperature to approximately the degradation onset temperature, as determined by TGA, in T4P mode was used. A purge of dry nitrogen at 50 mL/min was maintained over the sample during the experiment. The instrument control and data analysis were operated under TRIOS software.

### Example 4: General Method for TGA Analysis

Thermogravimetric analysis (TGA) data were collected using a TA Instruments Discovery TGA equipped with an autosampler. Typically, 2-5 mg of sample was placed in a pre-tared, Tzero aluminum pan and automatically loaded for analysis. A 10° C. per minute ramp rate from ambient temperature to 375° C. using a 25 mL/min nitrogen purge. The instrument control and data analysis were operated under TRIOS software.

### Example 5: General Method for DVS Analysis

Samples were analyzed for Dynamic Vapor Sorption using a TA Instruments Q5000SA gravimetric water sorption analyzer. The relative humidity (RH) was adjusted in 10% RH increments between 5-95% (±1% RH) at 25° C. (0.5% ° C.). The mass of a sample was continuously monitored and recorded with respect to RH and time with criteria for mass equilibrium set at a percent mass change <0.0100 for 5 minutes with a time limit of 720 minutes per step. The humidity was controlled by mixing dry and wet nitrogen streams with a total flow rate of 200 mL/min. The instrument control and data analysis were operated under Advantage for Q Series and Universal Analysis software, respectively.

### Example 6: General Method for NMR Analysis

Nuclear magnetic resonance (NMR) data were acquired on Bruker 400 MHz spectrometer at ambient temperature in the specified solvent and the chemical shifts reported in ppm.

### Example 7: Crystal Form Analysis of Compound 9

X-Ray Powder Diffraction: Compound 9 was characterized by XRPD. XRPD patterns evidenced that the compound is crystalline (FIG. 2). The 2 theta and relative intensity values are summarized in Table 1.

TABLE 1
Peak list from powder X-ray powder diffraction
diffractogram of Form A of Compound 9

| Angle | Intensity |
|---|---|
| Degrees 2-Theta ± 0.2) | % |
| 14.1 | 100 |
| 17.2 | 16.5 |
| 17.6 | 30.0 |
| 18.0 | 26.1 |
| 18.4 | 17.1 |
| 20.0 | 17.6 |
| 20.2 | 21.4 |
| 20.6 | 17.1 |
| 22.2 | 16.1 |
| 23.3 | 20.7 |
| 25.9 | 14.5 |

Differential Scanning Calorimetry Analysis—Form A: The DSC thermogram (FIG. 3) shows a melting onset at about 148° C. with a peak at about 150° C.

Thermogravimetric Analysis—Form A: The TGA thermogram (FIG. 3) shows weight loss of about 0.6% w/w from ambient temperature up to about 153° C.

### Example 8: General Protocol for Salt Form Preparation

Approximately 150 mg (0.33 mmol) of Compound 9 was weighed into 1-dram or 20 mL scintillation vials and dissolved in an appropriate solvent. Solid acids were pre-dissolved in DMSO (200 μL) and added over 1-2 minutes to the Compound 9 solutions. Sulfuric acid and hydrochloric acid were introduced over 1-2 minutes as 12.1M, 4M or 1M aqueous solutions. After adding the acids, the resulting solutions were heated with stirring to 50° C. for approximately one hour. MTBE was then added and the mixtures were allowed to cool to room temperature overnight with stirring. Vials were then cooled to 0° C. with stirring for an additional 12 hours. Solids were collected from each vial by filtration and were dried at 30° C. under vacuum for 5-10 hours prior to analysis.

### Example 9: Compound 9 Mesylate Salt

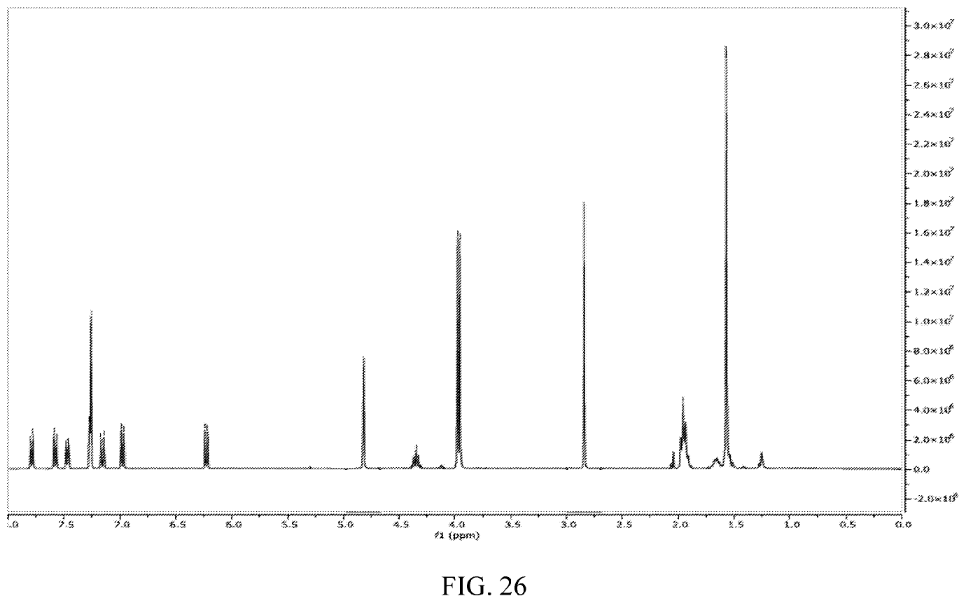

Method of Preparation: The mesylate salt of Compound 9 was prepared in a manner similar to that described in Example 8. ¹H NMR (CDCl₃): δ 7.79 (d, 1H), 7.58 (d, 1H), 7.47 (d, 1H), 7.27 (m, 1H), 7.15 (d, 1H), 6.98 (d, 1H), 6.23 (d, 1H), 4.82 (s, 2H), 4.35 (m, 1H), 3.98 (s, 3H), 3.96 (s, 3H), 2.84 (s, 3H), 2.00-1.85 (m, 4H), 1.70-1.50 (m, 2H), 1.57 ppm (s, 6H). See FIG. 26.

X-Ray Powder Diffraction—Form A: XRPD pattern evidenced that the compound is crystalline (FIG. 4). The 2 theta and relative intensity values are summarized in Table 4.

TABLE 4
Peak list from powder X-ray powder diffraction diffractogram
of Form A of Compound 9 Mesylate Salt

| Angle | Intensity |
|---|---|
| (Degrees 2-Theta ± 0.2) | % |
| 9.3 | 73.6 |
| 17.6 | 32.7 |
| 18.5 | 100 |
| 19.2 | 59.9 |
| 24.7 | 31.5 |

Differential Scanning Calorimetry Analysis—Form A: The DSC thermogram (FIG. 5) shows a melting onset at about 179° C. with a peak at about 180° C.

Thermogravimetric Analysis—Form A: The TGA thermogram (FIG. 6) shows weight loss of about 2% w/w from ambient temperature up to about 200° C.

### Example 10: Compound 9 Hydrochloride Salt

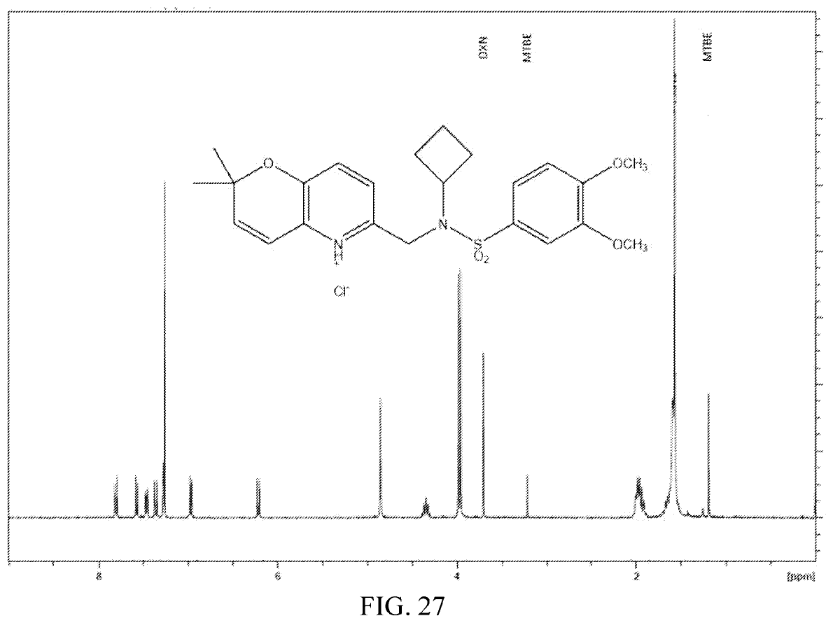

Method of Preparation—Form A: Compound 9 (51.5 mg, 0.12 mmol) was weighed into a 20 mL scintillation vial and dissolved in dioxane (0.50 mL). Hydrochloric acid (1M in water, 35 μL, 0.14 mmol) was added. MTBE (1.10 mL) was added and the mixture was stored in a freezer for 6 days. MTBE (100 μL) was added and the mixture was stored in a freezer for 1 day. MTBE (200 μL) was added and the mixture was stored in a freezer for 1 day. After a total of 8 days in a freezer, white solids (15 mg) were collected by filtration and dried under vacuum. Isolated solids were analyzed for solid-state properties. ¹H NMR (CDCl₃): δ 7.81 (d, J=8.7 Hz, 1H), 7.58 (d, J=8.7 Hz, 1H), 7.46 (d, J=8.4 Hz, 1H), 7.36 (d, J=10.3 Hz, 1H), 7.28 (m, 1H), 6.97 (d, J=8.5 Hz, 1H), 6.22 (d, J=10.3 Hz, 1H), 4.85 (s, 2H), 4.35 (m, 1H), 3.98 (s, 3H), 3.96 (s, 3H), 2.20-1.85 (m, 4H), 1.75-1.56 (m, 2H), 1.56 ppm (s, 6H) See FIG. 27.

X-Ray Powder Diffraction—Form A: XRPD pattern evidenced that the compound is crystalline (FIG. 7). The 2 theta and relative intensity values are summarized in Table 5.

TABLE 5
Peak list from powder X-ray powder diffraction diffractogram
of Form A of Compound 9 Hydrochloride Salt

| Angle | Intensity |
|---|---|
| (Degrees 2-Theta ± 0.2) | % |
| 6.0 | 100 |
| 7.8 | 41.6 |
| 15.1 | 50.0 |
| 15.5 | 47.7 |
| 17.3 | 54.3 |
| 21.5 | 38.5 |

Differential Scanning Calorimetry Analysis—Form A: The DSC thermogram (FIG. 8) shows a transitionary melting onset at about 66° C. with a peak at about 85° C. and a terminal melting onset at about 130° C. with a peak at about 141° C.

Thermogravimetric Analysis—Form A: The TGA thermogram (FIG. 8) shows weight loss of about 13.5% w/w from ambient temperature up to about 155° C.

Method of Preparation—Form B: Compound 9 (151.5 mg, 0.34 mmol) was weighed into a 1-dram vial and dissolved in 25% chloroform in ethyl acetate (0.4 mL). Hydrochloric acid (1M in water, 550 μL, 0.55 mmol, 1.6 equivalents) was added. The resulting mixture was heated to 50° C. for approximately one hour. MTBE (1.50 mL) was added and the resulting mixture was cooled to 0° C. with stirring for an approximately 8 hours. Solids were collected by filtration and were dried at 30° C. under vacuum for 5-10 hours prior to analysis. 120.0 mg of hydrochloride salt were isolated (73% yield).

X-Ray Powder Diffraction—Form B: XRPD pattern evidenced that the compound is crystalline (FIG. 9). The 2 theta and relative intensity values are summarized in Table 6.

TABLE 6
Peak list from powder X-ray powder diffraction diffractogram
of Form B of Compound 9 Hydrochloride Salt

| Angle | Intensity |
|---|---|
| (Degrees 2-Theta ± 0.2) | % |
| 8.7 | 93.5 |
| 9.1 | 29.2 |
| 10.0 | 18.3 |
| 15.8 | 31.6 |
| 17.5 | 24.2 |
| 18.2 | 100 |
| 20.1 | 22.1 |
| 20.7 | 29.8 |
| 21.3 | 34.3 |

Differential Scanning Calorimetry Analysis—Form B: The DSC thermogram (FIG. 10) shows a transitionary melting onset at 160° C. with a peak at about 165° C. and a terminal melting onset at about 183° C. with a peak at about 186° C.

Thermogravimetric Analysis—Form B: The TGA thermogram (FIG. 10) shows weight loss of about 8.9% w/w from ambient temperature up to 169° C.

Method of Preparation—Form C: Compound 9 (161.9 mg, 0.36 mmol) was weighed into a 20 mL scintillation vial and dissolved in 25% chloroform in ethyl acetate (0.4 mL). Hydrochloric acid (1M in water, 550 μL, 0.55 mmol, 1.6 equivalents) was added. The resulting mixture was heated to 50° C. for approximately one hour. MTBE (1.50 mL) was added and the resulting mixture was cooled to room temperature with stirring for approximately 8 hours. Solids were collected by filtration and were dried at 30° C. under vacuum for 5-10 hours prior to analysis. 96.2 mg of hydrochloride salt were isolated (55% yield).

X-Ray Powder Diffraction—Form C: XRPD pattern evidenced that the compound is crystalline (FIG. 11). The 2 theta and relative intensity values are summarized in Table 7.

TABLE 7
Peak list from powder X-ray powder diffraction diffractogram
of Form C of Compound 9 Hydrochloride Salt

| Angle | Intensity |
|---|---|
| Degrees 2-Theta ± 0.2) | % |
| 10.2 | 29.9 |
| 11.6 | 44.3 |
| 12.0 | 85.8 |
| 13.7 | 33.7 |
| 16.6 | 92.7 |
| 17.1 | 74.4 |
| 23.3 | 100 |

Differential Scanning Calorimetry Analysis—Form C: The DSC thermogram (FIG. 12) shows a transitionary melting onset at about 132° C. with a peak at about 137° C. and a terminal melting onset at about 181° C. with a peak at about 188° C.

Thermogravimetric Analysis—Form C: The TGA thermogram (FIG. 12) shows weight loss of about 7.8% w/w from ambient temperature up to 181° C.

### Example 11: Compound 9 Sulfate Salt

Method of Preparation: Compound 9 (153.1 mg, 0.34 mmol) was weighed into a 1-dram vial and dissolved in dioxane (1.1 mL). Sulfuric acid (4M in water, 150 μL, 0.45 mmol, 1.32 equivalents) was added. The resulting mixture was heated to 50° C. for approximately one hour. MTBE (1.20 mL) was added and the resulting mixture was allowed to cool to room temperature overnight with stirring. The vial was then cooled to 0° C. with stirring for an additional 12 hours. Solids were collected by filtration and were dried at 30° C. under vacuum for 5-10 hours prior to analysis. 154.5 mg of sulfate salt were isolated (83% yield).

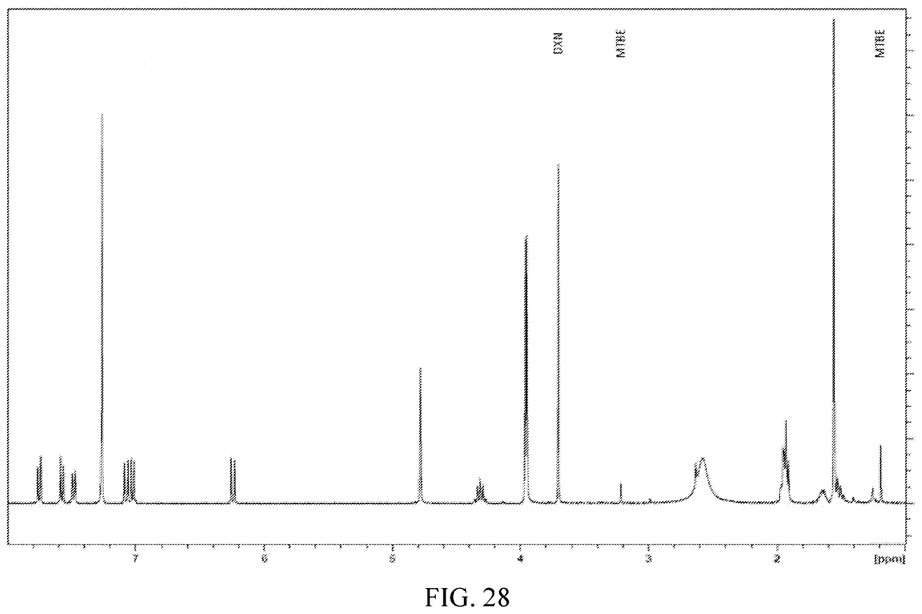

¹H NMR (CDCl₃): δ 7.75 (d, J=8.6 Hz, 1H), 7.57 (d, J=8.6 Hz, 1H), 7.47 (d, J=8.5 Hz, 1H), 7.26 (s, 1H), 7.07 (d, J=10.3 Hz, 1H), 7.02 (d, J=8.5 Hz, 1H), 6.24 (d, J=10.3 Hz, 1H), 4.78 (s, 2H), 4.35 (m, 1H), 3.96 (s, 3H), 3.95 (s, 3H), 2.00-1.90 (m, 4H), 1.70-1.60 (m, 1H), 1.56 (s, 6H), 1.56-1.45 ppm (m, 1H). See FIG. 28.

X-Ray Powder Diffraction—Form A (pre-vacuum, pre-DVS): XRPD pattern evidenced that the compound is crystalline (FIG. 13). The 2 theta and relative intensity values are summarized in Table 8.

TABLE 8
Peak list from powder X-ray powder diffraction diffractogram
of Form A of Compound 9 Sulfate Salt

| Angle | Intensity |
|---|---|
| (Degrees 2-Theta ± 0.2) | % |
| 5.7 | 84.6 |
| 8.6 | 32.6 |
| 8.8 | 16.9 |
| 10.3 | 24.6 |
| 15.3 | 21.5 |
| 16.4 | 80.0 |
| 17.1 | 100 |
| 18.2 | 44.9 |

Differential Scanning Calorimetry Analysis—Form A: The DSC thermogram (FIG. 14) shows a terminal melting onset at about 177° C. with a peak at about 194° C.

Thermogravimetric Analysis—Form A: The TGA thermogram (FIG. 14) shows weight loss of about 9.9% w/w from ambient temperature up to 130° C.

DVS Analysis—Form A: The first adsorption cycle of the DVS experiment displayed a slight adsorption of 0.2% at 55% RH followed by a significant desorption of 12.9% at 85% RH. Afterwards there was a slight adsorption of 0.7% at 95% RH and then followed by a 1.4% mass loss during desorption. The second desorption/adsorption cycle showed very little hysteresis with an average of 1.4%. See FIG. 15.

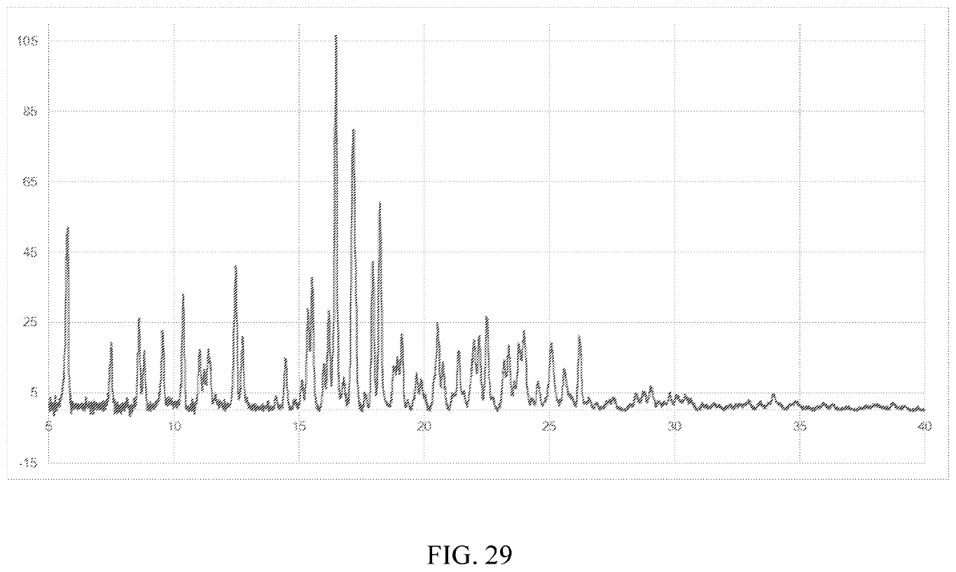

X-Ray Powder Diffraction—Form A/B (post-vacuum): XRPD pattern evidenced that the compound is crystalline (FIG. 29). The 2 theta and relative intensity values are summarized in Table 9.

TABLE 9
Peak list from powder X-ray powder diffraction diffractogram
of Form A/B of Compound 9 Sulfate Salt

| Angle | Intensity |
|---|---|
| (Degrees 2-Theta ± 0.2) | % |
| 5.7 | 48.8 |
| 7.5 | 18.0 |
| 8.6 | 24.6 |
| 8.8 | 16.1 |
| 9.6 | 21.4 |
| 10.3 | 30.8 |
| 11.0 | 16.2 |
| 11.4 | 16.2 |
| 12.5 | 37.8 |
| 12.7 | 19.0 |
| 15.3 | 27.1 |
| 15.5 | 35.5 |
| 16.5 | 100 |
| 17.2 | 74.6 |
| 18.0 | 39.5 |
| 18.2 | 55.5 |
| 20.5 | 23.5 |
| 22.5 | 25.1 |
| 26.2 | 19.4 |

X-Ray Powder Diffraction—Form B (post-DVS): XRPD pattern evidenced that the compound is crystalline (FIG. 16). The 2 theta and relative intensity values are summarized in Table 10.

TABLE 10
Peak list from powder X-ray powder diffraction diffractogram
of Form B of Compound 9 Sulfate Salt

| Angle | Intensity |
|---|---|
| (Degrees 2-Theta ± 0.2) | % |
| 7.4 | 49.0 |
| 9.5 | 56.5 |
| 11.4 | 32.9 |
| 12.4 | 66.8 |
| 12.7 | 40.4 |
| 15.5 | 95.3 |
| 16.2 | 64.5 |
| 17.3 | 40.7 |
| 18.0 | 100 |
| 19.1 | 47.2 |
| 22.5 | 50.3 |
| 23.8 | 47.1 |
| 26.2 | 58.9 |

### Example 12: Compound 9 Tosylate Salt

Method of Preparation: Compound 9 (153.3 mg, 0.34 mmol) was weighed into a 20 mL scintillation vial and dissolved in 2-methyl THE (3.6 mL). para-Toluenesulfonic acid (86 mg, 0.45 mmole, 1.31 equivalents) was dissolved in DMSO (200 μL) and added to the Compound 9 solution. The resulting mixture was heated to 50° C. for approximately one hour. MTBE (2.60 mL) was added and the resulting mixture was allowed to cool to room temperature overnight with stirring. The vial was then cooled to 0° C. with stirring for an additional 12 hours. Solids were collected by filtration and were dried at 30° C. under vacuum for 5-10 hours prior to analysis. 166.8 mg of tosylate salt were isolated (78% yield).

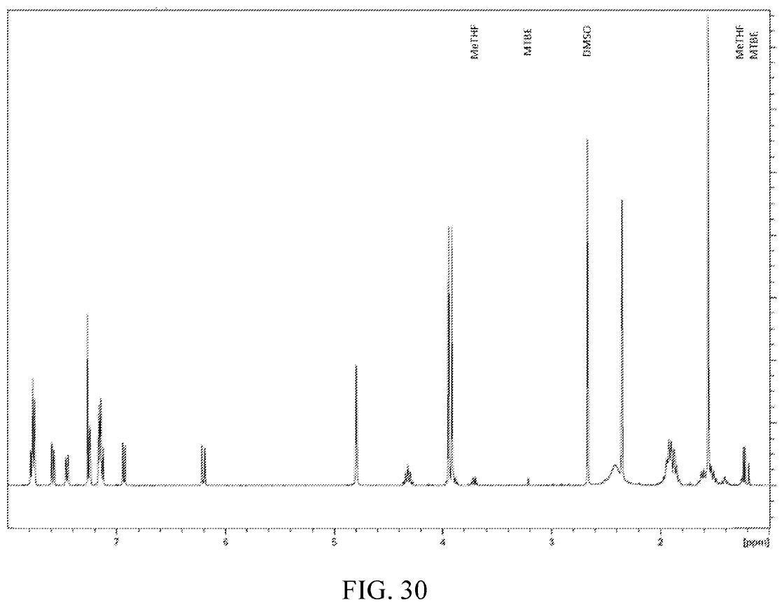

¹H NMR (CDCl₃): δ 7.77-7.75 (m, 3H), 7.58 (d, J=8.7 Hz, 1H), 7.45 (d, J=8.4 Hz, 1H), 7.25 (s, 1H), 7.16-7.12 (m, 3H), 6.93 (d, J=8.5 Hz, 1H), 6.20 (d, J=10.3 Hz, 1H), 4.80 (s, 2H), 4.32 (m, 1H), 3.95 (s, 3H), 3.92 (s, 3H), 2.35 (s, 3H), 2.00-1.80 (m, 4H), 1.65-1.56 (m, 1H), 1.56 (s, 6H), 1.56-1.50 ppm (m, 1H). See FIG. 30.

X-Ray Powder Diffraction—Form A: XRPD pattern evidenced that the compound is crystalline (FIG. 17). The 2 theta and relative intensity values are summarized in Table 11.

TABLE 11
Peak list from powder X-ray powder diffraction diffractogram
of Form A of Compound 9 Tosylate Salt

| Angle | Intensity |
|---|---|
| (Degrees 2-Theta ± 0.2) | % |
| 6.4 | 42.8 |
| 7.3 | 100 |
| 9.3 | 94.3 |
| 17.9 | 61.3 |
| 19.5 | 49.1 |
| 21.0 | 31.9 |
| 21.4 | 45.8 |
| 27.3 | 27.7 |

Differential Scanning Calorimetry Analysis: The DSC thermogram (FIG. 18) shows a melting onset at about 76° C. with a peak at about 91° C.

Thermogravimetric Analysis: The TGA thermogram (FIG. 18) shows weight loss of about 4.8% w/w from ambient temperature up to 111° C. and about 8.3% w/w from ambient temperature up to 154° C.

DVS Analysis: Two DVS cycles were performed measuring mass change over time as relative humidity (RH) was cycled from 5% to 95% and then from 95% to 5%. During the first cycle, a mass increase 0.83% was observed at 55% RH followed by a net mass decrease of 3.88% as the RH was increased to 75%. A subsequent net mass increase of 4.3% was noted when the RH was increased from 75% to 95% resulting in a total net mass increase of 1.25%. When the RH was reduced from 95% to 5% in the first cycle, a net mass decrease of 9.03% was observed. These observations are sharply different from the second DVS cycle where a net mass increase of 13.8% was observed as the RH increased from 5% to 95%. When the RH was reduced from 95% to 5%, a net mass decrease of 13.92% was observed. The post-DVS material is most likely a different solid state form. See FIG. 19.

### Example 13: Compound 9 Napthalene-1,5-Disulfonic Acid Hemi-Salt

Method of Preparation: Compound 9 (152.9 mg, 0.34 mmol) was weighed into a 20 mL scintillation vial and dissolved in 2-methyl THE (3.6 mL). Naphthalene-1,5-disulfonic acid tetrahydrate (65 mg, 0.179 mmole, 0.52 equivalents) was dissolved in DMSO (200 μL) and added to the Compound 9 solution. The resulting mixture was heated to 50° C. for approximately one hour. The resulting mixture was allowed to cool to room temperature overnight with stirring. The vial was then cooled to 0° C. with stirring for an additional 12 hours. Solids were collected by filtration and were dried at 30° C. under vacuum for 5-10 hours prior to analysis. 152.1 mg of napadisylate hemi-salt were isolated (60% yield).

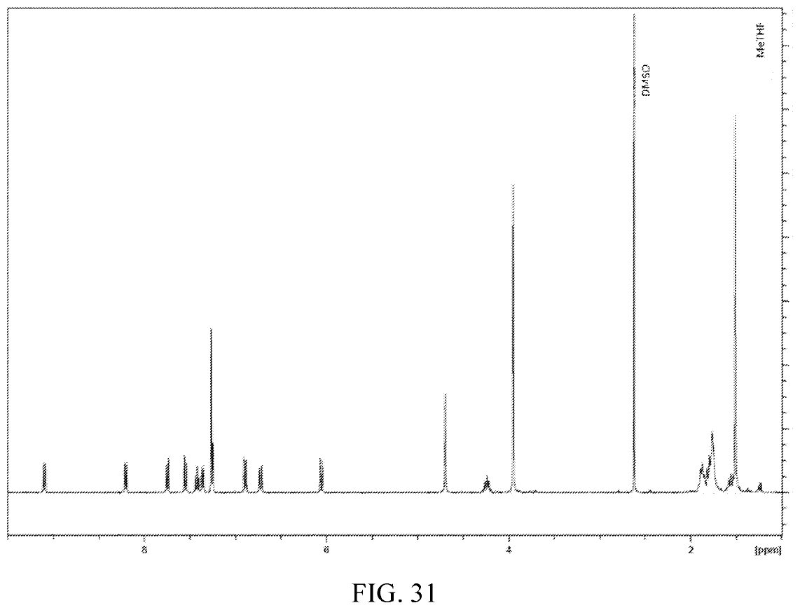

¹H NMR (CDCl₃): δ 9.10 (d, J=8.6 Hz, 2H), 8.20 (d, J=7.1 Hz, 2H), 7.74 (d, J=8.7 Hz, 2H), 7.55 (d, J=8.7 Hz, 2H), 7.42 (m, 2H), 7.36 (d, J=8.5 Hz, 2H), 7.25 (s, 2H), 6.89 (d, J=8.5 Hz, 2H), 6.72 (d, J=10.3 Hz, 2H), 6.05 (d, J=10.3 Hz, 2H), 4.70 (s, 4H), 4.24 (m, 2H), 3.95 (s, 12H), 2.00-1.70 (m, 8H), 1.70-1.51 (m, 4H), 1.51 ppm (s, 12H). See FIG. 31.

X-Ray Powder Diffraction—Form A: XRPD pattern evidenced that the compound is crystalline (FIG. 20). The 2 theta and relative intensity values are summarized in Table 11.

TABLE 11
Peak list from powder X-ray powder diffraction diffractogram of
Form A of Compound 9 Napthalene-1,5-Disulfonic Acid Hemi-Salt

| Angle | Intensity |
|---|---|
| (Degrees 2-Theta ± 0.2) | % |
| 9.9 | 100 |
| 10.5 | 57.4 |
| 12.4 | 37.8 |
| 13.1 | 48.0 |
| 15.1 | 41.3 |
| 15.8 | 53.3 |
| 17.5 | 49.3 |
| 17.8 | 43.2 |
| 19.0 | 29.2 |
| 19.7 | 29.5 |
| 20.3 | 40.4 |
| 21.0 | 54.8 |
| 22.2 | 53.0 |
| 28.2 | 53.1 |

Differential Scanning Calorimetry Analysis—Form A: The DSC thermogram (FIG. 21) shows a melting onset at 136° C. with a peak at 140° C.

Thermogravimetric Analysis—Form A: The TGA thermogram (FIG. 21) shows weight loss of about 6.5% w/w from ambient temperature up to 154° C.

DVS Analysis—Form A: The DVS curve (FIG. 22) demonstrated that the average water sorbed or desorbed for this material is 5.6±0.7%. This material should be considered hygroscopic at 95% RH (<15% and ≥2%, Ph. Eur. 9.0). XRPD results post DVS were comparable to the pre-DVS XRPD suggesting no solid form change.

### REFERENCES

Dong L, You S, Zhang Q, Osuka S, Devi N S, Kaluz S, et al. Arylsulfonamide 64B Inhibits Hypoxia/HIF-Induced Expression of c-Met and CXCR4 and Reduces Primary Tumor Growth and Metastasis of Uveal Melanoma. Clin Cancer Res. 2019; 25(7):2206-18. Epub 2018/12/20. doi: 10.1158/1078-0432.CCR-18-1368. PubMed PMID: 30563937. (“Dong et al. 2019”).
Ferguson J H, De Los Santos Z, Devi S N, Kaluz S, Van Meir E G, Zingales S K, et al. Design and synthesis of benzopyran-based inhibitors of the hypoxia-inducible factor-1 pathway with improved water solubility. J Enzyme Inhib Med Chem. 2017; 32(1):992-1001. doi: 10.1080/14756366.2017.1347784. PubMed PMID: 28766956. (“Ferguson et. al. 2017-A.”)
Ferguson J, De Los Santos Z, Devi N, Van Meir E, Zingales S, Wang B. Examining the structure-activity relationship of benzopyran-based inhibitors of the hypoxia inducible factor-1 pathway. Bioorg Med Chem Lett. 2017; 27(8):1731-6. Epub 2017/03/01. doi: 10.1016/j.bmcl.2017.02.073. PubMed PMID: 28285917. (“Ferguson et al. 2017-B”).
Huang L E, Bunn H F. Hypoxia-inducible factor and its biomedical relevance. J Biol Chem. 2003; 278(22):19575-8. Epub 2003/03/14. doi: 10.1074/jbc.R200030200. PubMed PMID: 12639949. (“Huang et al. 2003”).
Kim Y J, Kaluz S, Mehta A, Weinert E, Rivera S, Van Meir E G. Purifying Properly Folded Cysteine-rich, Zinc Finger Containing Recombinant Proteins for Structural Drug Targeting Studies: the CH1 Domain of p300 as a Case Example. Bio Protoc. 2017; 7(17). doi: 10.21769/BioProtoc.2537. PubMed PMID: 28966947. (“Kim et al. 2017”).
Kaelin W G. The von Hippel-Lindau tumour suppressor protein: 02 sensing and cancer. Nat Rev Cancer. 2008; 8(11):865-73. Epub 2008/10/16. doi: 10.1038/nrc2502. PubMed PMID: 18923434. (“Kaelin et al. 2008”).
Mooring S R, Jin H, Devi N S, Jabbar A A, Kaluz S, Liu Y, et al. Design and synthesis of novel small molecule inhibitors of the hypoxia inducible factor pathway. J Med Chem. 2011; 54(24):8471-89. Epub 2011/11/23. doi: 10.1021/jm201018g. PubMed PMID: 22032632. (“Mooring et al. 2011”).
Mun J, Jabbar A A, Devi N S, Liu Y, Van Meir E G, Goodman M M. Structure-activity relationship of 2,2-dimethyl-2H-chromene based arylsulfonamide analogs of 3,4-dimethoxy-N-[(2,2-dimethyl-2H-chromen-6-yl)methyl]-N-phenylbenzenesulfonamide, a novel small molecule hypoxia inducible factor-1 (HIF-1) pathway inhibitor and anti-cancer agent. Bioorg Med Chem. 2012; 20(14):4590-7. Epub 2012/05/14. doi: 10.1016/j.bmc.2012.04.064. PubMed PMID: 22682301. (“Mun et al. 2012”).
Razafinjatovo, C, Bihr, S, Mischo, A, Vogl, U, Schmidinger, M, Moch, H, and Schraml, P. Characterization of VHL missense mutations in sporadic clear cell renal cell carcinoma: hotspots, affected binding domains, functional impact on pVHL and therapeutic Relevance. BMC Cancer (2016) 16:638, DOI 10.1186/s12885-016-2688-0. (“Razafinjatovo et al. 2016”).
Shi Q, Yin S, Kaluz S, Ni N, Devi N S, Mun J, et al. Binding Model for the Interaction of Anticancer Arylsulfonamides with the p300 Transcription Cofactor. ACS Med Chem Lett. 2012; 3(8):620-5. Epub 2012/06/21. doi: 10.1021/m1300042k. PubMed PMID: 24936238. (“Shi et al. 2012”).
Tan C, de Noronha R G, Devi N S, Jabbar A A, Kaluz S, Liu Y, et al. Sulfonamides as a new scaffold for hypoxia inducible factor pathway inhibitors. Bioorg Med Chem Lett. 2011; 21(18):5528-32. Epub 2011/06/28. doi: 10.1016/j.bmcl.2011.06.099. PubMed PMID: 21831638. (“Tan et al. 2011”).
Wenger R H, Stiehl D P, Camenisch G. Integration of oxygen signaling at the consensus HRE. Sci STKE. 2005; 2005(306):re12. Epub 2005/10/18. doi: 10.1126/stke.3062005re12. PubMed PMID: 16234508. (“Wenger et al. 2005”).
Yin S, Kaluz S, Devi N S, Jabbar A A, de Noronha R G, Mun J, et al. Arylsulfonamide KCN1 inhibits in vivo glioma growth and interferes with HIF signaling by disrupting HIF-1α interaction with cofactors p300/CBP. Clin Cancer Res. 2012; 18(24):6623-33. Epub 2012/08/24. doi: 10.1158/1078-0432.CCR-12-0861. PubMed PMID: 22923450. (“Yin et al. 2012”).

What is claimed is:

1. Form A of Compound 9:

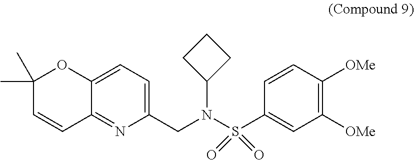 [SMILES: COc1ccc(S(=O)(=O)N(Cc2ccc3c(n2)C=CC(C)(C)O3)C2CCC2)cc1OC]

2. Form A of Compound 9 according to claim 1, characterized by an X-ray powder diffractogram substantially similar to that in FIG. 2.

3. Form A of Compound 9 according to claim 1, characterized by an X-ray diffractogram having a signal at 14.1±0.2, 17.6±0.2, 18.0±0.2, 20.2±0.2, and 23.3±0.2 two-theta.

4. Form A of Compound 9 according to claim 1, characterized by an X-ray powder diffractogram having a signal at at least two two-theta values chosen from 14.1±0.2, 17.6±0.2, 18.0±0.2, 20.2±0.2, and 23.3±0.2.

5. Form A of Compound 9 according to claim 1, characterized by an X-ray powder diffractogram having a signal at at least three two-theta values chosen from 14.1±0.2, 17.6±0.2, 18.0±0.2, 20.2±0.2, and 23.3±0.2.

6. Form A of Compound 9 according to claim 1, characterized by an X-ray powder diffractogram having a signal at at least four two-theta values chosen from 14.1±0.2, 17.6±0.2, 18.0±0.2, 20.2±0.2, and 23.3±0.2.

7. Form A of Compound 9 according to claim 1, characterized by a DSC substantially similar to that in FIG. 3.

8. Form A of Compound 9 according to claim 1, characterized by a DSC having a melting onset at about 148° C. and/or a peak at about 150° C.

9. Form A of Compound 9 according to claim 1, characterized by a DSC having a peak in a range of 147° C. to 151° C.

10. Form A of Compound 9 according to claim 1, characterized by a TGA substantially similar to that in FIG. 3.

11. Form A of Compound 9 according to claim 1, characterized by a TGA showing a weight loss of about 0.6% w/w from ambient temperature up to 153° C.

12. A pharmaceutical composition comprising Form A of Compound 9 according to any one of claims 1 to 11 and a pharmaceutically acceptable carrier.

13. A method of treating a hypoxia-related pathology comprising administering to a subject in need thereof an effective amount of Form A of Compound 9 according to any one of claims 1 to 11 or a pharmaceutical composition according to claim 12.

14. A method of modulating HIF activity comprising administering to a subject in need thereof an effective amount of Form A of Compound 9 according to any one of claims 1 to 11 or a pharmaceutical composition according to claim 12.

15. A method of treating cancer comprising administering to a subject in need thereof an effective amount of Form A of Compound 9 according to any one of claims 1 to 11 or a pharmaceutical composition according to claim 12.

16. The method according to claim 15, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

17. A solid-state form of compound 9: 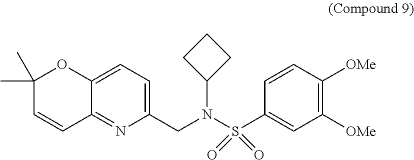 [SMILES: COc1ccc(S(=O)(=O)N(Cc2ccc3c(n2)C=CC(C)(C)O3)C2CCC2)cc1OC]
wherein the solid-state form of compound 9 comprises at least Form A.

18. The solid-state form according to claim 17, wherein the solid-state form of compound 9 consists essentially of Form A.

19. The solid-state form according to claim 17, wherein the solid-state form of compound 9 comprises at least 90% Form A.

20. The solid-state form according to claim 17, wherein the solid-state form of compound 9 comprises at least 95% Form A.

21. The solid-state form according to claim 17, wherein the solid-state form of compound 9 comprises at least 98% Form A.

22. The solid-state form according to any one of claims 17-21, wherein Form A of Compound 9 is characterized by an X-ray powder diffractogram substantially similar to that in FIG. 2.

23. The solid-state form according to any one of claims 17-21, wherein Form A of Compound 9 is characterized by an X-ray diffractogram having a signal at 14.1±0.2, 17.6±0.2, 18.0±0.2, 20.2±0.2, and 23.3±0.2 two-theta.

24. The solid-state form according to any one of claims 17-21, wherein Form A of Compound 9 is characterized by an X-ray powder diffractogram having a signal at at least two two-theta values chosen from 14.1±0.2, 17.6±0.2, 18.0±0.2, 20.2±0.2, and 23.3±0.2.

25. The solid-state form according to any one of claims 17-21, wherein Form A of Compound 9 is characterized by an X-ray powder diffractogram having a signal at at least three two-theta values chosen from 14.1±0.2, 17.6±0.2, 18.0±0.2, 20.2±0.2, and 23.3±0.2.

26. The solid-state form according to any one of claims 17-21, wherein Form A of Compound 9 is characterized by an X-ray powder diffractogram having a signal at at least four two-theta values chosen from 14.1±0.2, 17.6±0.2, 18.0±0.2, 20.2±0.2, and 23.3±0.2.

27. The solid-state form according to any one of claims 17-21, wherein Form A of Compound 9 is characterized by a DSC substantially similar to that in FIG. 3.

28. The solid-state form according to any one of claims 17-21, wherein Form A of Compound 9 is characterized by a DSC having a melting onset at about 148° C. and/or a peak at about 150° C.

29. The solid-state form according to any one of claims 17-21, wherein Form A of Compound 9 is characterized by a DSC having a peak in a range of 147° C. to 151° C.

30. The solid-state form according to any one of claims 17-21, wherein Form A of Compound 9 is characterized by a TGA substantially similar to that in FIG. 3.

31. The solid-state form according to any one of claims 17-21, wherein Form A of Compound 9 is characterized by a TGA showing a weight loss of about 0.6% w/w from ambient temperature up to 153° C.

32. A pharmaceutical composition comprising the solid-state form of Compound 9 according to any one of claims 17 to 31 and a pharmaceutically acceptable carrier.

33. A method of treating a hypoxia-related pathology comprising administering to a subject in need thereof an effective amount of Form A of Compound 9 according to any one of claims 17 to 31 or a pharmaceutical composition according to claim 32.

34. A method of modulating HIF activity comprising administering to a subject in need thereof an effective amount of Form A of Compound 9 according to any one of claims 17 to 31 or a pharmaceutical composition according to claim 32.

35. A method of treating cancer comprising administering to a subject in need thereof an effective amount of Form A of Compound 9 according to any one of claims 17 to 31 or a pharmaceutical composition according to claim 32.

36. The method according to claim 35, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

37. Form A of Compound 9 hydrochloride salt:

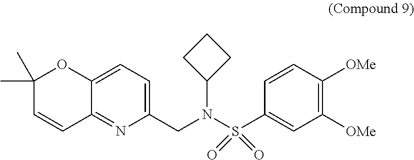 [SMILES: COc1ccc(S(=O)(=O)N(Cc2ccc3c(n2)C=CC(C)(C)O3)C2CCC2)cc1OC]

38. Form A of Compound 9 hydrochloride salt according to claim 37, characterized by an X-ray powder diffractogram substantially similar to that in FIG. 7.

39. Form A of Compound 9 hydrochloride salt according to claim 37, characterized by an X-ray diffractogram having a signal at 6.0±0.2, 7.8±0.2, 15.1±0.2, 15.5±0.2, 17.3±0.2, and 21.5±0.2 two-theta.

40. Form A of Compound 9 hydrochloride salt according to claim 37, characterized by an X-ray powder diffractogram having a signal at at least two two-theta values chosen from 6.0±0.2, 7.8±0.2, 15.1±0.2, 15.5±0.2, 17.3±0.2, and 21.5±0.2.

41. Form A of Compound 9 hydrochloride salt according to claim 37, characterized by an X-ray powder diffractogram having a signal at at least three two-theta values chosen from 6.0±0.2, 7.8±0.2, 15.1±0.2, 15.5±0.2, 17.3±0.2, and 21.5±0.2.

42. Form A of Compound 9 hydrochloride salt according to claim 37, characterized by an X-ray powder diffractogram having a signal at at least four two-theta values chosen from 6.0±0.2, 7.8±0.2, 15.1±0.2, 15.5±0.2, 17.3±0.2, and 21.5±0.2.

43. Form A of Compound 9 hydrochloride salt according to claim 37, characterized by a DSC substantially similar to that in FIG. 8.

44. Form A of Compound 9 hydrochloride salt according to claim 37, characterized by a DSC having a transitionary melting onset at about 66° C. and/or a peak at about 85° C.

45. Form A of Compound 9 hydrochloride salt according to claim 37, characterized by a DSC having a terminal melting onset at about 130° C. and/or a peak at about 141° C.

46. Form A of Compound 9 hydrochloride salt according to claim 37, characterized by a DSC having a peak in a range of 129° C. to 142° C.

47. Form A of Compound 9 hydrochloride salt according to claim 37, characterized by a TGA substantially similar to that in FIG. 8.

48. Form A of Compound 9 hydrochloride salt according to claim 37, characterized by a TGA showing a weight loss of about 13.5% w/w from ambient temperature up to 155° C.

49. A pharmaceutical composition comprising Form A of Compound 9 hydrochloride salt according to any one of claims 37 to 48 and a pharmaceutically acceptable carrier.

50. A method of treating a hypoxia-related pathology comprising administering to a subject in need thereof an effective amount of Form A of Compound 9 hydrochloride salt according to any one of claims 37 to 48 or a pharmaceutical composition according to claim 49.

51. A method of modulating HIF activity comprising administering to a subject in need thereof an effective amount of Form A of Compound 9 hydrochloride salt according to any one of claims 37 to 48 or a pharmaceutical composition according to claim 49.

52. A method of treating cancer comprising administering to a subject in need thereof an effective amount of Form A of Compound 9 hydrochloride salt according to any one of claims 37 to 48 or a pharmaceutical composition according to claim 49.

53. The method according to claim 52, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

54. A hydrochloride salt of compound 9: 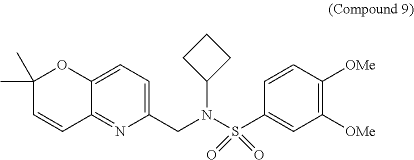 [SMILES: COc1ccc(S(=O)(=O)N(Cc2ccc3c(n2)C=CC(C)(C)O3)C2CCC2)cc1OC]
wherein hydrochloride salt of compound 9 comprises at least Form A.

55. The hydrochloride salt according to claim 54, wherein the hydrochloride salt of compound 9 consists essentially of Form A.

56. The hydrochloride salt according to claim 54, wherein the hydrochloride salt of compound 9 comprises at least 90% Form A.

57. The hydrochloride salt according to claim 54, wherein the hydrochloride salt of compound 9 comprises at least 95% Form A.

58. The hydrochloride salt according to claim 54, wherein the hydrochloride salt of compound 9 comprises at least 98% Form A.

59. The hydrochloride salt according to any one of claims 54-58, wherein Form A of Compound 9 hydrochloride salt is characterized by an X-ray powder diffractogram substantially similar to that in FIG. 7.

60. The hydrochloride salt according to any one of claims 54-58, wherein Form A of Compound 9 hydrochloride salt is characterized by an X-ray diffractogram having a signal at 6.0±0.2, 7.8±0.2, 15.1±0.2, 15.5±0.2, 17.3±0.2, and 21.5±0.2 two-theta.

61. The hydrochloride salt according to any one of claims 54-58, wherein Form A of Compound 9 hydrochloride salt is characterized by an X-ray powder diffractogram having a signal at at least two two-theta values chosen from 6.0±0.2, 7.8±0.2, 15.1±0.2, 15.5±0.2, 17.3±0.2, and 21.5±0.2.

62. The hydrochloride salt according to any one of claims 54-58, wherein Form A of Compound 9 hydrochloride salt is characterized by an X-ray powder diffractogram having a signal at at least three two-theta values chosen from 6.0±0.2, 7.8±0.2, 15.1±0.2, 15.5±0.2, 17.3±0.2, and 21.5±0.2.

63. The hydrochloride salt according to any one of claims 54-58, wherein Form A of Compound 9 hydrochloride salt is characterized by an X-ray powder diffractogram having a signal at at least four two-theta values chosen from 6.0±0.2, 7.8±0.2, 15.1±0.2, 15.5±0.2, 17.3±0.2, and 21.5±0.2.

64. The hydrochloride salt according to any one of claims 54-58, wherein Form A of Compound 9 hydrochloride salt is characterized by a DSC substantially similar to that in FIG. 8.

65. The hydrochloride salt according to any one of claims 54-58, wherein Form A of Compound 9 hydrochloride salt is characterized by a DSC having a transitionary melting onset at about 66° C. and/or a peak at about 85° C.

66. The hydrochloride salt according to any one of claims 54-58, wherein Form A of Compound 9 hydrochloride salt is characterized by a DSC having a terminal melting onset at about 130° C. and/or a peak at about 141° C.

67. The hydrochloride salt according to any one of claims 54-58, wherein Form A of Compound 9 hydrochloride salt is characterized by a DSC having a peak in a range of 129° C. to 142° C.

68. The hydrochloride salt according to any one of claims 54-58, wherein Form A of Compound 9 hydrochloride salt is characterized by a TGA substantially similar to that in FIG. 8.

69. The hydrochloride salt according to any one of claims 54-58, wherein Form A of Compound 9 hydrochloride salt is characterized by a TGA showing a weight loss of about 13.5% w/w from ambient temperature up to 155° C.

70. A pharmaceutical composition comprising the hydrochloride salt of Compound 9 according to any one of claims 54 to 69 and a pharmaceutically acceptable carrier.

71. A method of treating a hypoxia-related pathology comprising administering to a subject in need thereof an effective amount of Form A of Compound 9 hydrochloride salt according to any one of claims 54 to 69 or a pharmaceutical composition according to claim 70.

72. A method of modulating HIF activity comprising administering to a subject in need thereof an effective amount of Form A of Compound 9 hydrochloride salt according to any one of claims 54 to 69 or a pharmaceutical composition according to claim 70.

73. A method of treating cancer comprising administering to a subject in need thereof an effective amount of Form A of Compound 9 hydrochloride salt according to any one of claims 54 to 69 or a pharmaceutical composition according to claim 70.

74. The method according to claim 73, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

75. Form B of Compound 9 hydrochloride salt:

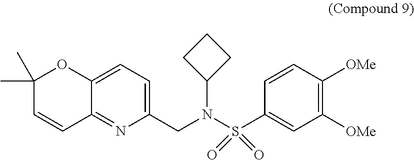 [SMILES: COc1ccc(S(=O)(=O)N(Cc2ccc3c(n2)C=CC(C)(C)O3)C2CCC2)cc1OC]

76. Form B of Compound 9 hydrochloride salt according to claim 75, characterized by an X-ray powder diffractogram substantially similar to that in FIG. 9.

77. Form B of Compound 9 hydrochloride salt according to claim 75, characterized by an X-ray diffractogram having a signal at 8.7±0.2, 9.1±0.2, 10.0±0.2, 15.8±0.2, 17.5±0.2, 18.2±0.2, 20.1±0.2, 20.7±0.2, and 21.3±0.2 two-theta.

78. Form B of Compound 9 hydrochloride salt according to claim 75, characterized by an X-ray powder diffractogram having a signal at at least two two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2.

79. Form B of Compound 9 hydrochloride salt according to claim 75, characterized by an X-ray powder diffractogram having a signal at at least three two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2.

80. Form B of Compound 9 hydrochloride salt according to claim 75, characterized by an X-ray powder diffractogram having a signal at at least four two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2.

81. Form B of Compound 9 hydrochloride salt according to claim 75, characterized by a DSC substantially similar to that in FIG. 10.

82. Form B of Compound 9 hydrochloride salt according to claim 75, characterized by a DSC having a transitionary melting onset at about 160° C. and/or a peak at about 165° C.

83. Form B of Compound 9 hydrochloride salt according to claim 75, characterized by a DSC having a terminal melting onset at about 183° C. and/or a peak at about 186° C.

84. Form B of Compound 9 hydrochloride salt according to claim 75, characterized by a DSC having a peak in a range of 184° C. to 187° C.

85. Form B of Compound 9 hydrochloride salt according to claim 75, characterized by a TGA substantially similar to that in FIG. 10.

86. Form B of Compound 9 hydrochloride salt according to claim 75, characterized by a TGA showing a weight loss of about 8.9% w/w from ambient temperature up to 169° C.

87. A pharmaceutical composition comprising Form B of Compound 9 hydrochloride salt according to any one of claims 75 to 86 and a pharmaceutically acceptable carrier.

88. A method of treating a hypoxia-related pathology comprising administering to a subject in need thereof an effective amount of Form B of Compound 9 hydrochloride salt according to any one of claims 75 to 86 or a pharmaceutical composition according to claim 87.

89. A method of modulating HIF activity comprising administering to a subject in need thereof an effective amount of Form B of Compound 9 hydrochloride salt according to any one of claims 75 to 86 or a pharmaceutical composition according to claim 87.

90. A method of treating cancer comprising administering to a subject in need thereof an effective amount of Form B of Compound 9 hydrochloride salt according to any one of claims 75 to 86 or a pharmaceutical composition according to claim 87.

91. The method according to claim 90, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

92. A hydrochloride salt of compound 9: 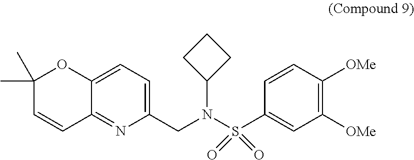 [SMILES: COc1ccc(S(=O)(=O)N(Cc2ccc3c(n2)C=CC(C)(C)O3)C2CCC2)cc1OC]
wherein hydrochloride salt of compound 9 comprises at least Form B.

93. The hydrochloride salt according to claim 92, wherein the hydrochloride salt of compound 9 consists essentially of Form B.

94. The hydrochloride salt according to claim 92, wherein the hydrochloride salt of compound 9 comprises at least 90% Form B.

95. The hydrochloride salt according to claim 92, wherein the hydrochloride salt of compound 9 comprises at least 95% Form B.

96. The hydrochloride salt according to claim 92, wherein the hydrochloride salt of compound 9 comprises at least 98% Form B.

97. The hydrochloride salt according to any one of claims 92-96, wherein Form B of Compound 9 hydrochloride salt is characterized by an X-ray powder diffractogram substantially similar to that in FIG. 9.

98. The hydrochloride salt according to any one of claims 92-96, wherein Form B of Compound 9 hydrochloride salt is characterized by an X-ray diffractogram having a signal at 8.7±0.2, 9.1±0.2, 10.0±0.2, 15.8±0.2, 17.5±0.2, 18.2±0.2, 20.1±0.2, 20.7±0.2, and 21.3±0.2 two-theta.

99. The hydrochloride salt according to any one of claims 92-96, wherein Form B of Compound 9 hydrochloride salt is characterized by an X-ray powder diffractogram having a signal at at least two two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2.

100. The hydrochloride salt according to any one of claims 92-96, wherein Form B of Compound 9 hydrochloride salt is characterized by an X-ray powder diffractogram having a signal at at least three two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2.

101. The hydrochloride salt according to any one of claims 92-96, wherein Form B of Compound 9 hydrochloride salt is characterized by an X-ray powder diffractogram having a signal at at least four two-theta values chosen from 8.7±0.2, 9.1±0.2, 10.0±0.2, 15.8±0.2, 17.5±0.2, 18.2±0.2, 20.1±0.2, 20.7±0.2, and 21.3±0.2.

102. The hydrochloride salt according to any one of claims 92-96, wherein Form B of Compound 9 hydrochloride salt is characterized by a DSC substantially similar to that in FIG. 10.

103. The hydrochloride salt according to any one of claims 92-96, wherein Form B of Compound 9 hydrochloride salt is characterized by a DSC having a transitionary melting onset at about 160° C. and/or a peak at about 165° C.

104. The hydrochloride salt according to any one of claims 92-96, wherein Form B of Compound 9 hydrochloride salt is characterized by a DSC having terminal melting onset at about 183° C. and/or a peak at about 186° C.

105. The hydrochloride salt according to any one of claims 92-96, wherein Form B of Compound 9 hydrochloride salt is characterized by a DSC having peak in a range of 184° C. to 187° C.

106. The hydrochloride salt according to any one of claims 92-96, wherein Form B of Compound 9 hydrochloride salt is characterized by a TGA substantially similar to that in FIG. 10.

107. The hydrochloride salt according to any one of claims 92-96, wherein Form B of Compound 9 hydrochloride salt is characterized by a TGA showing a weight loss of about 8.9% w/w from ambient temperature up to 169° C.

108. A pharmaceutical composition comprising the hydrochloride salt of Compound 9 according to any one of claims 92 to 107 and a pharmaceutically acceptable carrier.

109. A method of treating a hypoxia-related pathology comprising administering to a subject in need thereof an effective amount of Form B of Compound 9 hydrochloride salt according to any one of claims 92 to 107 or a pharmaceutical composition according to claim 108.

110. A method of modulating HIF activity comprising administering to a subject in need thereof an effective amount of Form B of Compound 9 hydrochloride salt according to any one of claims 92 to 107 or a pharmaceutical composition according to claim 108.

111. A method of treating cancer comprising administering to a subject in need thereof an effective amount of Form B of Compound 9 hydrochloride salt according to any one of claims 92 to 107 or a pharmaceutical composition according to claim 108.

112. The method according to claim 111, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

113. Form C of Compound 9 hydrochloride salt:

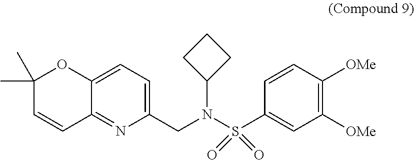 [SMILES: COc1ccc(S(=O)(=O)N(Cc2ccc3c(n2)C=CC(C)(C)O3)C2CCC2)cc1OC]

114. Form C of Compound 9 hydrochloride salt according to claim 113, characterized by an X-ray powder diffractogram substantially similar to that in FIG. 11.

115. Form C of Compound 9 hydrochloride salt according to claim 113, characterized by an X-ray diffractogram having a signal at 10.2±0.2, 11.6±0.2, 12.0±0.2, 13.7±0.2, 16.6±0.2, 17.1±0.2, and 23.3±0.2 two-theta.

116. Form C of Compound 9 hydrochloride salt according to claim 113, characterized by an X-ray powder diffractogram having a signal at at least two two-theta values chosen from 10.2±0.2, 11.6±0.2, 12.0±0.2, 13.7±0.2, 16.6±0.2, 17.1±0.2, and 23.3±0.2.

117. Form C of Compound 9 hydrochloride salt according to claim 113, characterized by an X-ray powder diffractogram having a signal at at least three two-theta values chosen from 10.2±0.2, 11.6±0.2, 12.0±0.2, 13.7±0.2, 16.6±0.2, 17.1±0.2, and 23.3±0.2.

118. Form C of Compound 9 hydrochloride salt according to claim 113, characterized by an X-ray powder diffractogram having a signal at at least four two-theta values chosen from 10.2±0.2, 11.6±0.2, 12.0±0.2, 13.7±0.2, 16.6±0.2, 17.1±0.2, and 23.3±0.2.

119. Form C of Compound 9 hydrochloride salt according to claim 113, characterized by a DSC substantially similar to that in FIG. 12.

120. Form C of Compound 9 hydrochloride salt according to claim 113, characterized by a DSC having a transitionary melting onset at about 132° C. and/or a peak at about 137° C.

121. Form C of Compound 9 hydrochloride salt according to claim 113, characterized by a DSC having a terminal melting onset at about 181° C. and/or a peak at about 188° C.

122. Form C of Compound 9 hydrochloride salt according to claim 113, characterized by a DSC having a peak in a range of 180° C. to 189° C.

123. Form C of Compound 9 hydrochloride salt according to claim 113, characterized by a TGA substantially similar to that in FIG. 12.

124. Form C of Compound 9 hydrochloride salt according to claim 113, characterized by a TGA showing a weight loss of about 7.8% w/w from ambient temperature up to 181° C.

125. A pharmaceutical composition comprising Form C of Compound 9 hydrochloride salt according to any one of claims 113 to 124 and a pharmaceutically acceptable carrier.

126. A method of treating a hypoxia-related pathology comprising administering to a subject in need thereof an effective amount of Form C of Compound 9 hydrochloride salt according to any one of claims 113 to 124 or a pharmaceutical composition according to claim 125.

127. A method of modulating HIF activity comprising administering to a subject in need thereof an effective amount of Form C of Compound 9 hydrochloride salt according to any one of claims 113 to 124 or a pharmaceutical composition according to claim 125.

128. A method of treating cancer comprising administering to a subject in need thereof an effective amount of Form C of Compound 9 hydrochloride salt according to any one of claims 113 to 124 or a pharmaceutical composition according to claim 125.

129. The method according to claim 128, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

130. A hydrochloride salt of compound 9: 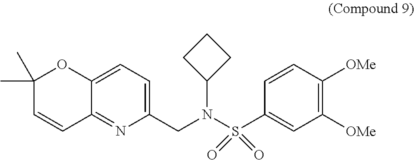 [SMILES: COc1ccc(S(=O)(=O)N(Cc2ccc3c(n2)C=CC(C)(C)O3)C2CCC2)cc1OC]
wherein hydrochloride salt of compound 9 comprises at least Form C.

131. The hydrochloride salt according to claim 130, wherein the hydrochloride salt of compound 9 consists essentially of Form C.

132. The hydrochloride salt according to claim 130, wherein the hydrochloride salt of compound 9 comprises at least 90% Form C.

133. The hydrochloride salt according to claim 130, wherein the hydrochloride salt of compound 9 comprises at least 95% Form C.

134. The hydrochloride salt according to claim 130, wherein the hydrochloride salt of compound 9 comprises at least 98% Form C.

135. The hydrochloride salt according to any one of claims 130-134, wherein Form C of Compound 9 hydrochloride salt is characterized by an X-ray powder diffractogram substantially similar to that in FIG. 11.

136. The hydrochloride salt according to any one of claims 130-134, wherein Form C of Compound 9 hydrochloride salt is characterized by an X-ray diffractogram having a signal at 10.2±0.2, 11.6±0.2, 12.0±0.2, 13.7±0.2, 16.6±0.2, 17.1±0.2, and 23.3±0.2 two-theta.

137. The hydrochloride salt according to any one of claims 130-134, wherein Form C of Compound 9 hydrochloride salt is characterized by an X-ray powder diffractogram having a signal at at least two two-theta values chosen from 10.2±0.2, 11.6±0.2, 12.0±0.2, 13.7±0.2, 16.6±0.2, 17.1±0.2, and 23.3±0.2.

138. The hydrochloride salt according to any one of claims 130-134, wherein Form C of Compound 9 hydrochloride salt is characterized by an X-ray powder diffractogram having a signal at at least three two-theta values chosen from 10.2±0.2, 11.6±0.2, 12.0±0.2, 13.7±0.2, 16.6±0.2, 17.1±0.2, and 23.3±0.2.

139. The hydrochloride salt according to any one of claims 130-134, wherein Form C of Compound 9 hydrochloride salt is characterized by an X-ray powder diffractogram having a signal at at least four two-theta values chosen from 10.2±0.2, 11.6±0.2, 12.0±0.2, 13.7±0.2, 16.6±0.2, 17.1±0.2, and 23.3±0.2.

140. The hydrochloride salt according to any one of claims 130-134, wherein Form C of Compound 9 hydrochloride salt is characterized by a DSC substantially similar to that in FIG. 12.

141. The hydrochloride salt according to any one of claims 130-134, wherein Form C of Compound 9 hydrochloride salt is characterized by a DSC having a transitionary melting onset at about 132° C. and/or a peak at about 137° C.

142. The hydrochloride salt according to any one of claims 130-134, wherein Form C of Compound 9 hydrochloride salt is characterized by a DSC having terminal melting onset at about 181° C. and/or a peak at about 188° C.

143. The hydrochloride salt according to any one of claims 130-134, wherein Form C of Compound 9 hydrochloride salt is characterized by a DSC having peak in a range of 180° C. to 189° C.

144. The hydrochloride salt according to any one of claims 130-134, wherein Form C of Compound 9 hydrochloride salt is characterized by a TGA substantially similar to that in FIG. 12.

145. The hydrochloride salt according to any one of claims 130-134, wherein Form C of Compound 9 hydrochloride salt is characterized by a TGA showing a weight loss of about 7.8% w/w from ambient temperature up to 181° C.

146. A pharmaceutical composition comprising the hydrochloride salt of Compound 9 according to any one of claims 130 to 145 and a pharmaceutically acceptable carrier.

147. A method of treating a hypoxia-related pathology comprising administering to a subject in need thereof an effective amount of Form C of Compound 9 hydrochloride salt according to any one of claims 130 to 145 or a pharmaceutical composition according to claim 146.

148. A method of modulating HIF activity comprising administering to a subject in need thereof an effective amount of Form C of Compound 9 hydrochloride salt according to any one of claims 130 to 145 or a pharmaceutical composition according to claim 146.

149. A method of treating cancer comprising administering to a subject in need thereof an effective amount of Form C of Compound 9 hydrochloride salt according to any one of claims 130 to 145 or a pharmaceutical composition according to claim 146.

150. The method according to claim 149, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

151. Form A of Compound 9 sulfate salt:

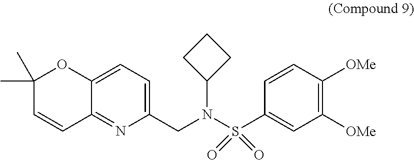 [SMILES: COc1ccc(S(=O)(=O)N(Cc2ccc3c(n2)C=CC(C)(C)O3)C2CCC2)cc1OC]

152. Form A of Compound 9 sulfate salt according to claim 151, characterized by an X-ray powder diffractogram substantially similar to that in FIG. 13.

153. Form A of Compound 9 sulfate salt according to claim 151, characterized by an X-ray diffractogram having a signal at 5.7±0.2, 8.6±0.2, 8.8±0.2, 10.3±0.2, 15.3±0.2, 16.4±0.2, 17.1±0.2, and 18.2±0.2 two-theta.

154. Form A of Compound 9 sulfate salt according to claim 151, characterized by an X-ray powder diffractogram having a signal at at least two two-theta values chosen from 5.7±0.2, 8.6±0.2, 8.8±0.2, 10.3±0.2, 15.3±0.2, 16.4±0.2, 17.1±0.2, and 18.2±0.2.

155. Form A of Compound 9 sulfate salt according to claim 151, characterized by an X-ray powder diffractogram having a signal at at least three two-theta values chosen from 5.7±0.2, 8.6±0.2, 8.8±0.2, 10.3±0.2, 15.3±0.2, 16.4±0.2, 17.1±0.2, and 18.2±0.2.

156. Form A of Compound 9 sulfate salt according to claim 151, characterized by an X-ray powder diffractogram having a signal at at least four two-theta values chosen from 5.7±0.2, 8.6±0.2, 8.8±0.2, 10.3±0.2, 15.3±0.2, 16.4±0.2, 17.1±0.2, and 18.2±0.2.

157. Form A of Compound 9 sulfate salt according to claim 151, characterized by a DSC substantially similar to that in FIG. 14.

158. Form A of Compound 9 sulfate salt according to claim 151, characterized by a DSC having a terminal melting onset at about 177° C. and/or a peak at about 194° C.

159. Form A of Compound 9 sulfate salt according to claim 151, characterized by a DSC having a peak in a range of 170° C. to 195° C.

160. Form A of Compound 9 sulfate salt according to claim 151, characterized by a TGA substantially similar to that in FIG. 14.

161. Form A of Compound 9 sulfate salt according to claim 151, characterized by a TGA showing a weight loss of about 9.9% w/w from ambient temperature up to 130° C.

162. A pharmaceutical composition comprising Form A of Compound 9 sulfate salt according to any one of claims 151 to 161 and a pharmaceutically acceptable carrier.

163. A method of treating a hypoxia-related pathology comprising administering to a subject in need thereof an effective amount of Form A of Compound 9 sulfate salt according to any one of claims 151 to 161 or a pharmaceutical composition according to claim 162.

164. A method of modulating HIF activity comprising administering to a subject in need thereof an effective amount of Form A of Compound 9 sulfate salt according to any one of claims 151 to 161 or a pharmaceutical composition according to claim 162.

165. A method of treating cancer comprising administering to a subject in need thereof an effective amount of Form A of Compound 9 sulfate salt according to any one of claims 151 to 161 or a pharmaceutical composition according to claim 162.

166. The method according to claim 165, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

167. A sulfate salt of compound 9: 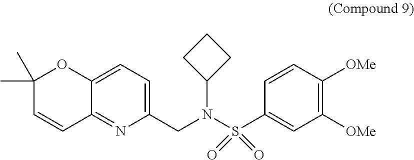 [SMILES: COc1ccc(S(=O)(=O)N(Cc2ccc3c(n2)C=CC(C)(C)O3)C2CCC2)cc1OC]
wherein sulfate salt of compound 9 comprises at least Form A.

168. The sulfate salt according to claim 167, wherein the sulfate salt of compound 9 consists essentially of Form A.

169. The sulfate salt according to claim 167, wherein the sulfate salt of compound 9 comprises at least 90% Form A.

170. The sulfate salt according to claim 167, wherein the sulfate salt of compound 9 comprises at least 95% Form A.

171. The sulfate salt according to claim 167, wherein the sulfate salt of compound 9 comprises at least 98% Form A.

172. The sulfate salt according to any one of claims 167-171, wherein Form A of Compound 9 sulfate salt is characterized by an X-ray powder diffractogram substantially similar to that in FIG. 13.

173. The sulfate salt according to any one of claims 167-171, wherein Form A of Compound 9 sulfate salt is characterized by an X-ray diffractogram having a signal at 5.7±0.2, 8.6±0.2, 8.8±0.2, 10.3±0.2, 15.3±0.2, 16.4±0.2, 17.1±0.2, and 18.2±0.2 two-theta.

174. The sulfate salt according to any one of claims 167-171, wherein Form A of Compound 9 sulfate salt is characterized by an X-ray powder diffractogram having a signal at at least two two-theta values chosen from 5.7±0.2, 8.6±0.2, 8.8±0.2, 10.3±0.2, 15.3±0.2, 16.4±0.2, 17.1±0.2, and 18.2±0.2.

175. The sulfate salt according to any one of claims 167-171, wherein Form A of Compound 9 sulfate salt is characterized by an X-ray powder diffractogram having a signal at at least three two-theta values chosen from 5.7±0.2, 8.6±0.2, 8.8±0.2, 10.3±0.2, 15.3±0.2, 16.4±0.2, 17.1±0.2, and 18.2±0.2.

176. The sulfate salt according to any one of claims 167-171, wherein Form A of Compound 9 sulfate salt is characterized by an X-ray powder diffractogram having a signal at at least four two-theta values chosen from 5.7±0.2, 8.6±0.2, 8.8±0.2, 10.3±0.2, 15.3±0.2, 16.4±0.2, 17.1±0.2, and 18.2±0.2.

177. The sulfate salt according to any one of claims 167-171, wherein Form A of Compound 9 sulfate salt is characterized by a DSC substantially similar to that in FIG. 14.

178. The sulfate salt according to any one of claims 167-171, wherein Form A of Compound 9 sulfate salt is characterized by a DSC having terminal melting onset at about 177° C. and/or a peak at about 194° C.

179. The sulfate salt according to any one of claims 167-171, wherein Form A of Compound 9 sulfate salt is characterized by a DSC having peak in a range of 170° C. to 195° C.

180. The sulfate salt according to any one of claims 167-171, wherein Form A of Compound 9 sulfate salt is characterized by a TGA substantially similar to that in FIG. 14.

181. The sulfate salt according to any one of claims 167-171, wherein Form A of Compound 9 sulfate salt is characterized by a TGA showing a weight loss of about 9.9% w/w from ambient temperature up to 130° C.

182. A pharmaceutical composition comprising the sulfate salt of Compound 9 according to any one of claims 167 to 181 and a pharmaceutically acceptable carrier.

183. A method of treating a hypoxia-related pathology comprising administering to a subject in need thereof an effective amount of Form A of Compound 9 sulfate salt according to any one of claims 167 to 181 or a pharmaceutical composition according to claim 182.

184. A method of modulating HIF activity comprising administering to a subject in need thereof an effective amount of Form A of Compound 9 sulfate salt according to any one of claims 167 to 181 or a pharmaceutical composition according to claim 182.

185. A method of treating cancer comprising administering to a subject in need thereof an effective amount of Form A of Compound 9 sulfate salt according to any one of claims 167 to 181 or a pharmaceutical composition according to claim 182.

186. The method according to claim 185, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

187. Form B of Compound 9 sulfate salt:

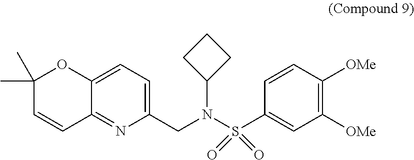 [SMILES: COc1ccc(S(=O)(=O)N(Cc2ccc3c(n2)C=CC(C)(C)O3)C2CCC2)cc1OC]

188. Form B of Compound 9 sulfate salt according to claim 187, characterized by an X-ray powder diffractogram substantially similar to that in FIG. 16.

189. Form B of Compound 9 sulfate salt according to claim 187, characterized by an X-ray diffractogram having a signal at 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2 two-theta.

190. Form B of Compound 9 sulfate salt according to claim 187, characterized by an X-ray powder diffractogram having a signal at at least two two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2.

191. Form B of Compound 9 sulfate salt according to claim 187, characterized by an X-ray powder diffractogram having a signal at at least three two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2.

192. Form B of Compound 9 sulfate salt according to claim 187, characterized by an X-ray powder diffractogram having a signal at at least four two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2.

193. A pharmaceutical composition comprising Form B of Compound 9 sulfate salt according to any one of claims 187 to 192 and a pharmaceutically acceptable carrier.

194. A method of treating a hypoxia-related pathology comprising administering to a subject in need thereof an effective amount of Form B of Compound 9 sulfate salt according to any one of claims 187 to 192 or a pharmaceutical composition according to claim 193.

195. A method of modulating HIF activity comprising administering to a subject in need thereof an effective amount of Form B of Compound 9 sulfate salt according to any one of claims 187 to 192 or a pharmaceutical composition according to claim 193.

196. A method of treating cancer comprising administering to a subject in need thereof an effective amount of Form B of Compound 9 sulfate salt according to any one of claims 187 to 192 or a pharmaceutical composition according to claim 193.

197. The method according to claim 196, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

198. A sulfate salt of compound 9: 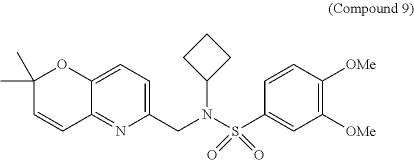 [SMILES: COc1ccc(S(=O)(=O)N(Cc2ccc3c(n2)C=CC(C)(C)O3)C2CCC2)cc1OC]
wherein sulfate salt of compound 9 comprises at least Form B.

199. The sulfate salt according to claim 198, wherein the sulfate salt of compound 9 consists essentially of Form B.

200. The sulfate salt according to claim 198, wherein the sulfate salt of compound 9 comprises at least 90% Form B.

201. The sulfate salt according to claim 198, wherein the sulfate salt of compound 9 comprises at least 95% Form B.

202. The sulfate salt according to claim 198, wherein the sulfate salt of compound 9 comprises at least 98% Form B.

203. The sulfate salt according to any one of claims 198-202, wherein Form B of Compound 9 sulfate salt is characterized by an X-ray powder diffractogram substantially similar to that in FIG. 16.

204. The sulfate salt according to any one of claims 198-202, wherein Form B of Compound 9 sulfate salt is characterized by an X-ray diffractogram having a signal at 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2 two-theta.

205. The sulfate salt according to any one of claims 198-202, wherein Form B of Compound 9 sulfate salt is characterized by an X-ray powder diffractogram having a signal at at least two two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2.

206. The sulfate salt according to any one of claims 198-202, wherein Form B of Compound 9 sulfate salt is characterized by an X-ray powder diffractogram having a signal at at least three two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2.

207. The sulfate salt according to any one of claims 198-202, wherein Form B of Compound 9 sulfate salt is characterized by an X-ray powder diffractogram having a signal at at least four two-theta values chosen from 7.4±0.2, 9.5±0.2, 11.4±0.2, 12.4±0.2, 12.7±0.2, 15.5±0.2, 16.2±0.2, 17.3±0.2, 18.0±0.2, 19.1±0.2, 22.5±0.2, 23.8±0.2, and 26.2±0.2.

208. A pharmaceutical composition comprising the sulfate salt of Compound 9 according to any one of claims 198 to 207 and a pharmaceutically acceptable carrier.

209. A method of treating a hypoxia-related pathology comprising administering to a subject in need thereof an effective amount of Form B of Compound 9 sulfate salt according to any one of claims 198 to 207 or a pharmaceutical composition according to claim 208.

210. A method of modulating HIF activity comprising administering to a subject in need thereof an effective amount of Form B of Compound 9 sulfate salt according to any one of claims 198 to 207 or a pharmaceutical composition according to claim 208.

211. A method of treating cancer comprising administering to a subject in need thereof an effective amount of Form B of Compound 9 sulfate salt according to any one of claims 198 to 207 or a pharmaceutical composition according to claim 208.

212. The method according to claim 211, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

213. Form A of Compound 9 tosylate salt:

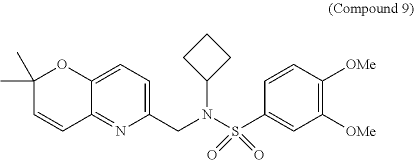 [SMILES: COc1ccc(S(=O)(=O)N(Cc2ccc3c(n2)C=CC(C)(C)O3)C2CCC2)cc1OC]

214. Form A of Compound 9 tosylate salt according to claim 213, characterized by an X-ray powder diffractogram substantially similar to that in FIG. 17.

215. Form A of Compound 9 tosylate salt according to claim 213, characterized by an X-ray diffractogram having a signal at 6.4±0.2, 7.3±0.2, 9.3±0.2, 17.9±0.2, 19.5±0.2, 21.0±0.2, 21.4±0.2, and 27.3±0.2 two-theta.

216. Form A of Compound 9 tosylate salt according to claim 213, characterized by an X-ray powder diffractogram having a signal at at least two two-theta values chosen from 6.4±0.2, 7.3±0.2, 9.3±0.2, 17.9±0.2, 19.5±0.2, 21.0±0.2, 21.4±0.2, and 27.3±0.2.

217. Form A of Compound 9 tosylate salt according to claim 213, characterized by an X-ray powder diffractogram having a signal at at least three two-theta values chosen from 6.4±0.2, 7.3±0.2, 9.3±0.2, 17.9±0.2, 19.5±0.2, 21.0±0.2, 21.4±0.2, and 27.3±0.2.

218. Form A of Compound 9 tosylate salt according to claim 213, characterized by an X-ray powder diffractogram having a signal at at least four two-theta values chosen from 6.4±0.2, 7.3±0.2, 9.3±0.2, 17.9±0.2, 19.5±0.2, 21.0±0.2, 21.4±0.2, and 27.3±0.2.

219. Form A of Compound 9 tosylate salt according to claim 213, characterized by a DSC substantially similar to that in FIG. 18.

220. Form A of Compound 9 tosylate salt according to claim 213, characterized by a DSC having a melting onset at about 76° C. and/or a peak at about 91° C.

221. Form A of Compound 9 tosylate salt according to claim 213, characterized by a DSC having a peak in a range of 75° C. to 92° C.

222. Form A of Compound 9 tosylate salt according to claim 213, characterized by a TGA substantially similar to that in FIG. 18.

223. Form A of Compound 9 tosylate salt according to claim 213, characterized by a TGA showing a weight loss of about 8.3% w/w from ambient temperature up to 154° C.

224. A pharmaceutical composition comprising Form A of Compound 9 tosylate salt according to any one of claims 213 to 223 and a pharmaceutically acceptable carrier.

225. A method of treating a hypoxia-related pathology comprising administering to a subject in need thereof an effective amount of Form A of Compound 9 tosylate salt according to any one of claims 213 to 223 or a pharmaceutical composition according to claim 224.

226. A method of modulating HIF activity comprising administering to a subject in need thereof an effective amount of Form A of Compound 9 tosylate salt according to any one of claims 213 to 223 or a pharmaceutical composition according to claim 224.

227. A method of treating cancer comprising administering to a subject in need thereof an effective amount of Form A of Compound 9 tosylate salt according to any one of claims 213 to 223 or a pharmaceutical composition according to claim 224.

228. The method according to claim 227, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

229. A tosylate salt of compound 9: 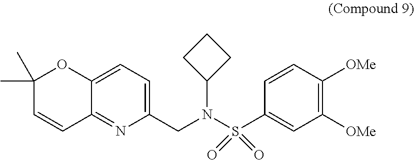 [SMILES: COc1ccc(S(=O)(=O)N(Cc2ccc3c(n2)C=CC(C)(C)O3)C2CCC2)cc1OC]
wherein tosylate salt of compound 9 comprises at least Form A.

230. The tosylate salt according to claim 229, wherein the tosylate salt of compound 9 consists essentially of Form A.

231. The tosylate salt according to claim 229, wherein the tosylate salt of compound 9 comprises at least 90% Form A.

232. The tosylate salt according to claim 229, wherein the tosylate salt of compound 9 comprises at least 95% Form A.

233. The tosylate salt according to claim 229, wherein the tosylate salt of compound 9 comprises at least 98% Form A.

234. The tosylate salt according to any one of claims 229-233, wherein Form A of Compound 9 tosylate salt is characterized by an X-ray powder diffractogram substantially similar to that in FIG. 17.

235. The tosylate salt according to any one of claims 229-233, wherein Form A of Compound 9 tosylate salt is characterized by an X-ray diffractogram having a signal at 6.4±0.2, 7.3±0.2, 9.3±0.2, 17.9±0.2, 19.5±0.2, 21.0±0.2, 21.4±0.2, and 27.3±0.2 two-theta.

236. The tosylate salt according to any one of claims 229-233, wherein Form A of Compound 9 tosylate salt is characterized by an X-ray powder diffractogram having a signal at at least two two-theta values chosen from 6.4±0.2, 7.3±0.2, 9.3±0.2, 17.9±0.2, 19.5±0.2, 21.0±0.2, 21.4±0.2, and 27.3±0.2.

237. The tosylate salt according to any one of claims 229-233, wherein Form A of Compound 9 tosylate salt is characterized by an X-ray powder diffractogram having a signal at at least three two-theta values chosen from 6.4±0.2, 7.3±0.2, 9.3±0.2, 17.9±0.2, 19.5±0.2, 21.0±0.2, 21.4±0.2, and 27.3±0.2.

238. The tosylate salt according to any one of claims 229-233, wherein Form A of Compound 9 tosylate salt is characterized by an X-ray powder diffractogram having a signal at at least four two-theta values chosen from 6.4±0.2, 7.3±0.2, 9.3±0.2, 17.9±0.2, 19.5±0.2, 21.0±0.2, 21.4±0.2, and 27.3±0.2.

239. The tosylate salt according to any one of claims 229-233, wherein Form A of Compound 9 tosylate salt is characterized by a DSC substantially similar to that in FIG. 18.

240. The tosylate salt according to any one of claims 229-233, wherein Form A of Compound 9 tosylate salt is characterized by a DSC having melting onset at about 76° C. and/or a peak at about 91° C.

241. The tosylate salt according to any one of claims 229-233, wherein Form A of Compound 9 tosylate salt is characterized by a DSC having peak in a range of 75° C. to 92° C.

242. The tosylate salt according to any one of claims 229-233, wherein Form A of Compound 9 tosylate salt is characterized by a TGA substantially similar to that in FIG. 18.

243. The tosylate salt according to any one of claims 229-233, wherein Form A of Compound 9 tosylate salt is characterized by a TGA showing a weight loss of about 8.3% w/w from ambient temperature up to 154° C.

244. A pharmaceutical composition comprising the tosylate salt of Compound 9 according to any one of claims 229 to 243 and a pharmaceutically acceptable carrier.

245. A method of treating a hypoxia-related pathology comprising administering to a subject in need thereof an effective amount of Form A of Compound 9 tosylate salt according to any one of claims 229 to 243 or a pharmaceutical composition according to claim 244.

246. A method of modulating HIF activity comprising administering to a subject in need thereof an effective amount of Form A of Compound 9 tosylate salt according to any one of claims 229 to 243 or a pharmaceutical composition according to claim 244.

247. A method of treating cancer comprising administering to a subject in need thereof an effective amount of Form A of Compound 9 tosylate salt according to any one of claims 229 to 243 or a pharmaceutical composition according to claim 244.

248. The method according to claim 247, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

249. Form A of Compound 9 naphthalene-1,5-disulfonic acid salt:

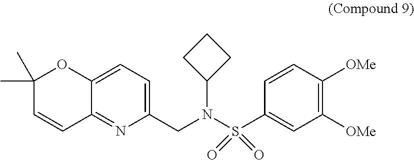 [SMILES: COc1ccc(S(=O)(=O)N(Cc2ccc3c(n2)C=CC(C)(C)O3)C2CCC2)cc1OC]

250. Form A of Compound 9 naphthalene-1,5-disulfonic acid salt according to claim 249, characterized by an X-ray powder diffractogram substantially similar to that in FIG. 20.

251. Form A of Compound 9 naphthalene-1,5-disulfonic acid salt according to claim 249, characterized by an X-ray diffractogram having a signal at 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2 two-theta.

252. Form A of Compound 9 naphthalene-1,5-disulfonic acid salt according to claim 249, characterized by an X-ray powder diffractogram having a signal at at least two two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2.

253. Form A of Compound 9 naphthalene-1,5-disulfonic acid salt according to claim 249, characterized by an X-ray powder diffractogram having a signal at at least three two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2.

254. Form A of Compound 9 naphthalene-1,5-disulfonic acid salt according to claim 249, characterized by an X-ray powder diffractogram having a signal at at least four two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2.

255. Form A of Compound 9 naphthalene-1,5-disulfonic acid salt according to claim 249, characterized by a DSC substantially similar to that in FIG. 21.

256. Form A of Compound 9 naphthalene-1,5-disulfonic acid salt according to claim 249, characterized by a DSC having a melting onset at about 136° C. and/or a peak at about 140° C.

257. Form A of Compound 9 naphthalene-1,5-disulfonic acid salt according to claim 249, characterized by a DSC having a peak in a range of 135° C. to 141° C.

258. Form A of Compound 9 naphthalene-1,5-disulfonic acid salt according to claim 249, characterized by a TGA substantially similar to that in FIG. 21.

259. Form A of Compound 9 naphthalene-1,5-disulfonic acid salt according to claim 249, characterized by a TGA showing a weight loss of about 6.5% w/w from ambient temperature up to 154° C.

260. A pharmaceutical composition comprising Form A of Compound 9 naphthalene-1,5-disulfonic acid salt according to any one of claims 249 to 259 and a pharmaceutically acceptable carrier.

261. A method of treating a hypoxia-related pathology comprising administering to a subject in need thereof an effective amount of Form A of Compound 9 naphthalene-1,5-disulfonic acid salt according to any one of claims 249 to 259 or a pharmaceutical composition according to claim 260.

262. A method of modulating HIF activity comprising administering to a subject in need thereof an effective amount of Form A of Compound 9 naphthalene-1,5-disulfonic acid salt according to any one of claims 249 to 259 or a pharmaceutical composition according to claim 260.

263. A method of treating cancer comprising administering to a subject in need thereof an effective amount of Form A of Compound 9 naphthalene-1,5-disulfonic acid salt according to any one of claims 249 to 259 or a pharmaceutical composition according to claim 260.

264. The method according to claim 263, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.

265. A naphthalene-1,5-disulfonic acid salt of compound 9: 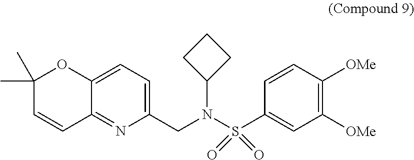 [SMILES: COc1ccc(S(=O)(=O)N(Cc2ccc3c(n2)C=CC(C)(C)O3)C2CCC2)cc1OC]
wherein naphthalene-1,5-disulfonic acid salt of compound 9 comprises at least Form A.

266. The naphthalene-1,5-disulfonic acid salt according to claim 265, wherein the naphthalene-1,5-disulfonic acid salt of compound 9 consists essentially of Form A.

267. The naphthalene-1,5-disulfonic acid salt according to claim 265, wherein the naphthalene-1,5-disulfonic acid salt of compound 9 comprises at least 90% Form A.

268. The naphthalene-1,5-disulfonic acid salt according to claim 265, wherein the naphthalene-1,5-disulfonic acid salt of compound 9 comprises at least 95% Form A.

269. The naphthalene-1,5-disulfonic acid salt according to claim 265, wherein the naphthalene-1,5-disulfonic acid salt of compound 9 comprises at least 98% Form A.

270. The naphthalene-1,5-disulfonic acid salt according to any one of claims 265-269, wherein Form A of Compound 9 naphthalene-1,5-disulfonic acid salt is characterized by an X-ray powder diffractogram substantially similar to that in FIG. 20.

271. The naphthalene-1,5-disulfonic acid salt according to any one of claims 265-269, wherein Form A of Compound 9 naphthalene-1,5-disulfonic acid salt is characterized by an X-ray diffractogram having a signal at 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2 two-theta.

272. The naphthalene-1,5-disulfonic acid salt according to any one of claims 265-269, wherein Form A of Compound 9 naphthalene-1,5-disulfonic acid salt is characterized by an X-ray powder diffractogram having a signal at at least two two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2.

273. The naphthalene-1,5-disulfonic acid salt according to any one of claims 265-269, wherein Form A of Compound 9 naphthalene-1,5-disulfonic acid salt is characterized by an X-ray powder diffractogram having a signal at at least three two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2.

274. The naphthalene-1,5-disulfonic acid salt according to any one of claims 265-269, wherein Form A of Compound 9 naphthalene-1,5-disulfonic acid salt is characterized by an X-ray powder diffractogram having a signal at at least four two-theta values chosen from 9.9±0.2, 10.5±0.2, 12.4±0.2, 13.1±0.2, 15.1±0.2, 15.8±0.2, 17.5±0.2, 17.8±0.2, 19.0±0.2, 19.7±0.2, 20.3±0.2, 21.0±0.2, 22.2±0.2, and 28.2±0.2.

275. The naphthalene-1,5-disulfonic acid salt according to any one of claims 265-269, wherein Form A of Compound 9 naphthalene-1,5-disulfonic acid salt is characterized by a DSC substantially similar to that in FIG. 21.

276. The naphthalene-1,5-disulfonic acid salt according to any one of claims 265-269, wherein Form A of Compound 9 naphthalene-1,5-disulfonic acid salt is characterized by a DSC having melting onset at about 136° C. and/or a peak at about 140° C.

277. The naphthalene-1,5-disulfonic acid salt according to any one of claims 265-269, wherein Form A of Compound 9 naphthalene-1,5-disulfonic acid salt is characterized by a DSC having peak in a range of 135° C. to 141° C.

278. The naphthalene-1,5-disulfonic acid salt according to any one of claims 265-269, wherein Form A of Compound 9 naphthalene-1,5-disulfonic acid salt is characterized by a TGA substantially similar to that in FIG. 21.

279. The naphthalene-1,5-disulfonic acid salt according to any one of claims 265-269, wherein Form A of Compound 9 naphthalene-1,5-disulfonic acid salt is characterized by a TGA showing a weight loss of about 6.5% w/w from ambient temperature up to 154° C.

280. A pharmaceutical composition comprising the naphthalene-1,5-disulfonic acid salt of Compound 9 according to any one of claims 265 to 279 and a pharmaceutically acceptable carrier.

281. A method of treating a hypoxia-related pathology comprising administering to a subject in need thereof an effective amount of Form A of Compound 9 naphthalene-1,5-disulfonic acid salt according to any one of claims 265 to 279 or a pharmaceutical composition according to claim 280.

282. A method of modulating HIF activity comprising administering to a subject in need thereof an effective amount of Form A of Compound 9 naphthalene-1,5-disulfonic acid salt according to any one of claims 265 to 279 or a pharmaceutical composition according to claim 280.

283. A method of treating cancer comprising administering to a subject in need thereof an effective amount of Form A of Compound 9 naphthalene-1,5-disulfonic acid salt according to any one of claims 265 to 279 or a pharmaceutical composition according to claim 280.

284. The method according to claim 283, where the cancer is chosen from bladder cancer, brain cancer, breast cancer, colorectal cancer, cervical cancer, gastrointestinal cancer, genitourinary cancer, head and neck cancer, lung cancer, melanoma, ovarian cancer, pancreatic cancer, prostate cancer, renal cancer, skin cancer, and testicular cancer.
